(19) United States
(12) Patent Application Publication
(10) Pub. No.: US 2024/0327361 A1
(43) Pub. Date: Oct. 3, 2024

(54) RADIOLABELED COMPOSITIONS AND METHODS OF USE THEREOF

(71) Applicant: The Research Foundation for the State University of New York, Albany, NY (US)

(72) Inventor: Nashaat Turkman, Stony Brook, NY (US)

(73) Assignee: The Research Foundation for the State University of New York, Albany, NY (US)

(21) Appl. No.: 18/546,322

(22) PCT Filed: Feb. 15, 2022
(86) PCT No.: PCT/US2022/016464

§ 371 Date: Aug. 14, 2023

Related U.S. Application Data
(60) Provisional application No. 63/149,505, filed on Feb. 15, 2021.

(51) Int. Cl. C07D 271/06; A61K 31/4025; A61K 31/4245; A61K 31/4439; C07D 413/04; C07D 413/12

(52) U.S. Cl. CPC C07D 271/06; A61K 31/4025; A61K 31/4245; A61K 31/4439; C07D 413/04; C07D 413/12

(57) ABSTRACT

The present invention provides a compound which is a class-lla histone deacetylases (HDAC) inhibitor, a process of making the compound, a composition containing the compound and radiolabeling the compound for use in positron emission tomography (PET) imaging, said compound having the structure:

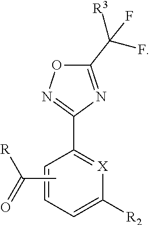

This application claims priority of U.S. Provisional Application No. 63/149,505, filed Feb. 15, 2021, the contents of which are hereby incorporated by reference.

### GOVERNMENT SUPPORT

This invention was made with government support under AG067417-01 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

The dysregulation of class-lla histone deacetylases (HDACs) is implicated in various cancers and in the neurodegenerative diseases of the central nervous system (CNS). Therefore, molecular imaging of class-lla HDACs with positron emission tomography (PET) enable early diagnosis and select patients for therapeutic intervention with targeted class-lla HDAC inhibitors to improve overall outcomes. Recently, 5-trifluoromethyl-1,2,4-oxadiazole (TFMO) containing small molecules were reported to exhibit potent and specific class-lla HDACs inhibition. Herein, TFMO moiety was successfully radiolabeled with F-18 and validated the utility of the new radiochemical design by the radiosynthesis of ¹⁸F-NT57, a radioactive homolog of TMP195, a known TFMO containing potent inhibitor of class-lla HDACs. PET imaging was performed in vivo with the new probe in rats thus mapping the biodistribution of class-lla HDACs in healthy tissues in the periphery and the CNS.

The Human histone deacetylases (HDACs) form a large family of 18 members classified into four classes (I to IV) (Aramsangtienchai, P., et al. 2006; Younes, A., et al. 2016). The class-lla HDACs is a sub family comprised of four enzymes: HDAC-4, HDAC-5, HDAC-7 and HDAC-9 (Li and Yang 2016; Kikuchi, S., et al. 2015; Choi, S. Y., et al. 2018). There is mounting evidence on a key role for the class-Ila HDACs in various cancers and in the disorders of the central nervous system (CNS) including memory and cognitive impairment, dementia and behavioral changes among others (Linares, A., et al. 2019).

Class-lla HDAC-4 and 5 are expressed throughout the brain silencing or truncation led to defect in spatial learning and memory, cognitive impairment and dementia (Bolger T A Y T 2005; Gräff J T L 2013; Sando R G N. 2012). However, despite intense studies, relatively little is known regarding the expression level and distribution of class-lla HDACs in the human brain in its entirety. Although postmortem studies established a link between expression of class-lla HDAC and neurodegenerative diseases such as Alzheimer's and Huntington's (Anderson 2015; Yeh H H Y D, et al. 2013), these studies are limited to one or very few regions of the brain, leaving the rest of the brain unexplored. Therefore, there is critical need to accurately quantify the level of class-lla HDACs expression in vivo in live humans to enable side-by-side comparison of the healthy brain to the disease state in the entire brain.

Despite being initially dismissed as histone deacetylases, recent studies in cancer and neurodegenerative diseases showed that overexpression of class-lla HDACs resulted in relative abundance of deacetylated histone (Lobera M M K, 2013; Yeh H H Y D, et al. 2013; Guerriero J L S A, 2017). Moreover, inhibition of class-lla HDACs restores histone acetylation status and improved outcome, which support class-lla HDACs as functional histone deacetylases. Notably, Guerriero et al. demonstrated that specific inhibition of class-lla HDACs in breast cancer restored the acetylation status of the core histone proteins and resulted in reduced primary tumor size and decreased pulmonary metastases thus highlighting the therapeutic value of class-lla HDACs (Guerriero J L S A, 2017; Cassetta L P J 2017). The deacetylation power of class-lla HDACs is still unclear. It could be explained by their overexpression (higher class-lla protein density increases histone deacetylation). Also, class-lla HDACs have been reported to recruit HDAC3 to form an enzymatically active multiprotein complex (class-lla HDAC-HDAC3-SMRT/N—CoR). In this model, the catalytic pocket of class-lla HDACs was essential for the deacetylase activity of this multiprotein complex (Fischle W D F, 2002). These studies suggest that the class-lla HDACs gain of function is through enzymatically active multiprotein complex and provide evidence for class-lla HDAC inhibition is a viable therapeutic target.

Molecular imaging with positron emission tomography (PET) is a non-invasive powerful tool that can provide quantitative and repeated measurements in real time on the class-lla HDAC protein expression. Currently, reliable class-lla HDAC PET tracers for imaging of cancer and the disorders of the brain are lacking. Therefore, the class-lla HDACs was targeted for PET tracer development to enable non-invasive in vivo visualization, detection and quantitative mapping of the biodistribution of class-lla HDAC protein expression in cancer and brain diseases. This invention developed PET tracer that detect brain disorders at an early point in time when therapeutic intervention is most effective and support and accelerate clinical development of specific class-lla HDACs targeting drugs. Also, PET tracer developed in the current invention further identifies a subset of patient population who may benefit the most from anti-class-lla therapy and monitor their response to treatment.

The expression levels and distribution of class-lla HDACs in human brain is largely unknown. The publicly available human protein atlas (HPA) database shows: (1) High HDAC-4 protein expression in the cerebral cortex and medium expression in the cerebellum, caudate and hippocampus; (2) moderate HDAC-5 protein expression in the cerebral cortex, cerebellum and hippocampus and low expression in the caudate; (3) no data available on the protein expression of HDAC-7 and (4) moderate HDAC-9 protein expression in the cerebral cortex and cerebellum, low expression in the caudate and no expression in the hippocampus. The publicly available database (GTEx portal) reports variable gene expression of class-lla HDACs in the brain (low gene expression of HDAC-4, 7 and 9 and moderate expression of HDAC-5 in the cortex, cerebellar hemisphere and cerebellum). However, these data obtained via semi-quantitative or qualitative measurement and current invention overcomes these limitations.

Despite intensive efforts by many research groups, radiolabeled HDAC inhibitors with either F-18 or C-11 PET radiotracers such as ¹⁸F-SAHA, ¹⁸F-FESAHA, and ¹¹C-MS-275 and other tracers showed poor blood-brain barrier (BBB) permeability and thus limited their utility for brain imaging (Hendricks, J_A., et al., 2011; Zeglis, B. M., et al., 2011; Hooker, J. M., et al., 2010; Seo, Y. J., et al., 2013).

¹¹C-martinostat was the first successful class-I HDAC (HDAC1, 2 and 3) inhibitor-based tracer (Wang, C., et al., 2014; Wey, H. Y., et al. 2015; Reid, A. E., et al., 2009). Also, a semi-selective HDAC6 PET imaging probe was reported (Wey H Y, G. T., Zurcher N R, et al. 2016). However, these probes do not provide information on the expression or distribution of class-lla HDACs or their involvement in cancer or CNS diseases. Moreover, radiolabeled class-lla HDACs substrates that undergo metabolism by class-lla enzymes to release ¹⁸F-fluorinated acetate in vitro and in vivo were previously reported (Bonomi, R., et al., 2015; Laws, M. T., et al., 2013; Guerriero, J. L., et al., 2017). However, these substrates exhibited poor in vivo pharmacokinetics that were detrimental to their clinical utility as PET radiotracers. An independent study that used ¹⁸F-FAHA (a substrate-type radiotracer) indicated that fluoroacetate produced in the brain does not remain localized to the point of catabolism of ¹⁸F-FAHA by class lla HDACs, thus confounding the PET signal and limits its practical utilization for quantitative imaging (Reid, A. E., et al., 2009). Therefore, a metabolically stable F-inhibitor-type radiotracer was pursued to overcome the inherent limitations of the labelled substrates, thus providing accurate quantification of class-lla HDACs protein expression by PET imaging.

TMP195 and CHDI-390576 were among the very few potent and highly specific class-lla HDAC inhibitors that were recently reported in the literature (FIG. 1) (Guerriero, J. L., et al., 2017).

CHDI-390576 is a second generation benzhydryl hydroxamic acid with improved CNS properties and selectivity to class-lla HDACs over the previously reported cyclopropane hydroxamic acids by the same group (Burli, R. W. et al., 2013). Although CHDI-390576 is fluorinated, the peculiar positioning of the fluorine atoms rendered this molecule likely inaccessible for facile F-18 incorporation. In contrast, TMP195 contains the 5-trifluoromethyl-1,2,4-oxadiazole (TFMO) moiety that in theory is accessible for late-stage radiolabeling with F-18. Therefore, a late stage radiochemical synthesis was designed in Scheme 1 (FIG. 3) to radiolabel the TFMO moiety with F-18 which validated the utility of this route to produce ¹⁸F-NT57, an identical radioactive homolog of TMP195. The utility of the new tracer was validated by performing in vivo PET imaging studies in healthy rats, thus mapping the biodistribution of TMP195/18F-NT57 in the tissues of the healthy rats.

### SUMMARY OF THE INVENTION

This invention provides a compound having the structure:

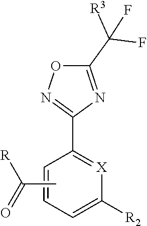

wherein:
X is C or N;
   R is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen;

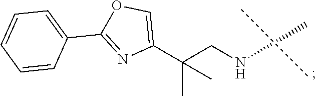

wherein when R² is H, R³ is F, X is N, and R and R² are in ortho position, R is other than

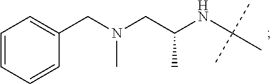

wherein when R² is F, R³ is F, X is C, and R and R² are in ortho position, R is other than

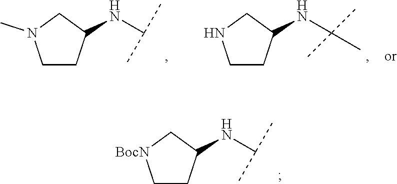

wherein when R² is H, R³ is F, X is C, and R and R² are in ortho position, R is other than OH or

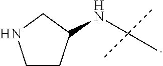

This invention provides a composition comprising compound A having the structure:

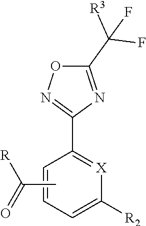

and
compound B having the structure

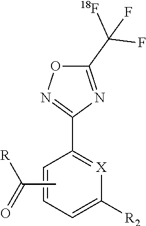

wherein:
X is C or N;
R is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen.

This invention provides a process for preparing compound III having the structure:

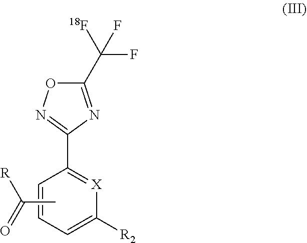

comprising
   a) reacting R—H with compound IIIa having the structure:

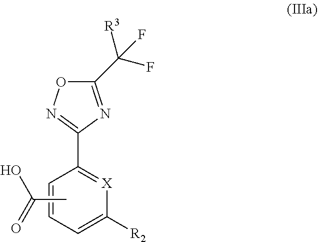

in 1-[Bis(dimethylamino)methylene]-1H-1,2,3-triazolo[4,5-b]pyridinium 3-oxid hexafluorophosphate (HATU), 4-methylmorpholine (NMM) and dimethylformamide (DMF) to obtain compound IIIb having the structure:

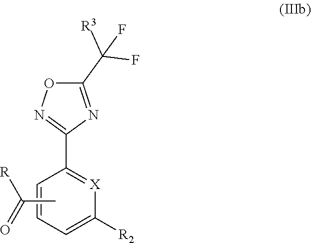

b) reacting compound IIIb with a [¹⁸F] fluorinating agent in a solvent to obtain compound III having structure:

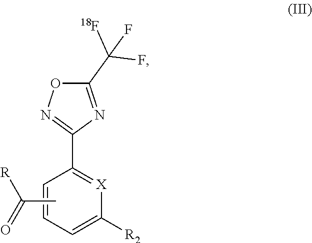

wherein:
X is C or N;
R is H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;

each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen.

This invention provides a process for preparing compound IV having the structure:

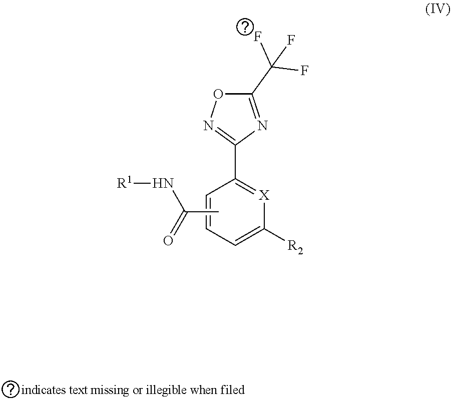

comprising:
   a) reacting compound I having the structure:

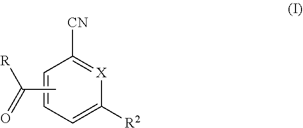

with hydroxylamine to obtain compound II having the structure:

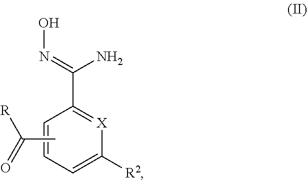

b) reacting compound II with bromodifluoroacetic anhydride in pyridine to produce the structure:

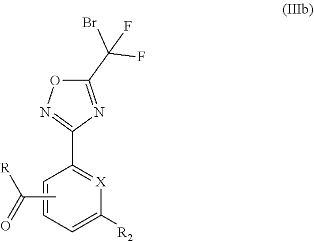

c) reacting compound IIIb with an amine in 1-[Bis(dimethylamino)methylene]-1H-1,2,3-triazolo[4,5-b]pyridinium 3-oxid hexafluorophosphate (HATU) and an excess amount of 4-methylmorpholine (NMM) to obtain compound IVb having the structure:

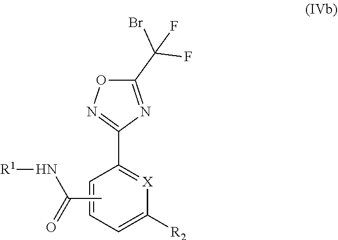

d) further reacting compound IVb with a [¹⁸F] fluorinating agent in a solvent to obtain compound IV having the structure:

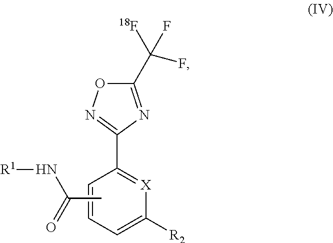

wherein:
X is C or N;
R is H, OH, O—C₁₋₆ alkyl, carbonyl, formyl or R¹;
R¹ is selected from the group consisting of H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl; and
R² is H or halogen.

This invention provides a method of detecting a disease of the central nervous system (CNS) in a subject, the method comprising:
a) administering a radiolabeled histone deacetylase (HDAC) inhibitor in a mammal having the structure:

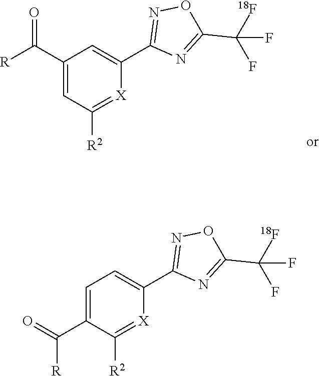

wherein:
X is CH or N;
   R is H, OH, —O—C₁₋₆ alkyl, carbonyl, formyl C₁₋₆alkyl, —C₁₋₆alkenyl, C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl; and
R² is H or halogen;
   b) detecting the presence of the radiolabeled HDAC inhibitor in the mammal using positron emission tomography (PET).

### BRIEF DESCRIPTION OF THE FIGURES

FIG. 1. Highly potent inhibitors of class-lla H DACs.

FIG. 2. General synthesis of bromodifluorooxadiazoles (bromo-Precursors).

FIG. 3. Scheme 1 of Radiosynthesis of ¹⁸F-TFMO containing amides.

FIG. 4. Scheme 2 of Radiosynthesis of ¹⁸F-TFMO containing esters and benzoic acids.

FIG. 5. Scheme 3 of Radiosynthesis of [¹⁸F]TMP195 (¹⁸F-NT57).

FIG. 6. High-pressure liquid chromatography (HPLC) chromatograms (a) radioactive peak of ¹⁸F-NT57 detected with radioactivity detector, (b) ¹⁸F-NT57 associated clod mass was detected with ultraviolet detector and c) TMP195 detected with ultraviolet detector. ¹⁸F-NT57 peaks detected by both ultraviolet (254 nM) and radioactive traces were consistent with TMP195 ultraviolet peak thus confirming the identity of the target tracer.

FIG. 7. Maximum projection intensity (MIP) images (coronal) summed over 15 min each. Tracer uptake A): a) harderian, b) wrist, c) cervical, d) interscapular tissues; B) blocking with a cold dose of TMP195. C) Time activity curve of ¹⁸F-NT57 in tissues obtained from dynamic imaging for 60 min. D) Blocking studies with TMP195 (baseline vs treated) at 60 min post injection.

FIG. 8. A) Coronal slices of rat brain obtained from summed PET images (0-15 min). B) Time activity curve at the baseline and after treatment with a cold dose of TMP195 (self-blocking).

FIG. 9. Scheme A—General synthesis of non-radioactive standard (Class-IIa HDAC inhibitors) and precursors for radiolabeling (examples in table 1 and 2). X═Br, F, Cl, I, hydrocarbon. Y═C, N.

FIG. 10. A) Radiosynthesis of [¹⁸F]-NT2-160, a highly potent class-lla HDAC inhibitor. B) Representative PET images of rat brain obtained with [¹⁸F]-NT2-160 demonstrating high quality images and heterogenous uptake. C) Time-activity curves in rat brain (whole), heart and muscle; and D) Time-activity curves in rat brain regions obtained from dynamic imaging for 60 mins. Cx: cortex; TH: Thalamus; HC: hippocampus and Cer: cerebellum.

FIG. 11. Representative PET/CT image with ¹⁸F-NT160. A) Pet image of rat brain, B) Fusion PET/CT image (Coronal), C) Fusion PET/CT image (sagittal) and D) Time activity curve of ¹⁸F-NT160 in rat brain obtained from dynamic imaging over 60 min. Crt: Cortex, Cer: cerebellum, Th: thalamus, Hip: hippocampus.

### DETAILED DESCRIPTION OF THE INVENTION

The following detailed description of embodiments of the invention will be made in reference to the accompanying drawings. In describing the invention, explanation about related functions or constructions known in the art are omitted for the sake of clearness in understanding the concept of the invention to avoid obscuring the invention with unnecessary detail.

Embodiments of the present invention described herein provide radiolabeled compositions and methods of use thereof. The compositions can be used for imaging of disorders of the central nervous system for the use in early detection of diseases including memory and cognitive impairment, dementia and behavioral changes, Alzheimer's and Huntington diseases and others.

In some embodiments, the present invention provides a compound having the structure:

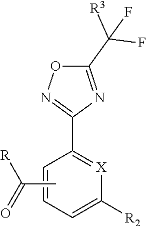

wherein:
X is C or N;
   R is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and R³ is halogen;
   wherein when R² is H, R³ is F, X is C, and R and R² are in meta position, R is other than

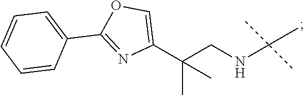

wherein when R² is H, R³ is F, X is N, and R and R² are in ortho position, R is other than

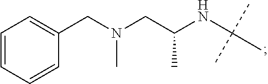

wherein when R² is F, R³ is F, X is C, and R and R² are in ortho position, R is other than

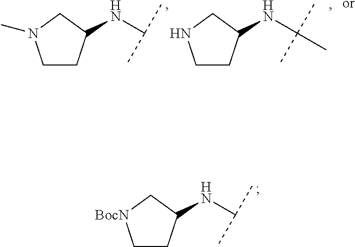

wherein when R² is H, R³ is F, X is C, and R and R² are in ortho position, R is other than OH or

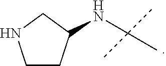

In some embodiments, R is NH—R¹ wherein R¹ is C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-heteroaryl-aryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl.

In some embodiments, R is —NH—R¹ wherein R¹ is C₁₋₆alkylC(O)NC₁₋₆alkyl phenyl, C₁₋₆alkyl-N—C₁₋₆alkyl phenyl, C₁₋₆alkyl-N-oxazole-phenyl, C₁₋₆alkyl-oxazole-phenyl, C₁₋₆alkylC(O)—C₁₋₆alkyl-NH—C₁₋₆alkyl phenyl, pyrrolidine, pyrrolidine phenyl, C₁₋₆alkyl-pyrrolidine, C₁₋₆alkyl-pyrrolidine-phenyl, C₁₋₆alkylC(O)-pyrrolidine, C₁₋₆alkylC(O)-pyrrolidine-phenyl, piperidine, piperidine phenyl, C₁₋₆alkyl-piperidine, piperidine-C₁₋₆alkyl-phenyl, C₁₋₆alkylC(O)-piperidine, C₁₋₆alkylC(O)-piperidine-phenyl.

In some embodiments, R is substituted aryl, heteroaryl, cycloalkyl, heterocyloalkyl, wherein the substitution is OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, and —C₀₋₆alkylNHC₀₋₆alkyl heterocyloalkyl, each of which is optionally substituted by one or more substituents selected from —C₀₋₆alkyl, —C₁₋₆alkylOC₁₋₆alkyl, halogen, or —C(O)OtButyl.

In some embodiments, when R² is H, R³ is F, X is C, and R and R² are in meta position, R is other than

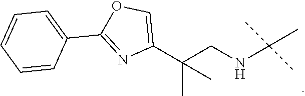

In some embodiments, when R² is H, R³ is F, X is N, and R and R² are in ortho position, R is other than

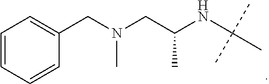

In some embodiments, when R² is F, R³ is F, X is C, and R and R² are in ortho position, R is other than

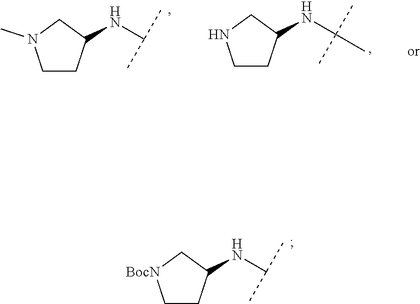

In some embodiments, wherein when R² is H, R³ is F, X is C, and R and R² are in ortho position, R is other than OH or

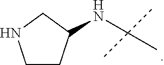

In some embodiments, R³ is F, Cl, Br or I.

In some embodiments, R³ is radioactive.

In some embodiments, the present invention provides a compound having the structure:

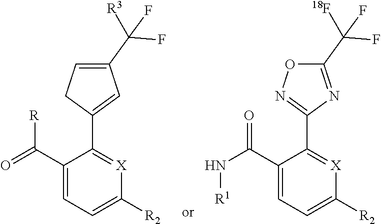

wherein when R² is H, R³ is F, X is C, and R and R² are in meta position, R is other than

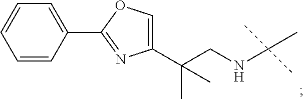

wherein when R² is H, R³ is F, X is N, and R and R² are in ortho position, R is other than

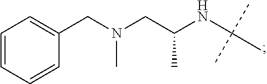

wherein when R² is F, R³ is F, X is C, and R and R² are in ortho position, R is other than

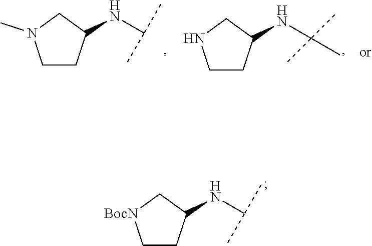

wherein when R² is H, R³ is F, X is C, and R and R² are in ortho position, R is other than OH or

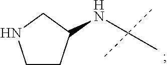

wherein when R¹ is H, R³ is F, X is C, and R and R² are in meta position, R is other than

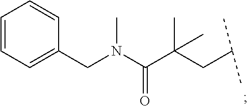

wherein when R¹ is H, R³ is F, X is N, and R and R² are in ortho position, R is other than

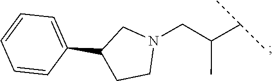

wherein when R¹ is F, R³ is F, X is C, and R and R² are in ortho position, R is other than

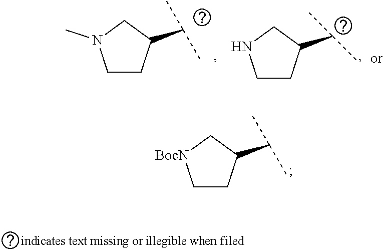

In some embodiments, when R¹ is H, R³ is F, X is C, and R and R² are in ortho position, R is other than OH or

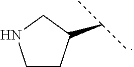

In some embodiments, when R¹ is H, R³ is F, X is C, and R and R² are in meta position, R is other than

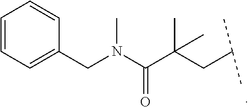

In some embodiments, when R¹ is H, R³ is F, X is N, and R and R² are in ortho position, R is other than

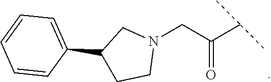

In some embodiments, when R¹ is F, R³ is F, X is C, and R and R² are in ortho position, R is other than

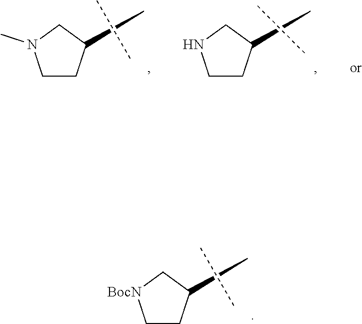

In some embodiments, when R¹ is H, R³ is F, X is C, and R and R² are in ortho position, R is other than OH or

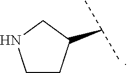

In some embodiments, the present invention provides a compound having the structure:

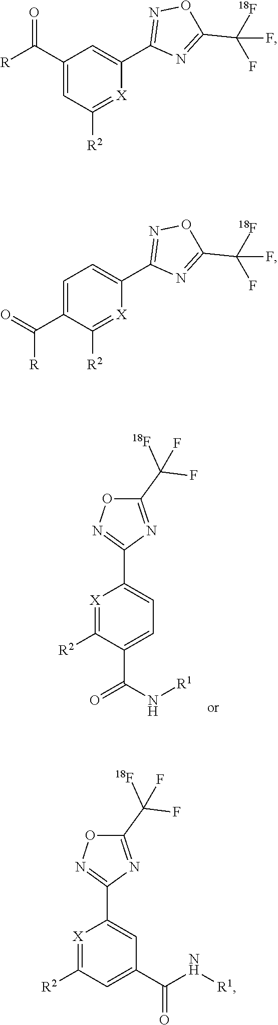

X is C or N;
R¹ is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen.

In some embodiments, R or R¹ is C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-heteroaryl-aryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl.

In some embodiments, R or R¹ is C₁₋₆alkylC(O)NC₁₋₆alkyl phenyl, C₁₋₆alkyl-N—C₁₋₆alkyl phenyl, C₁₋₆alkyl-N-oxazole-phenyl, C₁₋₆alkyl-oxazole-phenyl, C₁₋₆alkylC(O)—C₁₋₆alkyl-NH—C₁₋₆alkyl phenyl, pyrrolidine, pyrrolidine phenyl, C₁₋₆alkyl-pyrrolidine, C₁₋₆alkyl-pyrrolidine-phenyl, C₁₋₆alkylC(O)-pyrrolidine, C₁₋₆alkylC(O)-pyrrolidine-phenyl, piperidine, piperidine phenyl, C₁₋₆alkyl-piperidine, piperidine-C₁₋₆alkyl-phenyl, C₁₋₆alkylC(O)-piperidine, C₁₋₆alkylC(O)-piperidine-phenyl.

In some embodiments, oxazole, phenyl, pyrrolidine and piperidine is further substituted with halogen, C₁₋₆alkyl, aryl, or C₁₋₆alkyl aryl.

In some embodiments, halogen is F, Cl, Br, I.

In some embodiments, R or R¹ is further substituted with OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, and —C₀₋₆alkylNHC₀₋₆alkyl heterocyloalkyl, each of which is optionally substituted by one or more substituents selected from —C₀₋₆alkyl, —C₁₋₆alkylOC₁₋₆alkyl, halogen, or —C(O)OtButyl.

In some embodiments, R or R¹ is H, OH, methyl, formyl, or carbonyl.

In some embodiments, R or R¹ is OH.

In some embodiments, R or R¹ is methyl.

In some embodiments, R or R¹ is formyl, or carbonyl.

In some embodiments, R or R¹ is —O—C₁₋₆ alkyl.

In some embodiments, R or R¹ is —O-methyl, —O-ethyl or —O-tert-butyl.

In some embodiments, R² is H, F, Cl, Br or I.

In some embodiments, R² is H.

In some embodiments, R² is F.

In some embodiments, X is C.

In some embodiments, X is N.

In some embodiments, R or R¹ contains a radioactive label.

In some embodiments, R or R¹ contains a radioactive halogen.

In some embodiments, R or R¹ contains a ¹⁸F.

In some embodiments, R or R¹ contains a radioactive carbon.

In some embodiments, R or R¹ contains a ¹¹C.

In some embodiments, R is NH—R¹.

In some embodiments, R¹ is selected form the group consisting of:

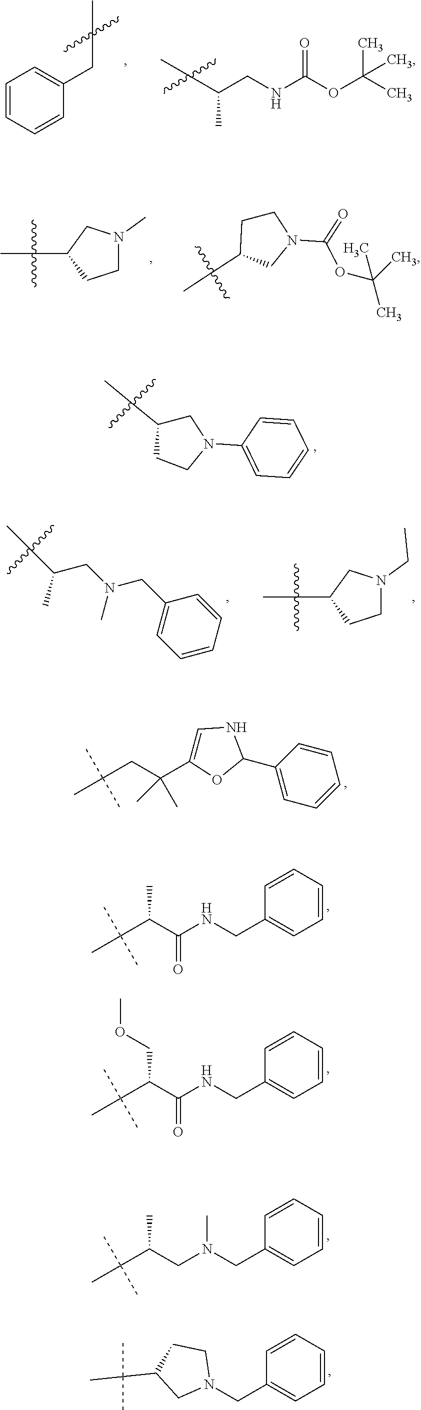

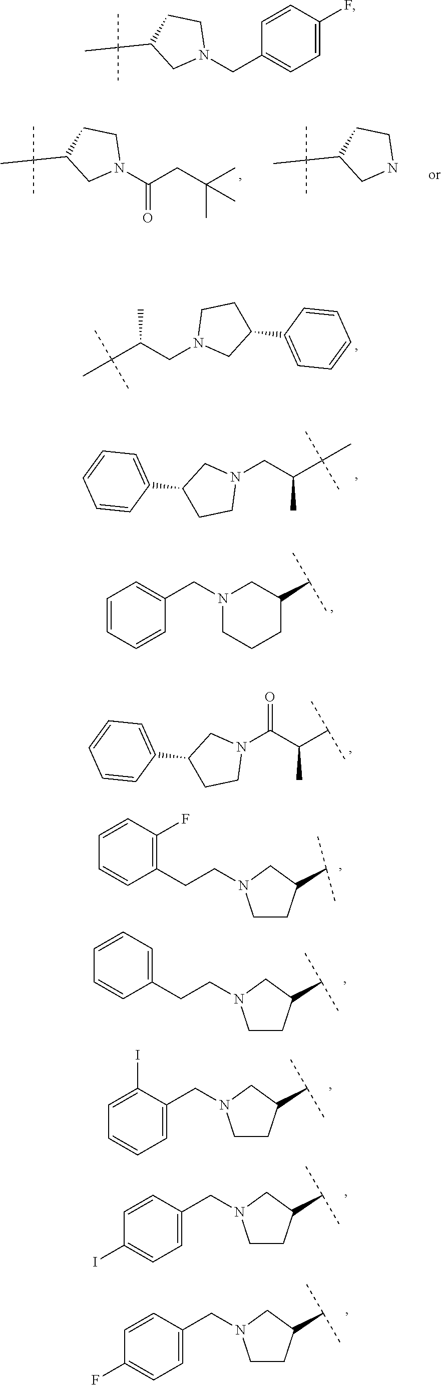

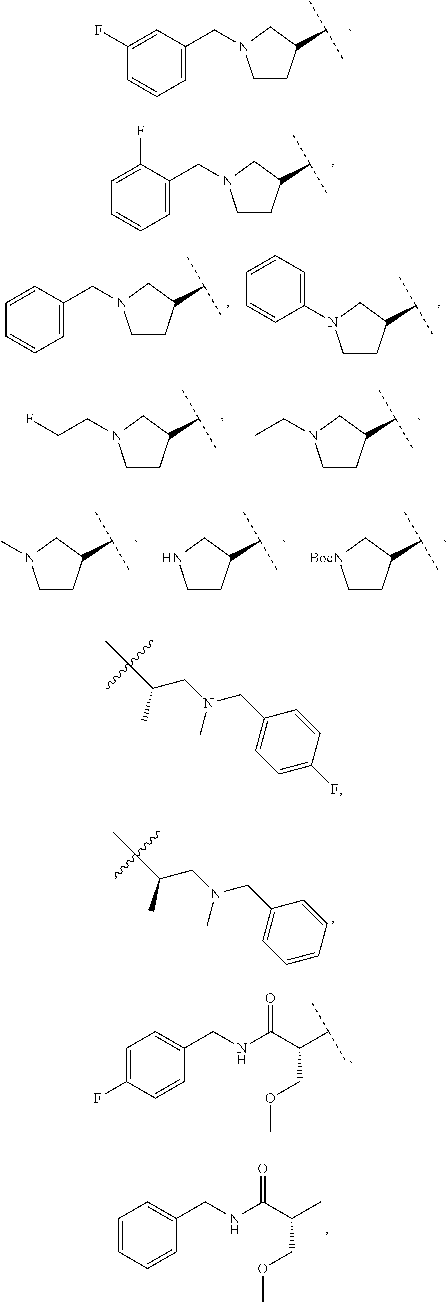

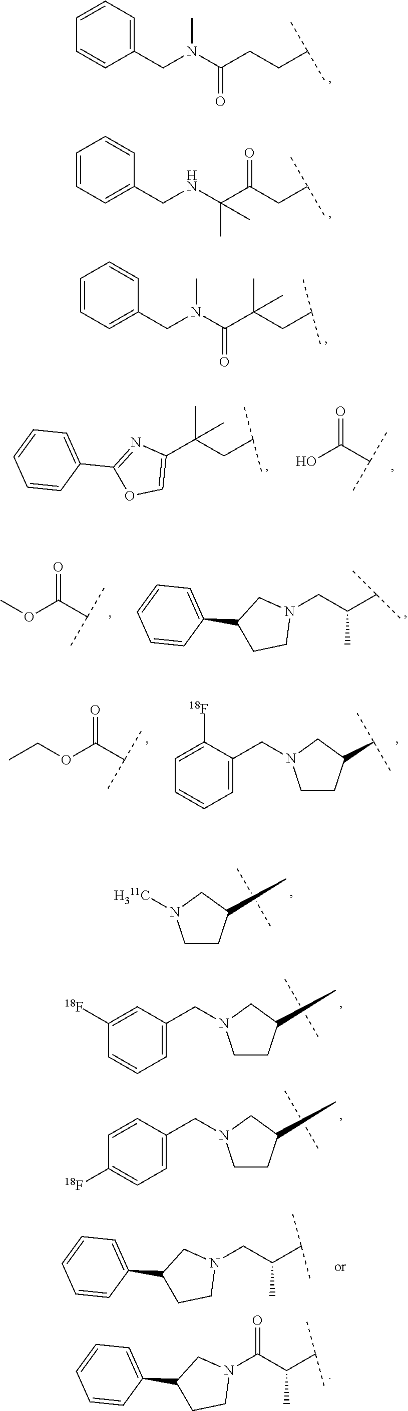

In some embodiments, the present invention provides a compound having the structure:

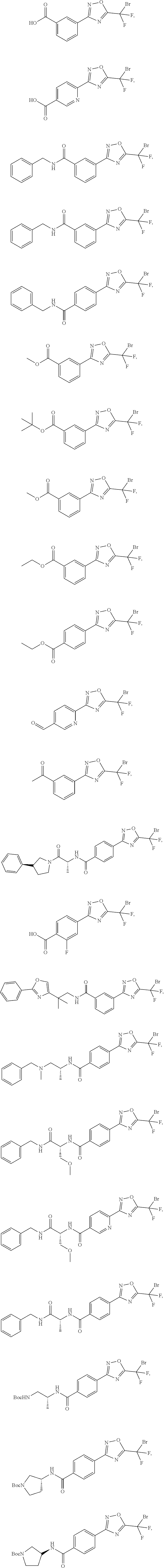

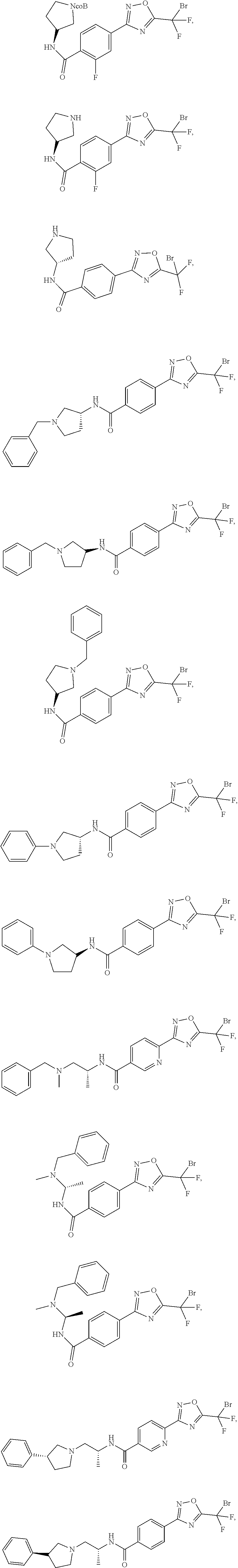

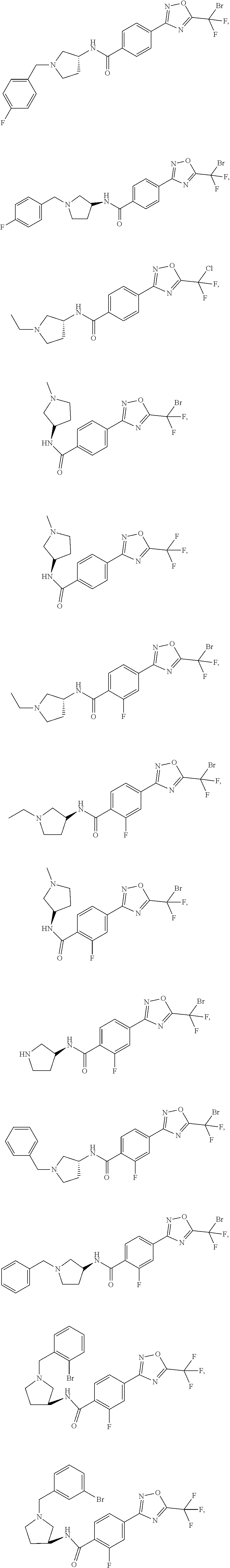

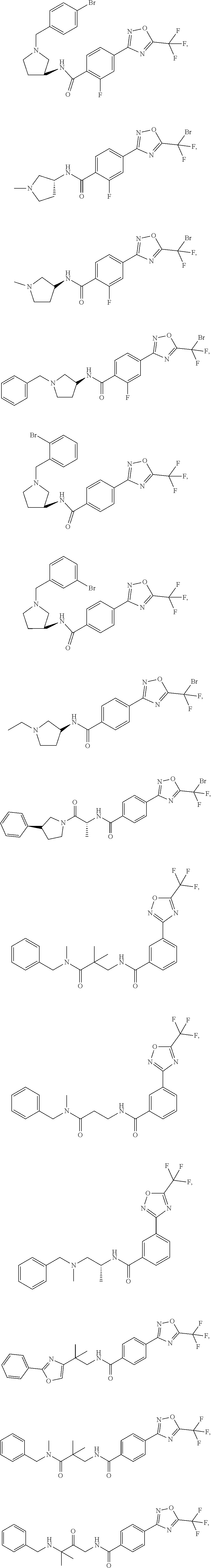

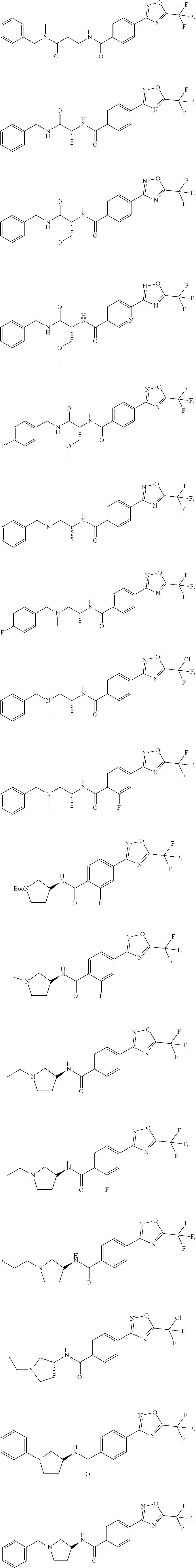

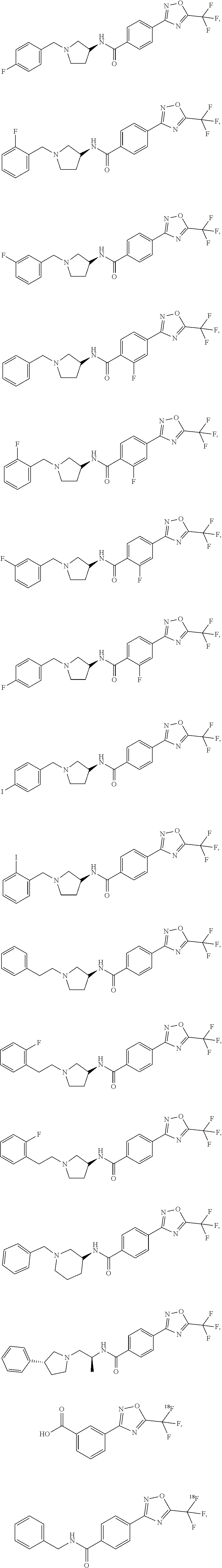

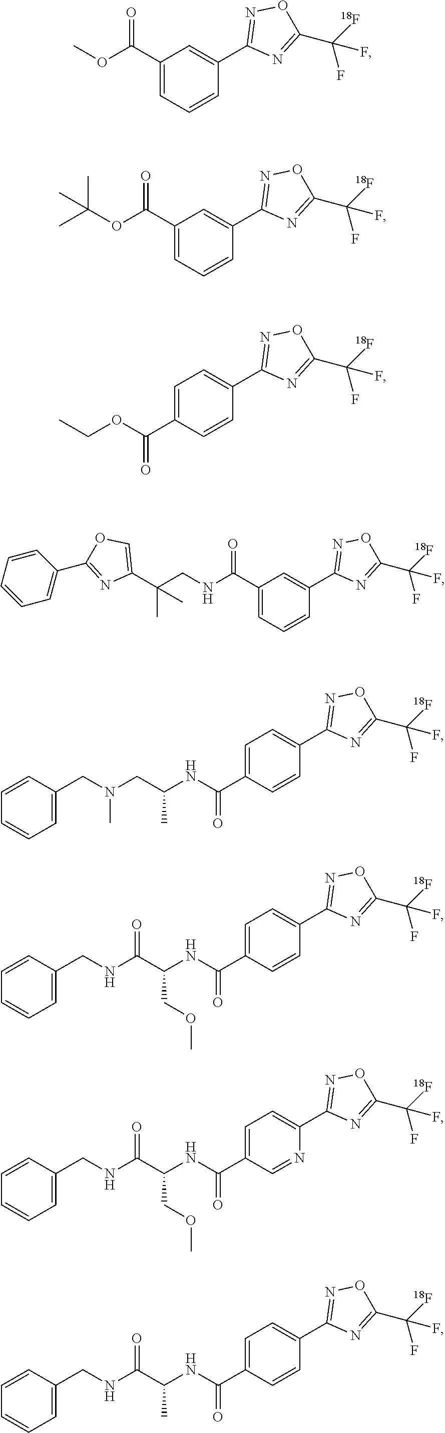

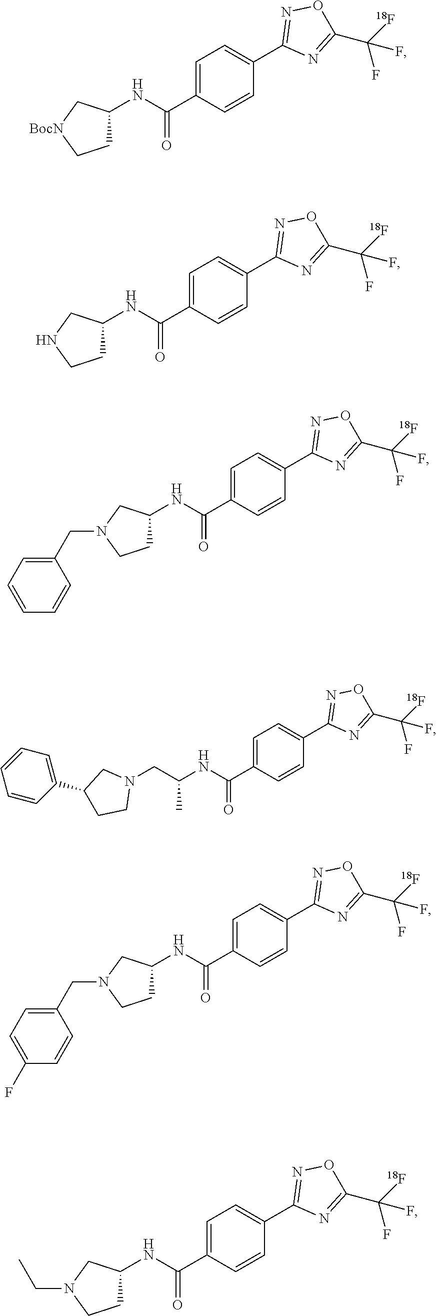 [SMILES: CC(C)(C)OC(=O)N1CC[C@@H](NC(=O)c2ccc(-c3noc(C(F)(F)[18F])n3)cc2)C1 ; O=C(N[C@@H]1CCNC1)c1ccc(-c2noc(C(F)(F)[18F])n2)cc1 ; O=C(N[C@@H]1CCN(Cc2ccccc2)C1)c1ccc(-c2noc(C(F)(F)[18F])n2)cc1 ; C[C@H](CN1CC[C@H](c2ccccc2)C1)NC(=O)c1ccc(-c2noc(C(F)(F)[18F])n2)cc1 ; O=C(N[C@@H]1CCN(Cc2ccc(F)cc2)C1)c1ccc(-c2noc(C(F)(F)[18F])n2)cc1 ; CCN1CC[C@@H](NC(=O)c2ccc(-c3noc(C(F)(F)[18F])n3)cc2)C1]

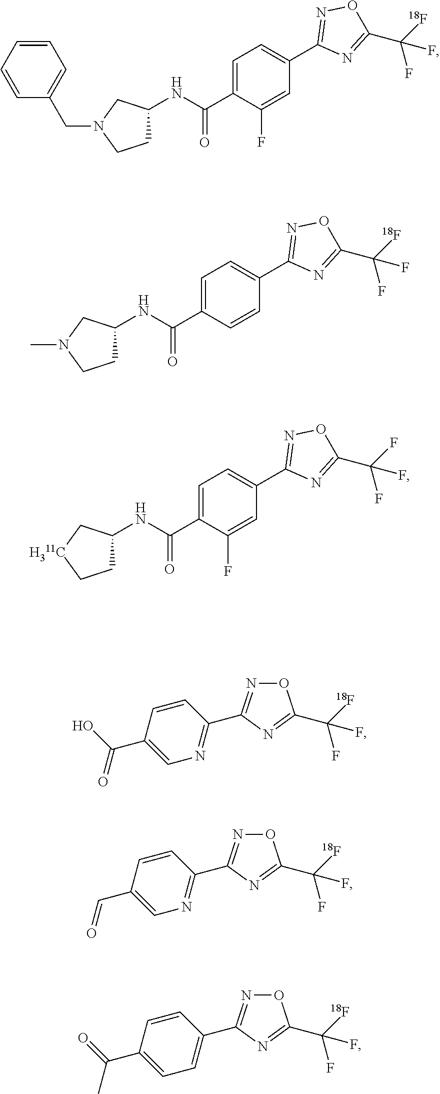 [SMILES: O=C(N[C@@H]1CCN(Cc2ccccc2)C1)c1ccc(-c2noc(C(F)(F)[18F])n2)cc1F ; CN1CC[C@@H](NC(=O)c2ccc(-c3noc(C(F)(F)[18F])n3)cc2)C1 ; O=C(N[C@@H]1CC[11CH2]C1)c1ccc(-c2noc(C(F)(F)F)n2)cc1F ; O=C(O)c1ccc(-c2noc(C(F)(F)[18F])n2)nc1 ; O=Cc1ccc(-c2noc(C(F)(F)[18F])n2)nc1 ; CC(=O)c1ccc(-c2noc(C(F)(F)[18F])n2)cc1]

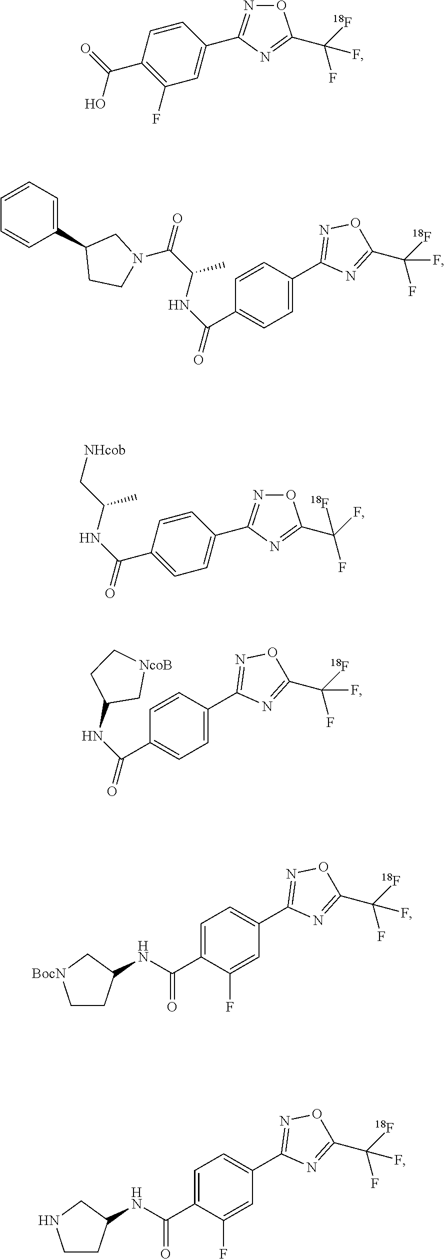

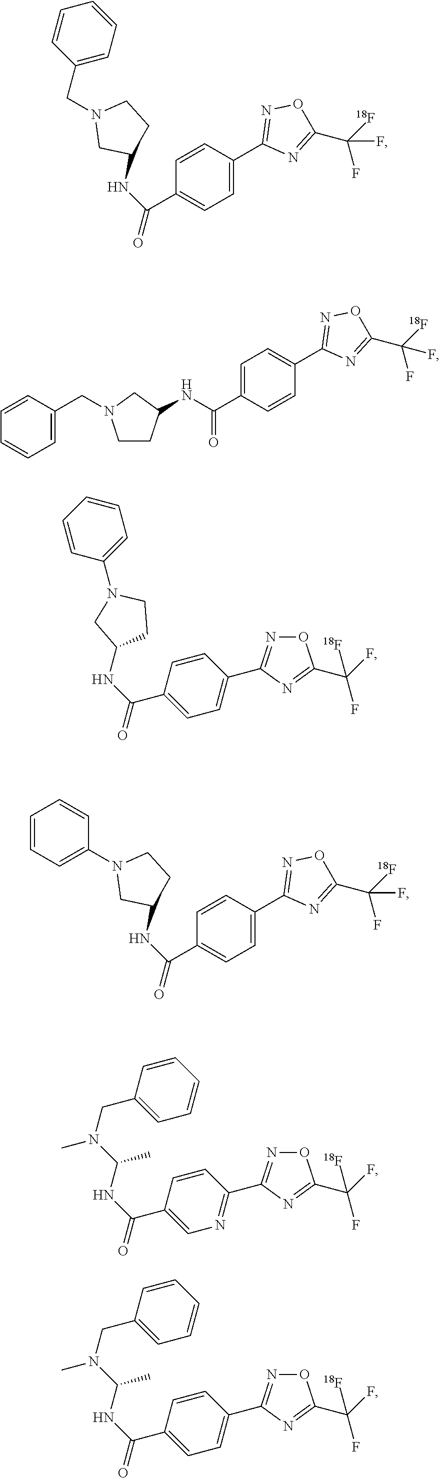

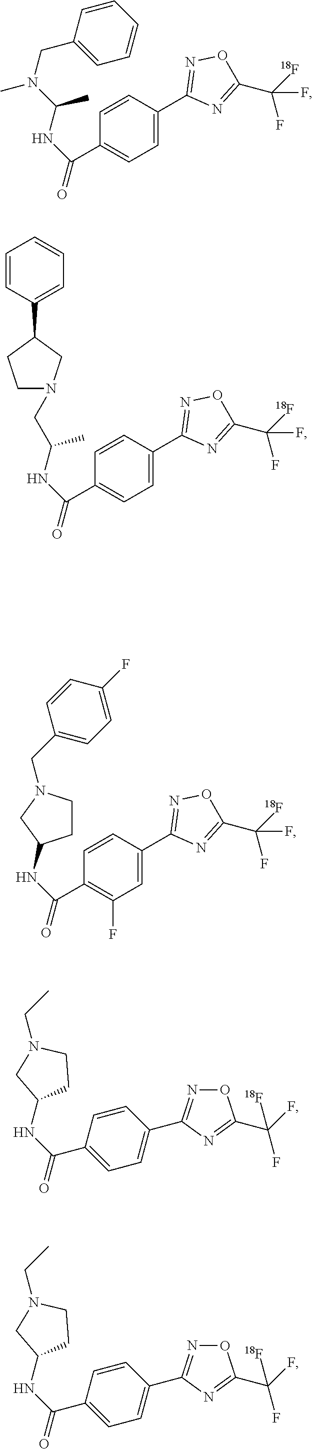

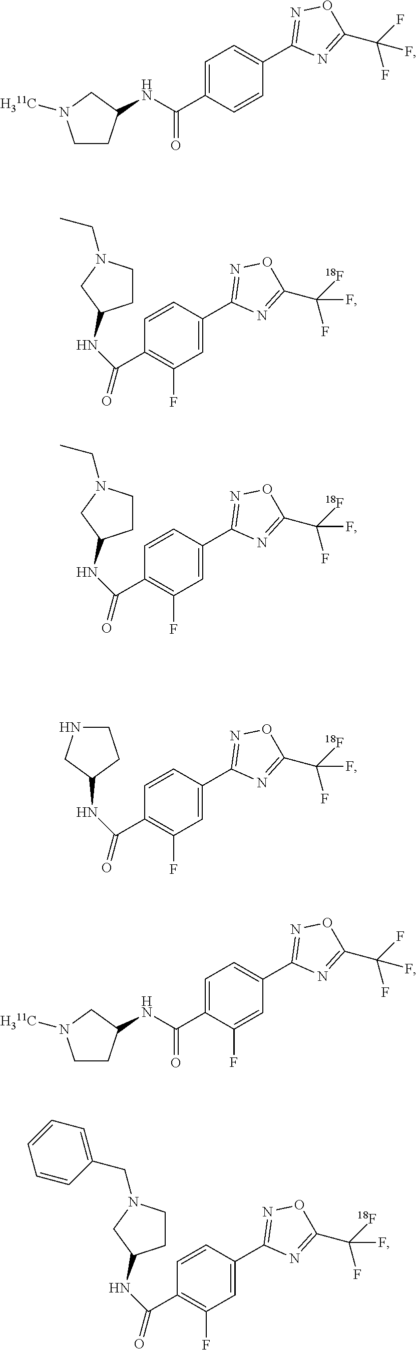

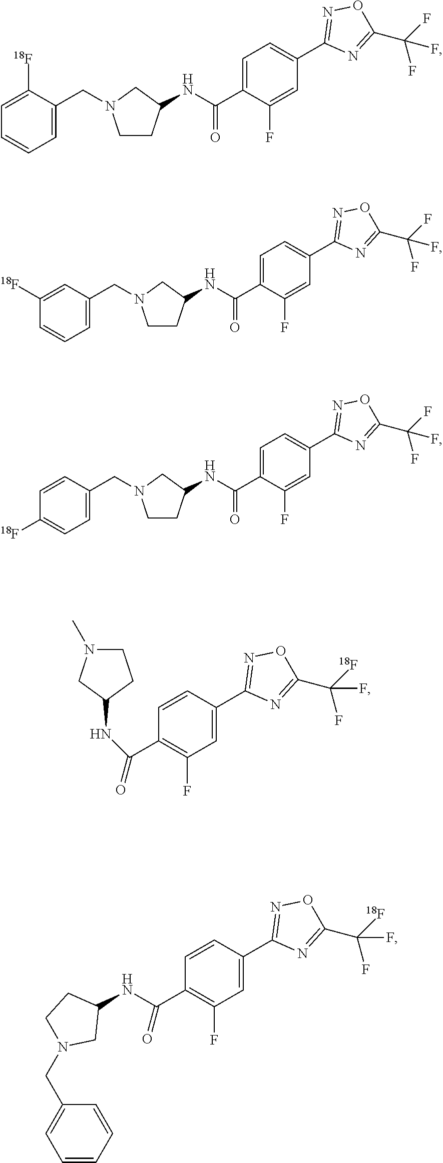

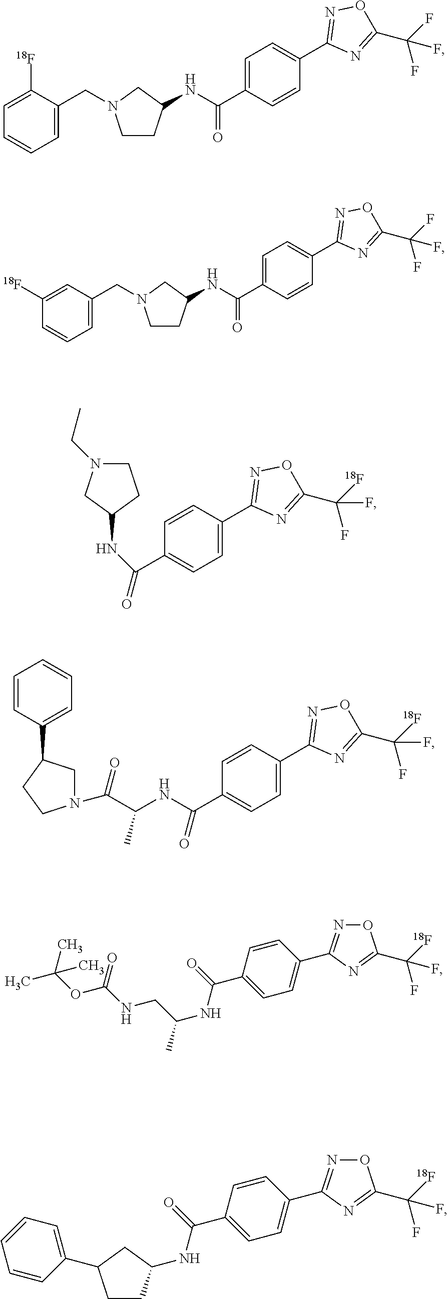

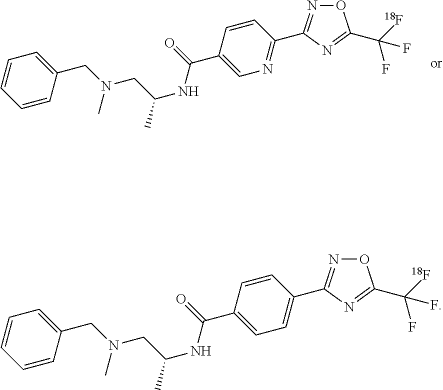 [SMILES: C[C@H](CN(C)Cc1ccccc1)NC(=O)c1ccc(-c2noc(C(F)(F)[18F])n2)nc1 ; C[C@H](CN(C)Cc1ccccc1)NC(=O)c1ccc(-c2noc(C(F)(F)[18F])n2)cc1]

In some embodiments, the present invention provides compounds that are human histone deacetylases (HDACs) inhibitors.

In some embodiments, the HDACs are HDAC-4, HDAC-5, HDAC-7 and HDAC-9.

In some embodiments, the HDACs inhibitors have the following structures:

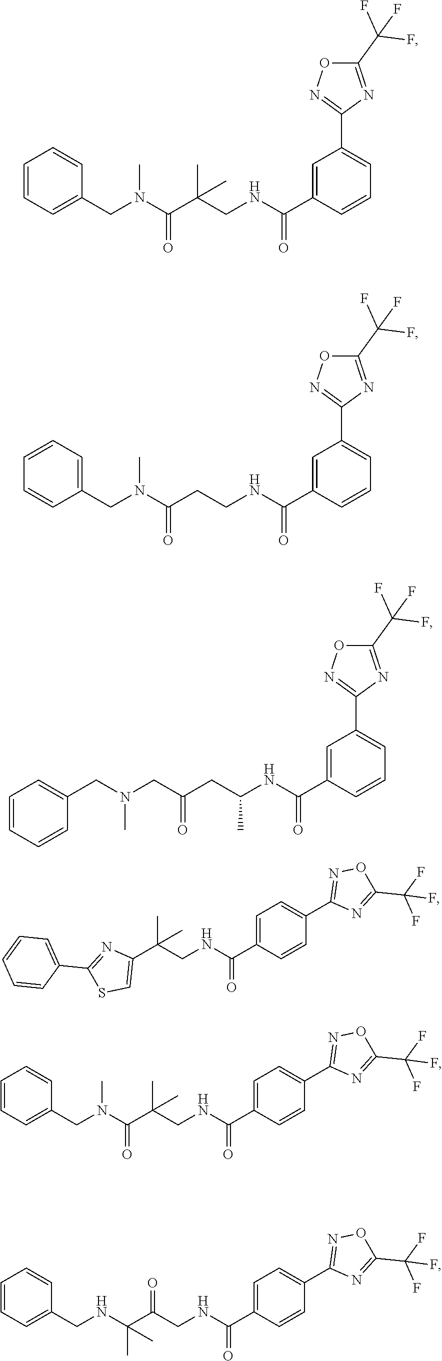 [SMILES: CN(Cc1ccccc1)C(=O)C(C)(C)CNC(=O)c1cccc(-c2noc(C(F)(F)F)n2)c1 ; CN(Cc1ccccc1)C(=O)CCNC(=O)c1cccc(-c2noc(C(F)(F)F)n2)c1 ; C[C@H](CC(=O)CN(C)Cc1ccccc1)NC(=O)c1cccc(-c2noc(C(F)(F)F)n2)c1 ; CC(C)(CNC(=O)c1ccc(-c2noc(C(F)(F)F)n2)cc1)c1csc(-c2ccccc2)n1 ; CN(Cc1ccccc1)C(=O)C(C)(C)CNC(=O)c1ccc(-c2noc(C(F)(F)F)n2)cc1 ; CC(C)(NCc1ccccc1)C(=O)CNC(=O)c1ccc(-c2noc(C(F)(F)F)n2)cc1]

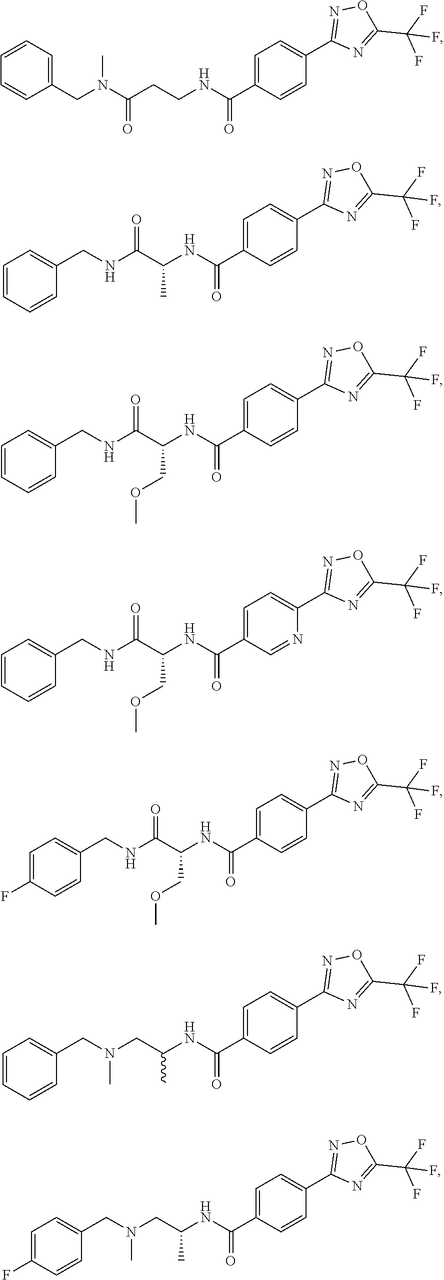

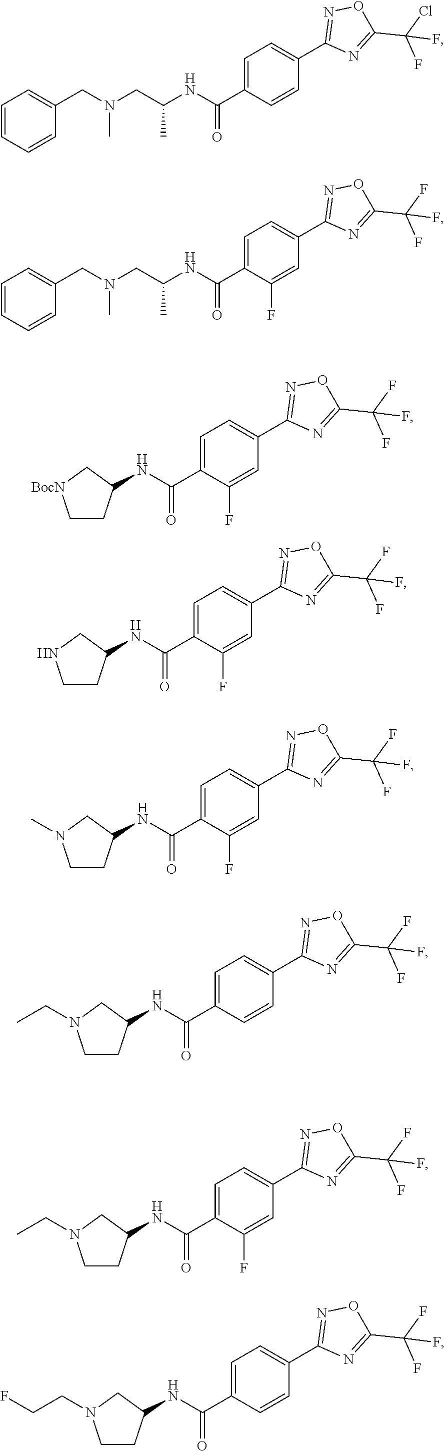

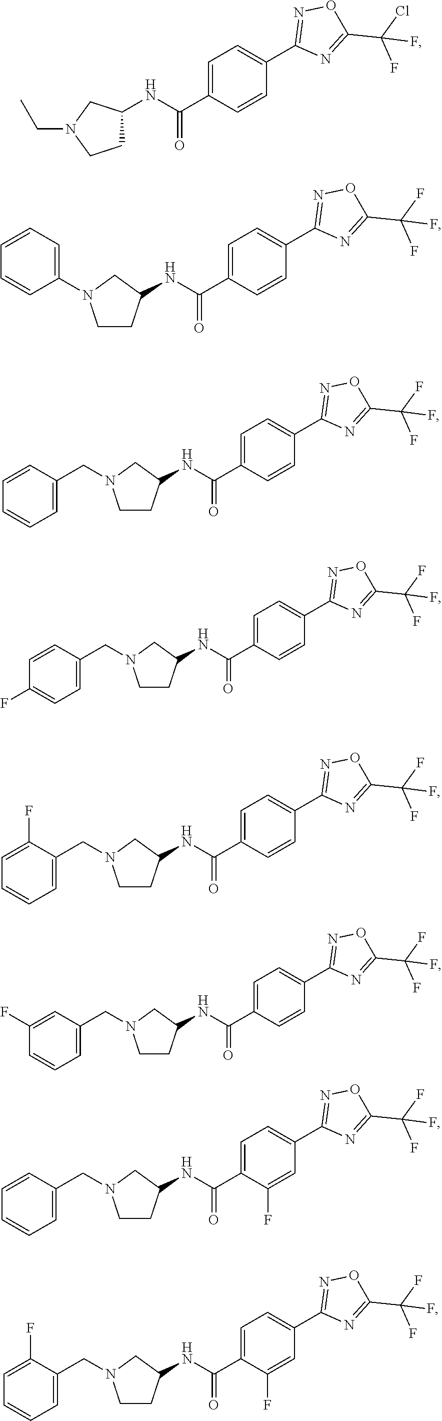

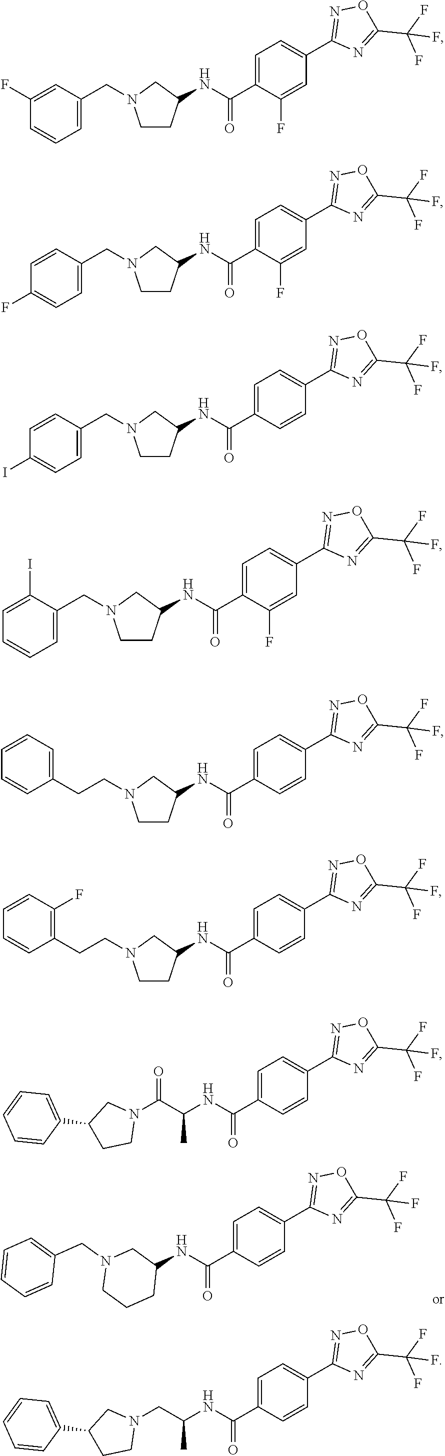

In some embodiments, the precursor used for radiolabeling has the following structure:

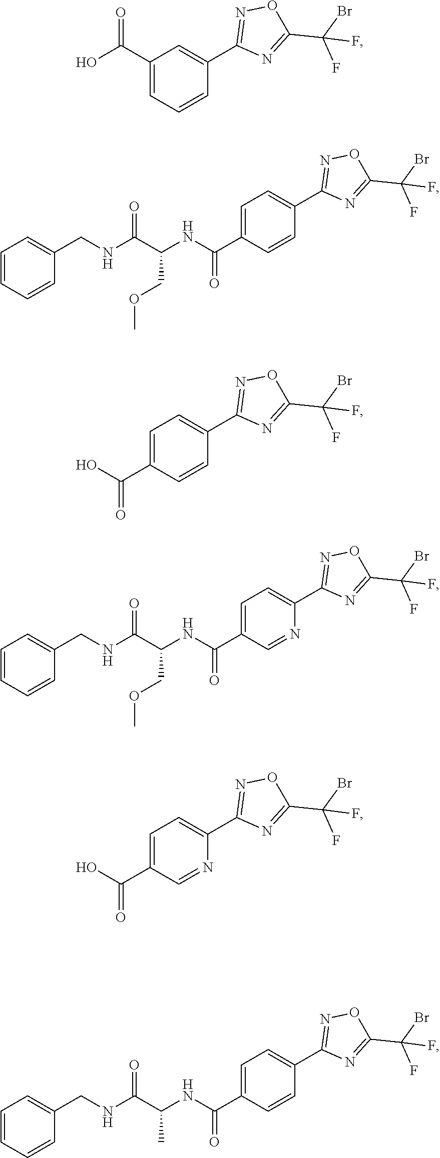 [SMILES: O=C(O)c1cccc(-c2noc(C(F)(F)Br)n2)c1 ; COC[C@@H](NC(=O)c1ccc(-c2noc(C(F)(F)Br)n2)cc1)C(=O)NCc1ccccc1 ; O=C(O)c1ccc(-c2noc(C(F)(F)Br)n2)cc1 ; COC[C@@H](NC(=O)c1ccc(-c2noc(C(F)(F)Br)n2)nc1)C(=O)NCc1ccccc1 ; O=C(O)c1ccc(-c2noc(C(F)(F)Br)n2)nc1 ; C[C@@H](NC(=O)c1ccc(-c2noc(C(F)(F)Br)n2)cc1)C(=O)NCc1ccccc1]

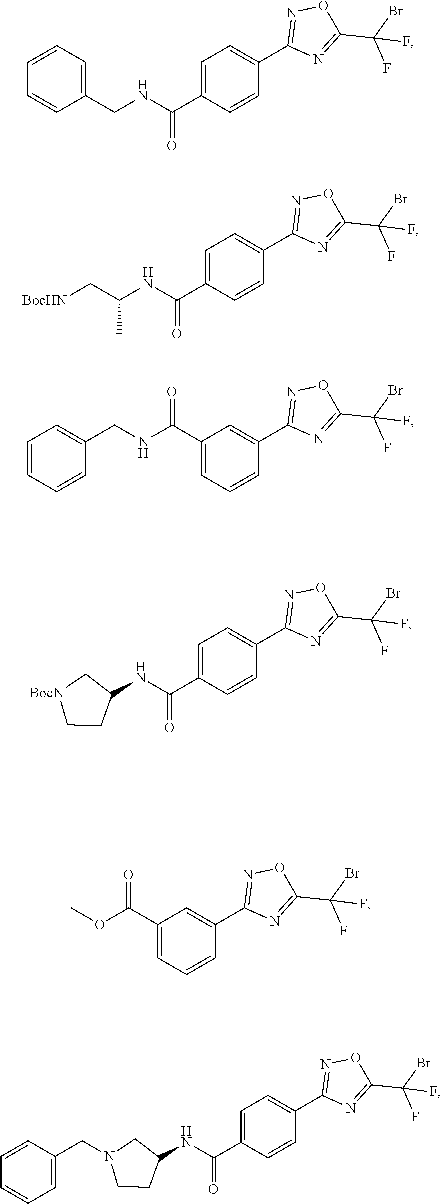

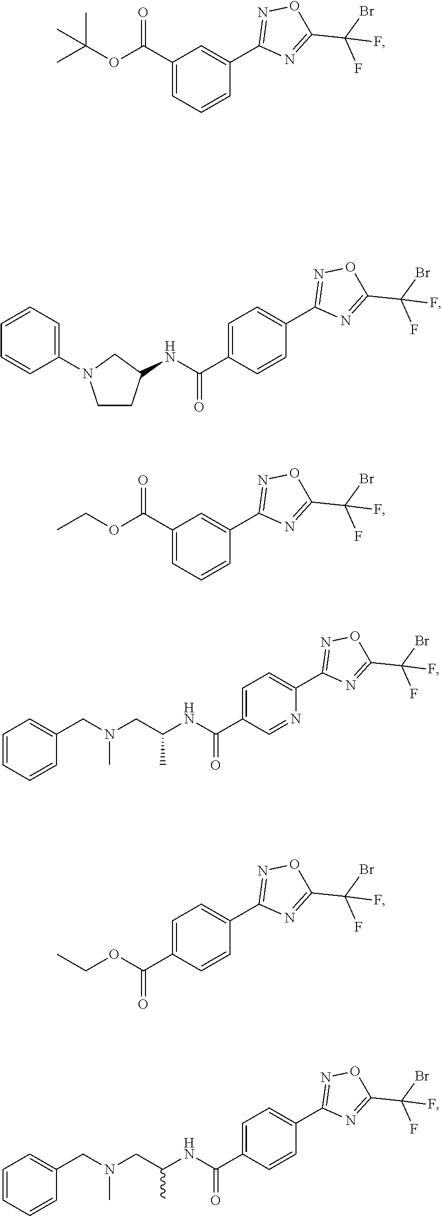

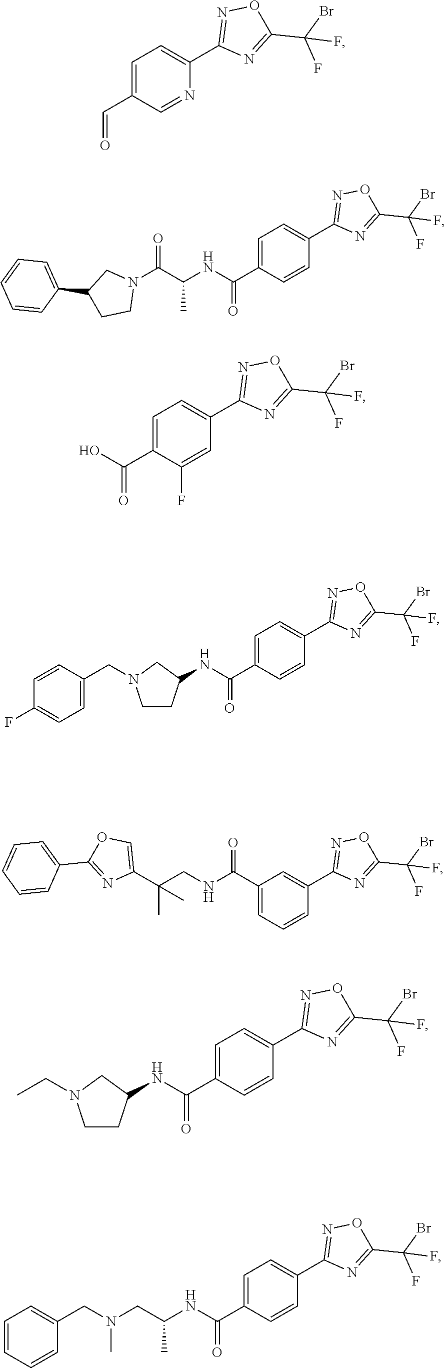

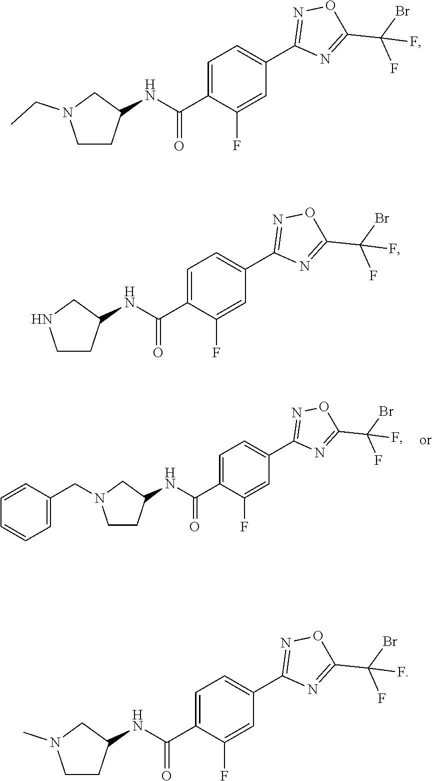

In some embodiments, the radioactive tracer has the following structure:

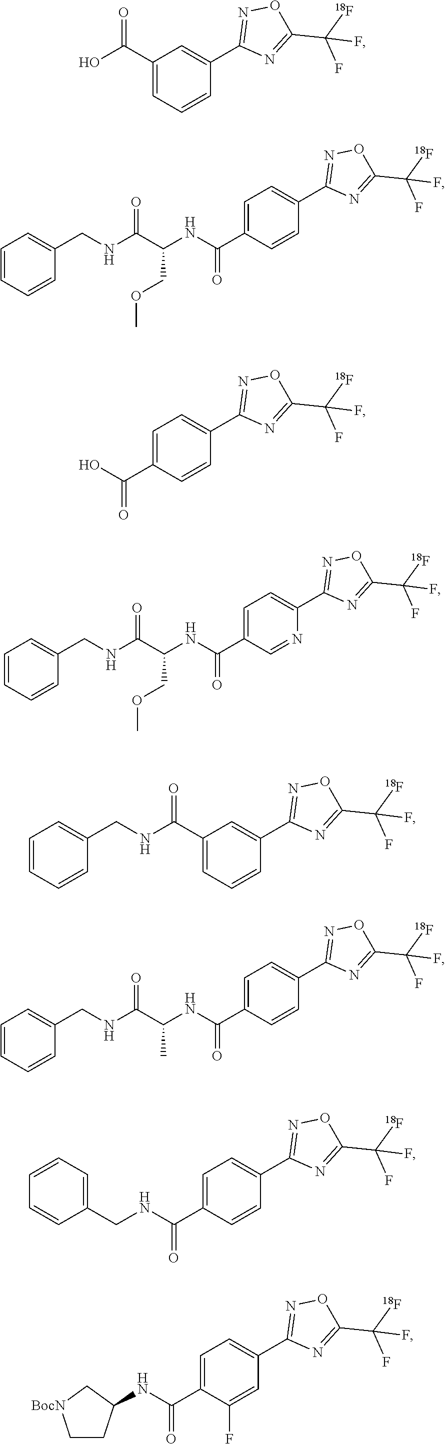

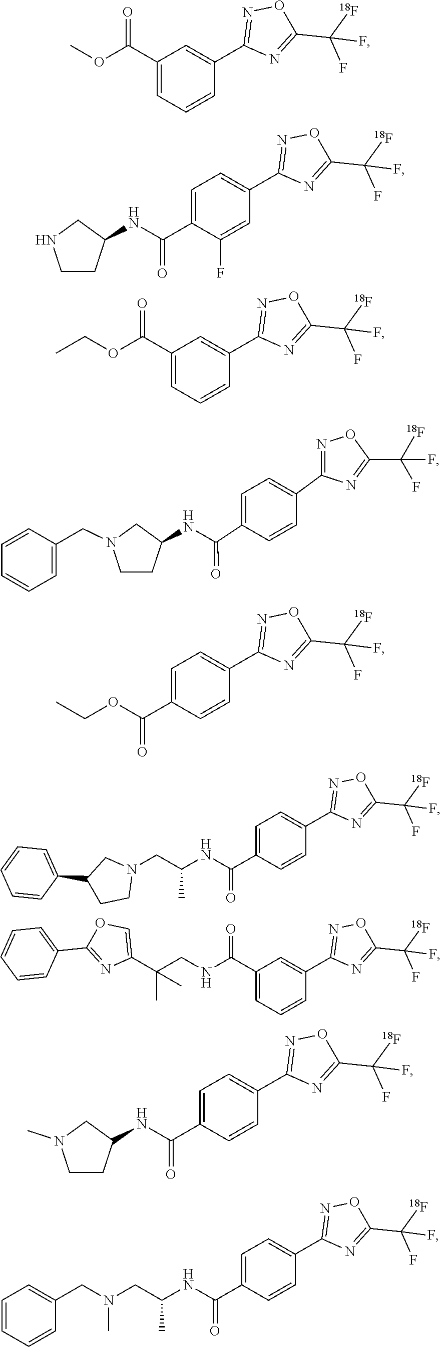

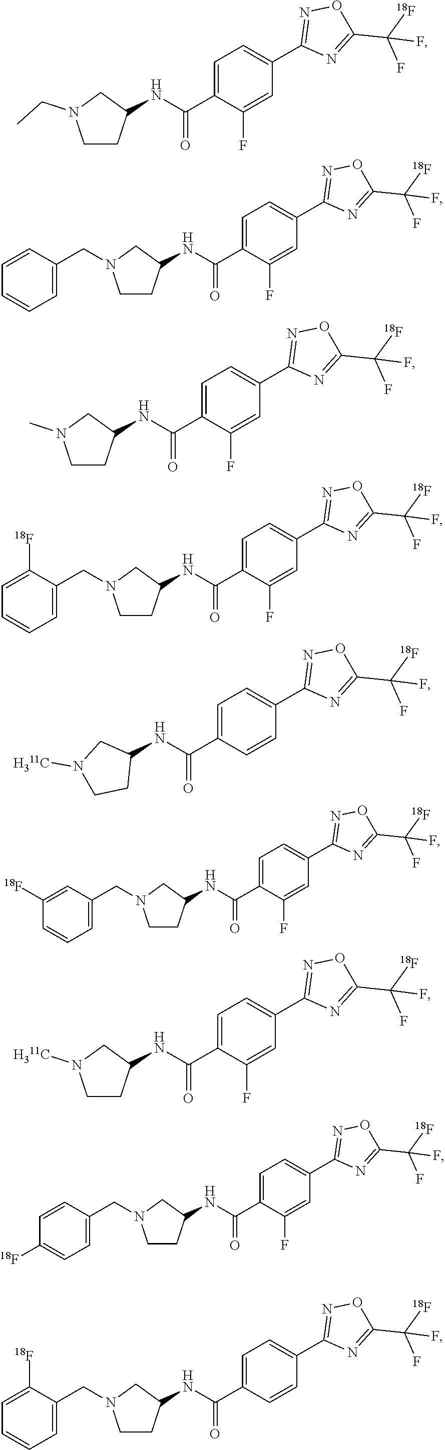

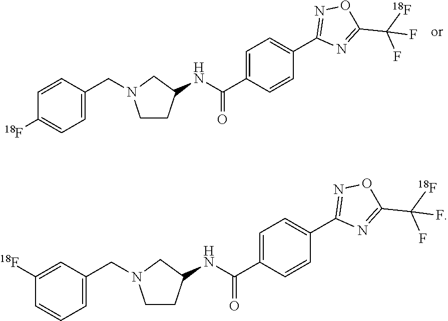

In some embodiments, the present invention provides a composition comprising compound A having the structure

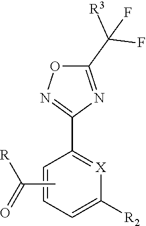

and
compound B having the structure

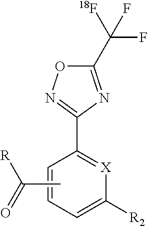

wherein:
X is C or N;
R is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen;
R³ is halogen.

In some embodiments, R or R¹ is C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-heteroaryl-aryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl.

In some embodiments, R or R¹ is C₁₋₆alkylC(O)NC₁₋₆alkyl phenyl, C₁₋₆alkyl-N—C₁₋₆alkyl phenyl, C₁₋₆alkyl-N-oxazole-phenyl, C₁₋₆alkyl-oxazole-phenyl, C₁₋₆alkylC(O)—C₁₋₆alkyl-NH—C₁₋₆alkyl phenyl, pyrrolidine, pyrrolidine phenyl, C₁₋₆alkyl-pyrrolidine, C₁₋₆alkyl-pyrrolidine-phenyl, C₁₋₆alkylC(O)-pyrrolidine, C₁₋₆alkylC(O)-pyrrolidine-phenyl, piperidine, piperidine phenyl, C₁₋₆alkyl-piperidine, piperidine-C₁₋₆alkyl-phenyl, C₁₋₆alkylC(O)-piperidine, C₁₋₆alkylC(O)-piperidine-phenyl.

In some embodiments, R or R¹ is further substituted with OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, and —C₀₋₆alkylNHC₀₋₆alkyl heterocyloalkyl, each of which is optionally substituted by one or more substituents selected from —C₀₋₆alkyl, —C₁₋₆alkylOC₁₋₆alkyl, halogen, or —C(O)OtButyl.

In some embodiments, the composition contains 1-10% compound B by weight.

In some embodiments, the composition contains 2-5% compound B by weight.

In some embodiments, R³ is F, Cl, Br or I.

In some embodiments, R³ is radioactive.

In some embodiments, the composition comprises the precursors used for radiolabeling and the radioactive tracers.

In some emblements, R is NH—R¹

In some embodiments, the present invention provides a composition comprising compound having the structure:

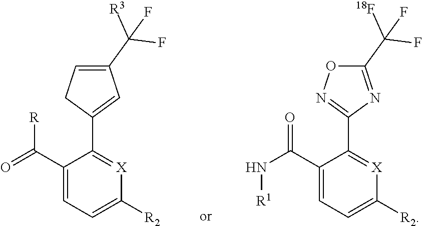

In some embodiments, the present invention provides a composition comprising compound having the structure:

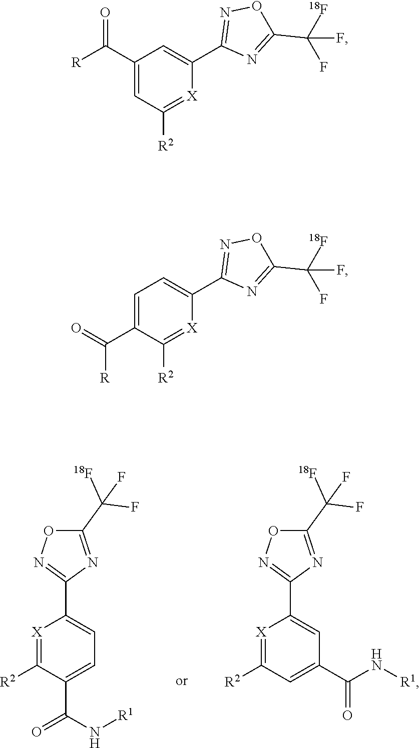

X is C or N;
R² is H or halogen; and
R³ is halogen
   R or R¹ is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
   each of which is optionally substituted by one or more substituents selected from —C₀₋₆alkyl, —C₁₋₆alkylOC₁₋₆alkyl, halogen, or —C(O)OtButyl.

In some embodiments, R is NH—R¹.

In some embodiments, R¹ is C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-heteroaryl-aryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl.

In some embodiments, R¹ is C₁₋₆alkylC(O)NC₁₋₆alkyl phenyl, C₁₋₆alkyl-N—C₁₋₆alkyl phenyl, C₁₋₆alkyl-N-oxazole-phenyl, C₁₋₆alkyl-oxazole-phenyl, C₁₋₆alkylC(O)—C₁₋₆alkyl-NH—C₁₋₆alkyl phenyl, pyrrolidine, pyrrolidine phenyl, C₁₋₆alkyl-pyrrolidine, C₁₋₆alkyl-pyrrolidine-phenyl, C₁₋₆alkylC(O)-pyrrolidine, C₁₋₆alkylC(O)-pyrrolidine-phenyl, piperidine, piperidine phenyl, C₁₋₆alkyl-piperidine, piperidine-C₁₋₆alkyl-phenyl, C₁₋₆alkylC(O)-piperidine, C₁₋₆alkylC(O)-piperidine-phenyl.

In some embodiments, R or R¹ is further substituted with OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, and —C₀₋₆alkylNHC₀₋₆alkyl heterocyloalkyl, each of which is optionally substituted by one or more substituents selected from —C₀₋₆alkyl, —C₁₋₆alkylOC₁₋₆alkyl, halogen, or —C(O)OtButyl.

In some embodiments, R or R¹ is H, OH, methyl, formyl, or carbonyl.

In some embodiments, R or R¹ is OH.

In some embodiments, R or R¹ is methyl.

In some embodiments, R or R¹ is formyl, or carbonyl.

In some embodiments, R or R¹ is —O—C₁₋₆ alkyl.

In some embodiments, R or R¹ is —O-methyl, —O-ethyl or —O-tert-butyl.

In some embodiments, R² is H, F, Cl, Br or I.

In some embodiments, R² is H.

In some embodiments, R² is F.

In some embodiments, X is C.

In some embodiments, X is N.

In some embodiments, R¹ is selected form the group consisting of:

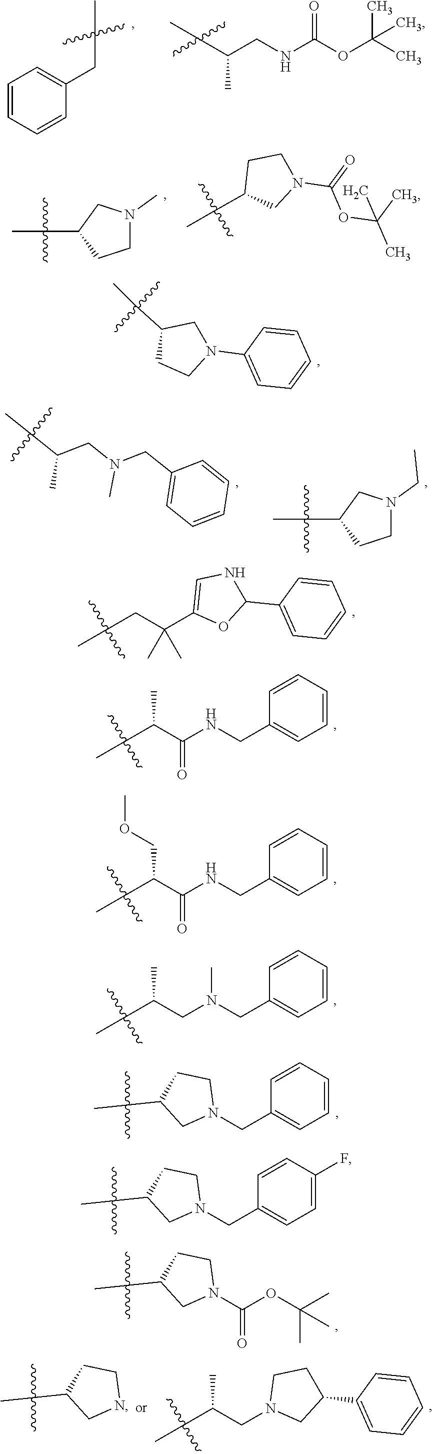

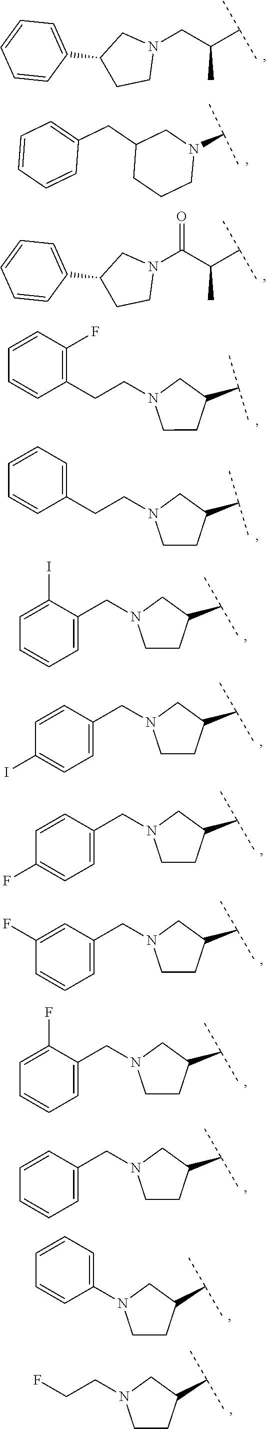

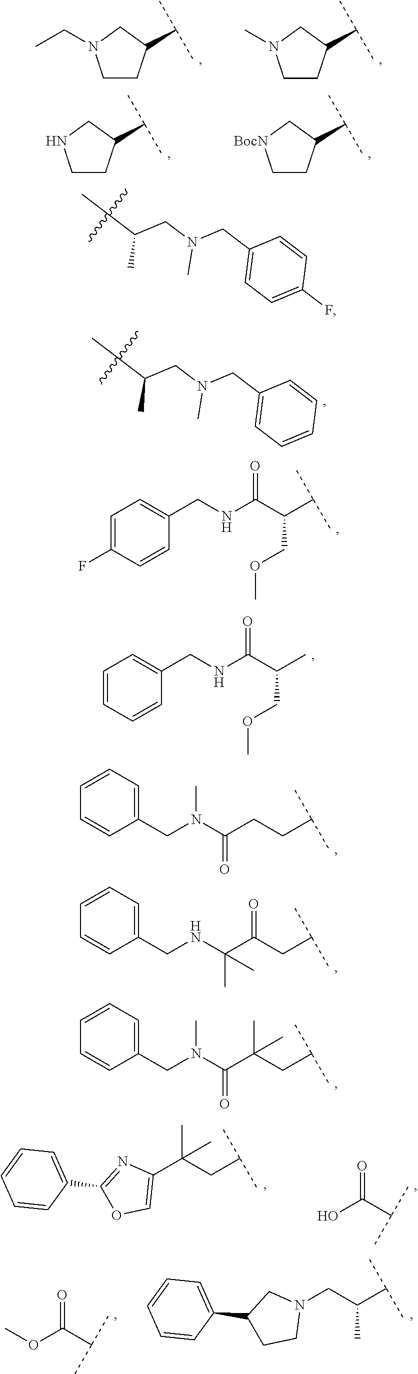

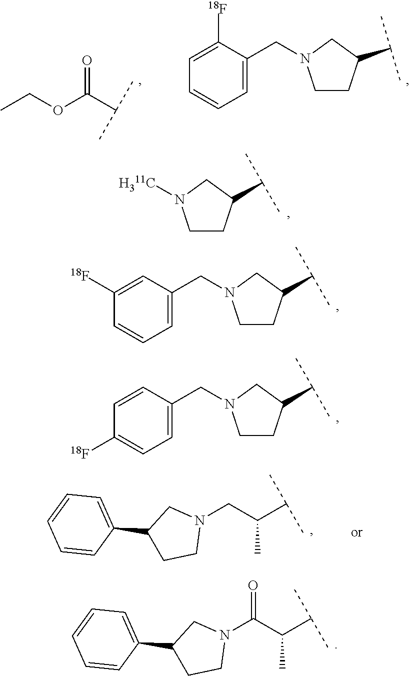

In some embodiments, the present invention provides a composition comprising compound having the structure:

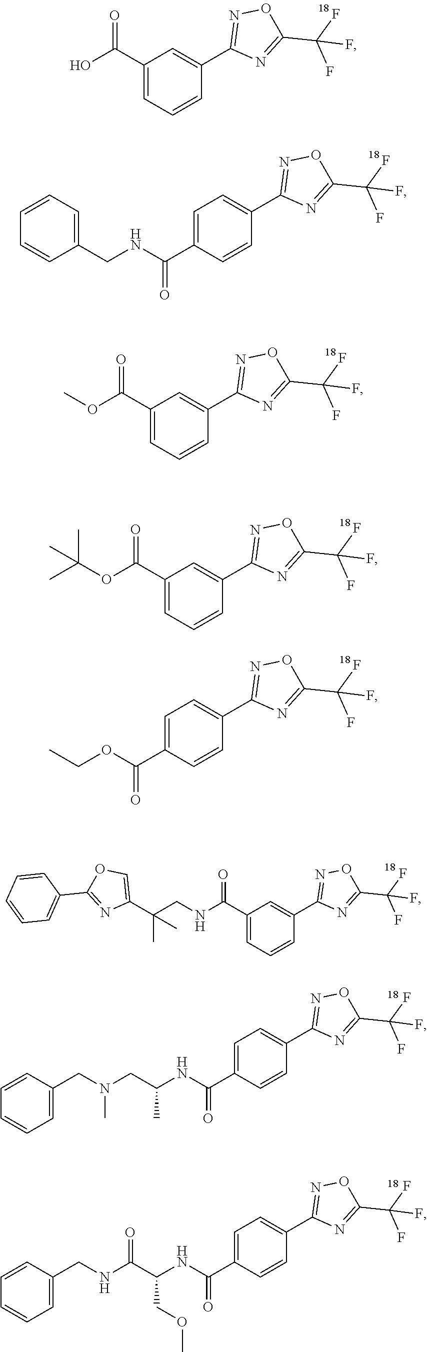

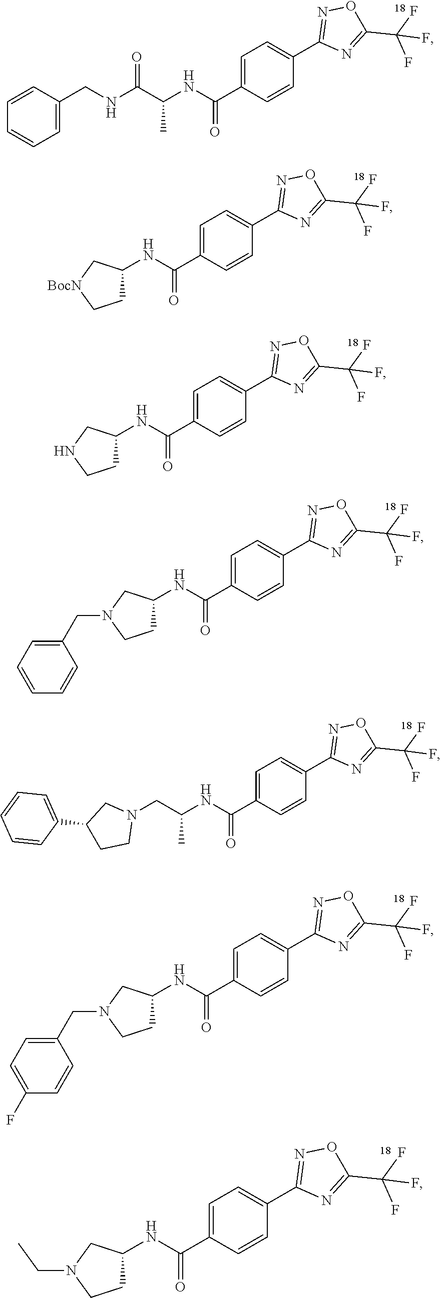

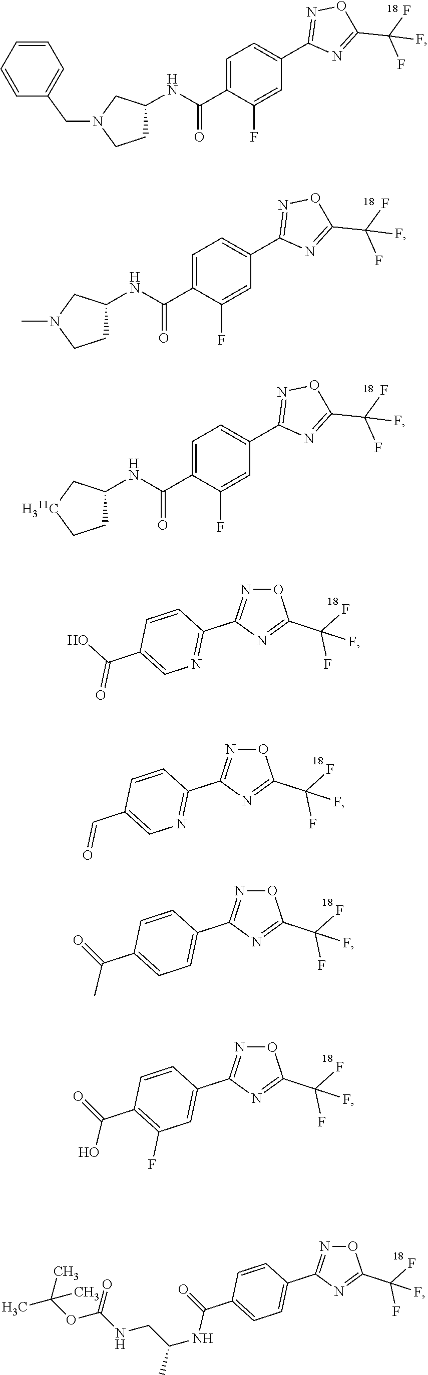

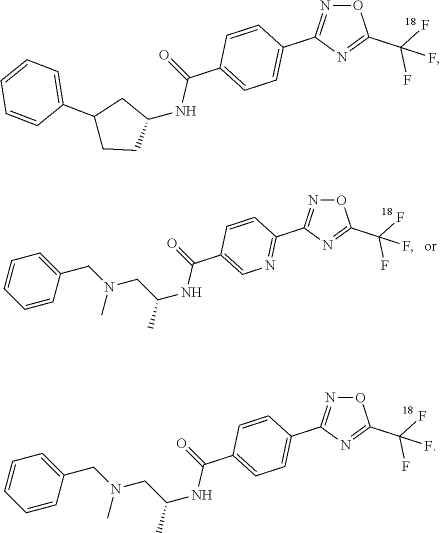

In some embodiments, the present invention provides a composition comprising human histone deacetylases (HDACs) inhibitors.

In some embodiments, the HIDACs are HDAC-4, HDAC-5, HDAC-7 and HDAC-9.

In some embodiments, the present invention provides a process for preparing compound IV(a) having the structure:
comprising:

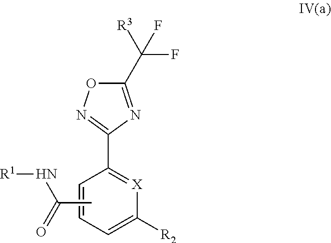

a) reacting compound I(a) having the structure:

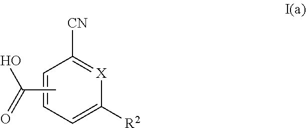

with hydroxylamine with water and ethanol to obtain compound II(a) having the structure:

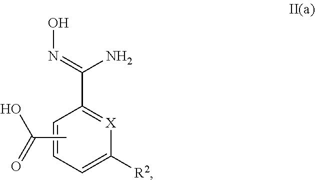

b) reacting compound II(a) with

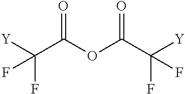

in pyridine to obtain compound IIIc having the structure:

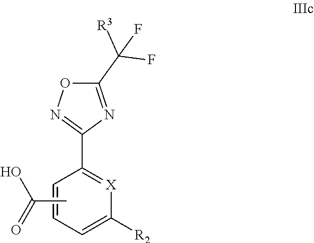

c) reacting compound IIIc with an amine in 1-[Bis(dimethylamino)methylene]-1H-1,2,3-triazolo[4,5-b]pyridinium 3-oxid hexafluorophosphate (HATU) and an excess amount of 4-methylmorpholine (NMM) to produce compound IVa having the structure:

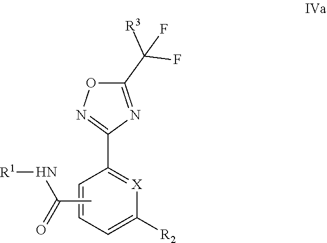

wherein:
X is C or N;
R¹ is selected from the group consisting of H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen;
R² is halogen; and
Y is halogen.

In some embodiments, the current invention provides a process for preparing compound III having the structure:

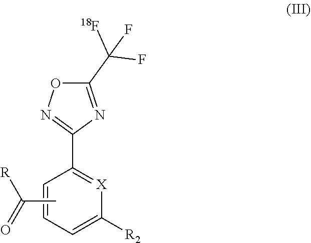

comprising
   a) reacting R—H with compound IIIa having the structure:

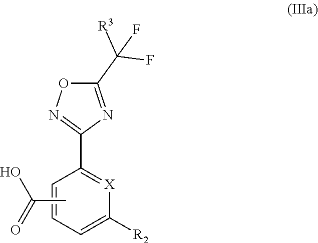

in 1-[Bis(dimethylamino)methylene]-1H-1,2,3-triazolo[4,5-b]pyridinium 3-oxid hexafluorophosphate (HATU), 4-methylmorpholine (NMM) and dimethylformamide (DMF) to obtain the compound IIIb having the structure:

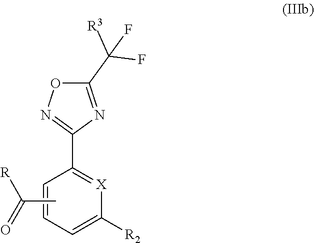

b) reacting compound IIIb with a [¹⁸F] fluorinating agent in a solvent to obtain compound III having structure:

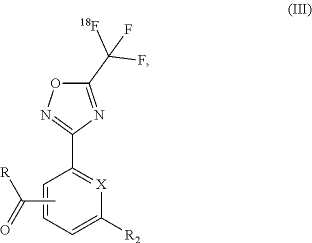

wherein:
X is C or N;
R is H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen;
wherein the aryl, heteroaryl, cycloalkyl, heterocyloalkyl is substituted or unsubstituted.

In some embodiments, R is C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-heteroaryl-aryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl.

In some embodiments, R is C₁₋₆alkylC(O)NC₁₋₆alkyl phenyl, C₁₋₆alkyl-N—C₁₋₆alkyl phenyl, C₁₋₆alkyl-N-oxazole-phenyl, C₁₋₆alkyl-oxazole-phenyl, C₁₋₆alkylC(O)—C₁₋₆alkyl-NH—C₁₋₆alkyl phenyl, pyrrolidine, pyrrolidine phenyl, C₁₋₆alkyl-pyrrolidine, C₁₋₆alkyl-pyrrolidine-phenyl, C₁₋₆alkylC(O)-pyrrolidine, C₁₋₆alkylC(O)-pyrrolidine-phenyl, piperidine, piperidine phenyl, C₁₋₆alkyl-piperidine, piperidine-C₁₋₆alkyl-phenyl, C₁₋₆alkylC(O)-piperidine, C₁₋₆alkylC(O)-piperidine-phenyl.

In some embodiments, R is further substituted with OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, and —C₀₋₆alkylNHC₀₋₆alkyl heterocyloalkyl, each of which is optionally substituted by one or more substituents selected from —C₀₋₆alkyl, —C₁₋₆alkylOC₁₋₆alkyl, halogen, or —C(O)OtButyl.

In some embodiments, the [¹⁸F] fluorinating agent in a solvent is Cs ¹⁸F and K₂₂₂ in N, N-dimethylacetamide (DMA) or dimethyl sulfoxide (DMSO).

In some embodiments, R³ is Cl, Br or I.

In some embodiments, compound III has the structure:

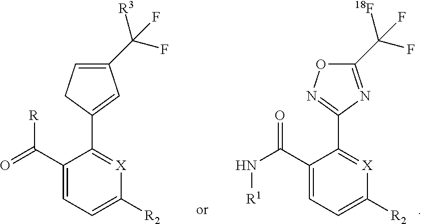

In some embodiments, compound III has the structure:

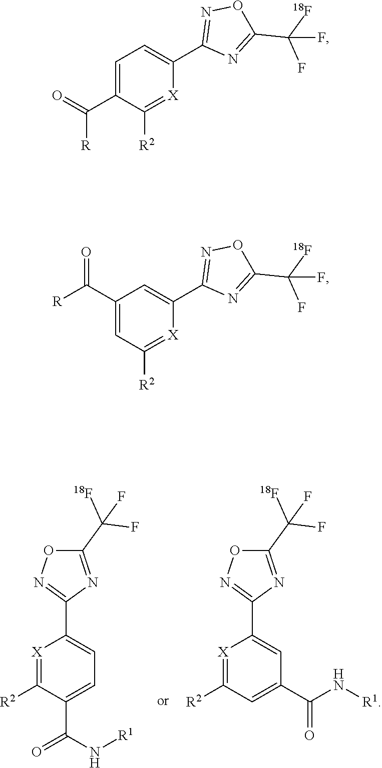

X is C or N;
R² is H or halogen;
R³ is halogen; and
R¹ is H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl.

In some embodiments, R or R¹ is H.

In some embodiments, R or R¹ is OH.

In some embodiments, R or R¹ is methyl.

In some embodiments, R or R¹ is formyl, or carbonyl.

In some embodiments, R or R¹ is —O—C₁₋₆ alkyl.

In some embodiments, R or R¹ is —O-methyl, —O-ethyl or —O-tert-butyl.

In some embodiments, R² is F, Cl, Br or I.

In some embodiments, R² is H.

In some embodiments, R² is F.

In some embodiments, X is C.

In some embodiments, X is N.

In some embodiments, R¹ is selected form the group consisting of:

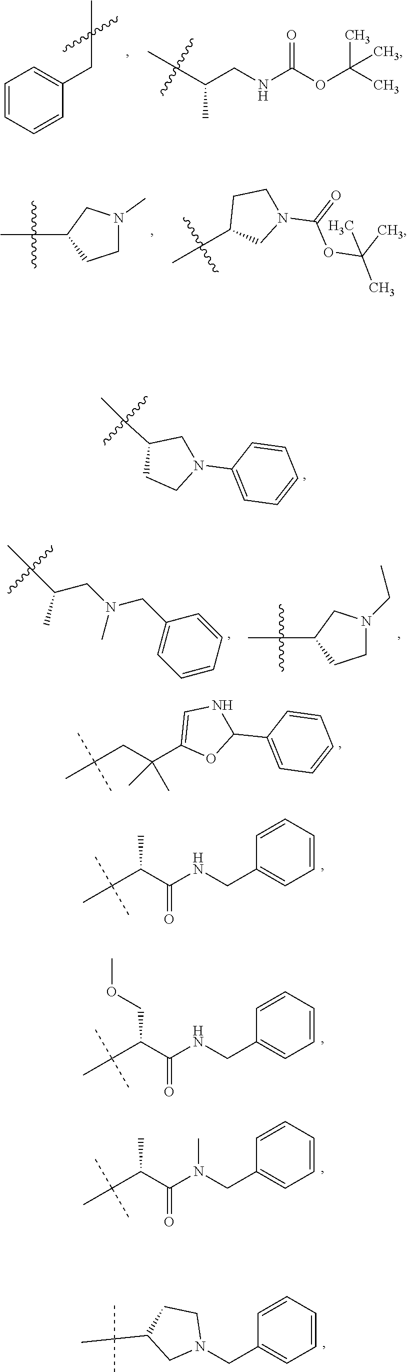

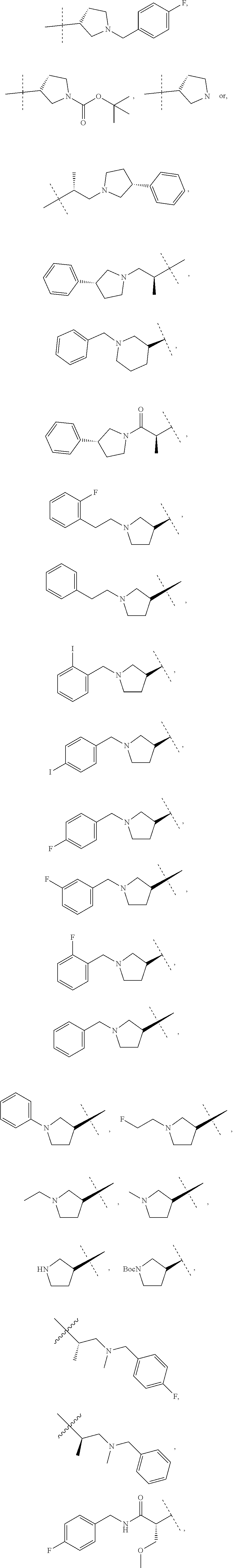

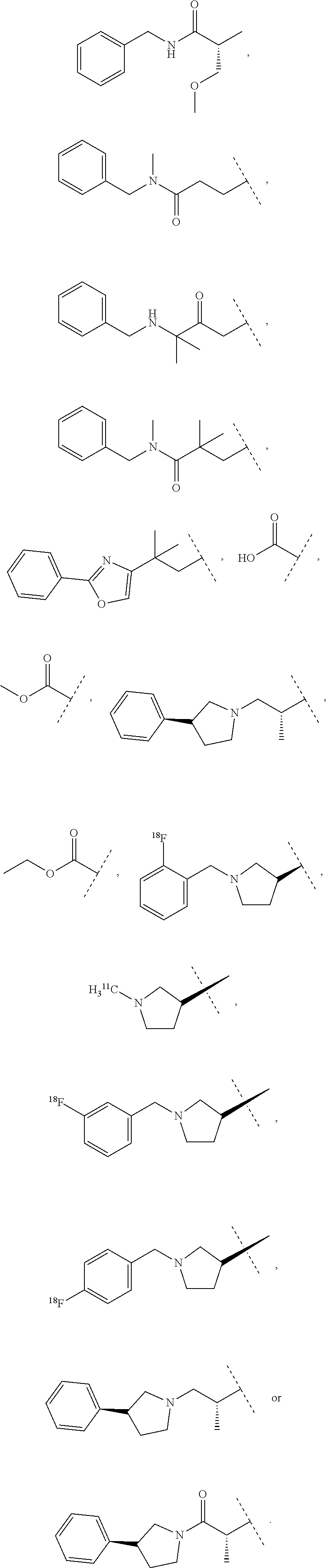

In some embodiments, compound III has the structure:

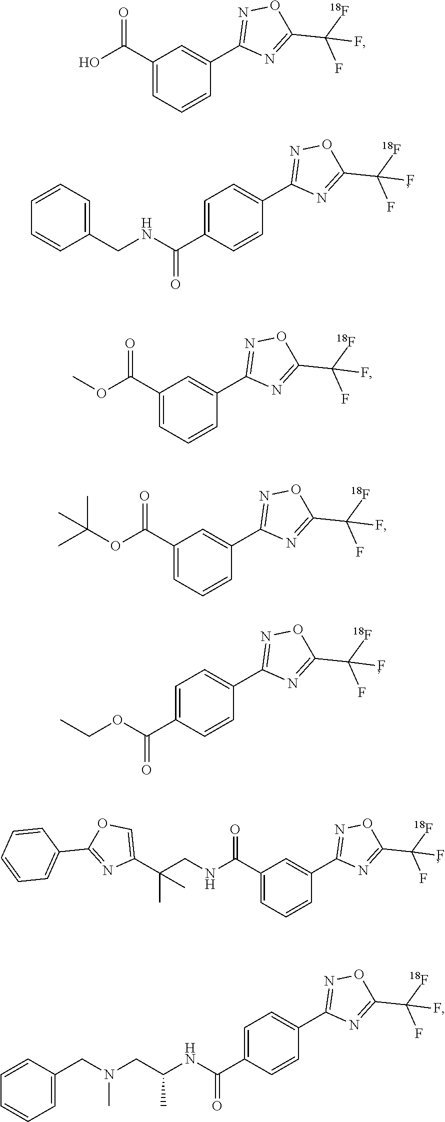

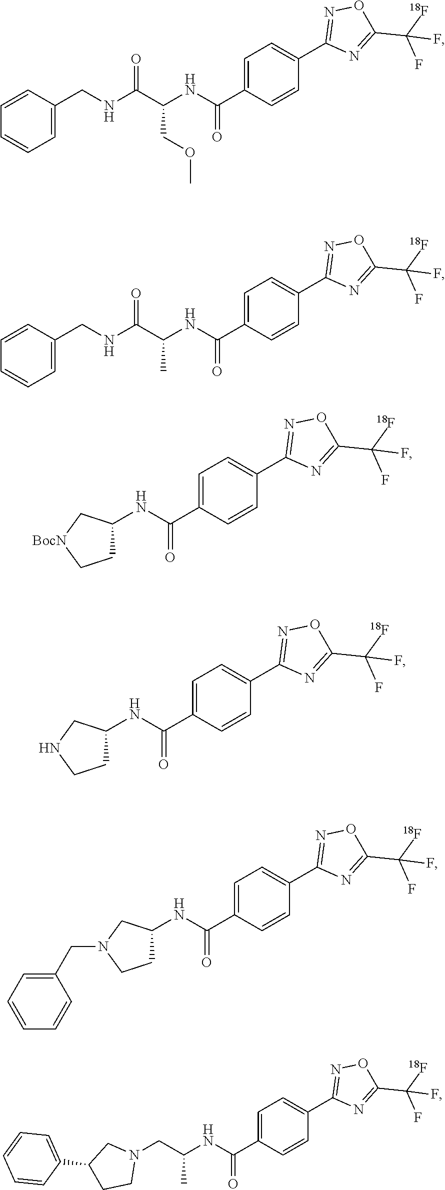 [SMILES: COC[C@@H](NC(=O)c1ccc(-c2noc(C(F)(F)[18F])n2)cc1)C(=O)NCc1ccccc1 ; C[C@@H](NC(=O)c1ccc(-c2noc(C(F)(F)[18F])n2)cc1)C(=O)NCc1ccccc1 ; CC(C)(C)OC(=O)N1CC[C@@H](NC(=O)c2ccc(-c3noc(C(F)(F)[18F])n3)cc2)C1 ; O=C(N[C@@H]1CCNC1)c1ccc(-c2noc(C(F)(F)[18F])n2)cc1 ; O=C(N[C@@H]1CCN(Cc2ccccc2)C1)c1ccc(-c2noc(C(F)(F)[18F])n2)cc1 ; C[C@H](CN1CC[C@H](c2ccccc2)C1)NC(=O)c1ccc(-c2noc(C(F)(F)[18F])n2)cc1]

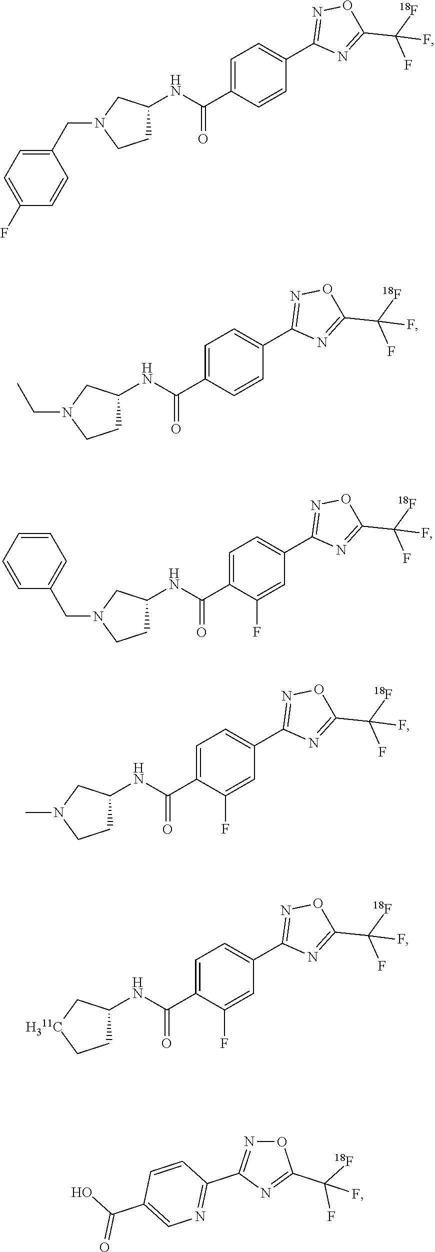 [SMILES: O=C(N[C@@H]1CCN(Cc2ccc(F)cc2)C1)c1ccc(-c2noc(C(F)(F)[18F])n2)cc1 ; CCN1CC[C@@H](NC(=O)c2ccc(-c3noc(C(F)(F)[18F])n3)cc2)C1 ; O=C(N[C@@H]1CCN(Cc2ccccc2)C1)c1ccc(-c2noc(C(F)(F)[18F])n2)cc1F ; CN1CC[C@@H](NC(=O)c2ccc(-c3noc(C(F)(F)[18F])n3)cc2F)C1 ; O=C(N[C@@H]1CC[11CH2]C1)c1ccc(-c2noc(C(F)(F)[18F])n2)cc1F ; O=C(O)c1ccc(-c2noc(C(F)(F)[18F])n2)nc1]

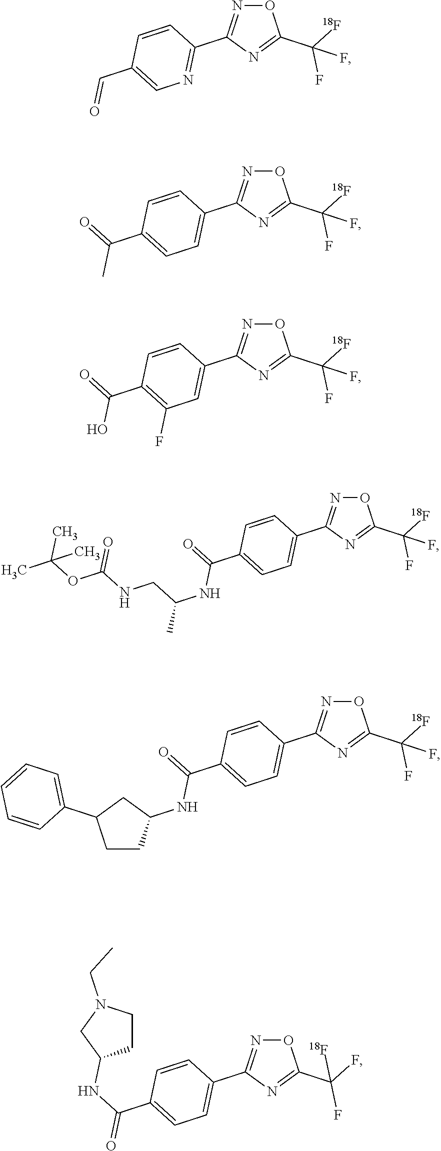

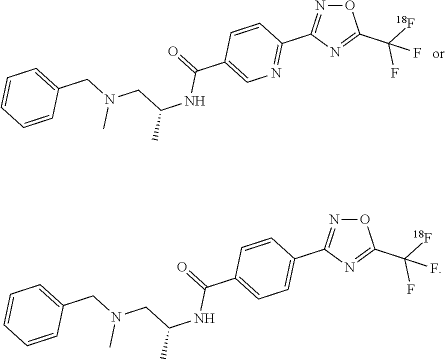 [SMILES: C[C@H](CN(C)Cc1ccccc1)NC(=O)c1ccc(-c2noc(C(F)(F)[18F])n2)nc1 ; C[C@H](CN(C)Cc1ccccc1)NC(=O)c1ccc(-c2noc(C(F)(F)[18F])n2)cc1]

In some embodiments, compound III is a human histone deacetylases (HDACs) inhibitor.

In some embodiments, HDACs are HDAC-4, HDAC-5, HDAC-7 and HDAC-9.

In some embodiments, the current invention provides a process for preparing compound IV having the structure:

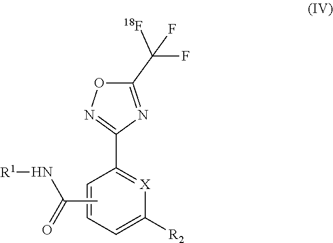

comprising:
   a) reacting compound I having the structure:

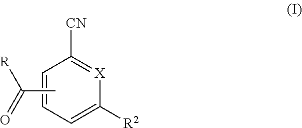

with hydroxylamine to obtain compound II having the structure:

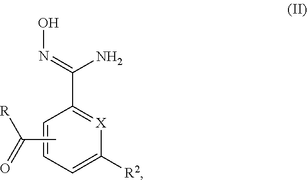

b) reacting compound II with bromodifluoroacetic anhydride in pyridine to produce compound IIIb having the structure:

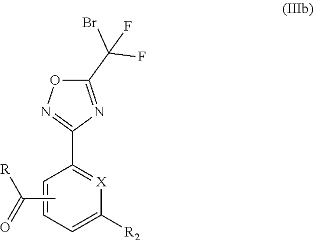

c) reacting compound IIIb with an amine in 1-[Bis(dimethylamino)methylene]-1H-1,2,3-triazolo[4,5-b]pyridinium 3-oxid hexafluorophosphate (HATU) and an excess amount of 4-methylmorpholine (NMM) to obtain compound IVb having the structure:

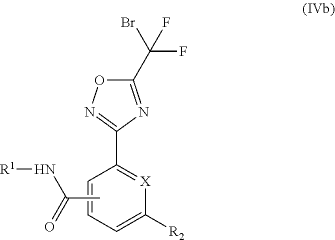

d) further reacting compound IVb with a [¹⁸F] fluorinating agent in a solvent to obtain compound IV having the structure:

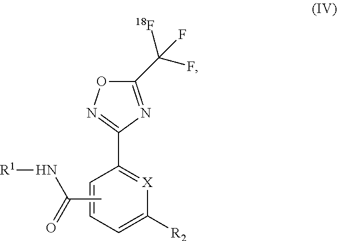

wherein:
X is C or N;
R is H, OH, O—C₁₋₆ alkyl, —C₁₋₆alkenyl, —C₁₋₆alkynyl, carbonyl, formyl or R¹;
R¹ is selected from the group consisting of —C₁₋₆alkyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl; each of which is optionally substituted by one or more substituents selected from —C₀₋₆alkyl, —C₁₋₆alkylOC₁₋₆alkyl, halogen, or —C(O)OtButyl; and
R² is H or halogen;
wherein the aryl, heteroaryl, cycloalkyl, heterocyloalkyl is substituted or unsubstituted.

In some embodiments, the [¹⁸F] fluorinating agent in a solvent is potassium ¹⁸F-fluoride (K¹⁸F) in dimethyl sulfoxide (DMSO).

In some embodiments, R or R¹ is H.

In some embodiments, R or R¹ is OH.

In some embodiments, R or R¹ is methyl.

In some embodiments, R or R¹ is formyl, or carbonyl.

In some embodiments, R or R¹ is —O—C₁₋₆ alkyl.

In some embodiments, R or R¹ is —O-methyl, —O-ethyl or —O-tert-butyl.

In some embodiments, R² is F, Cl, Br or I.

In some embodiments, R² is H.

In some embodiments, R² is F.

In some embodiments, X is C.

In some embodiments, X is N.

In some embodiments, [symbol] means a racemic mixture.

In some embodiments, compound IV has the structure:

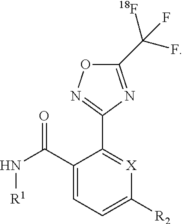

In some embodiments, compound IV has the structure:

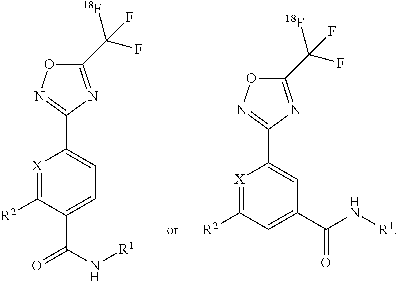

In some embodiments, R is selected form the group consisting of:

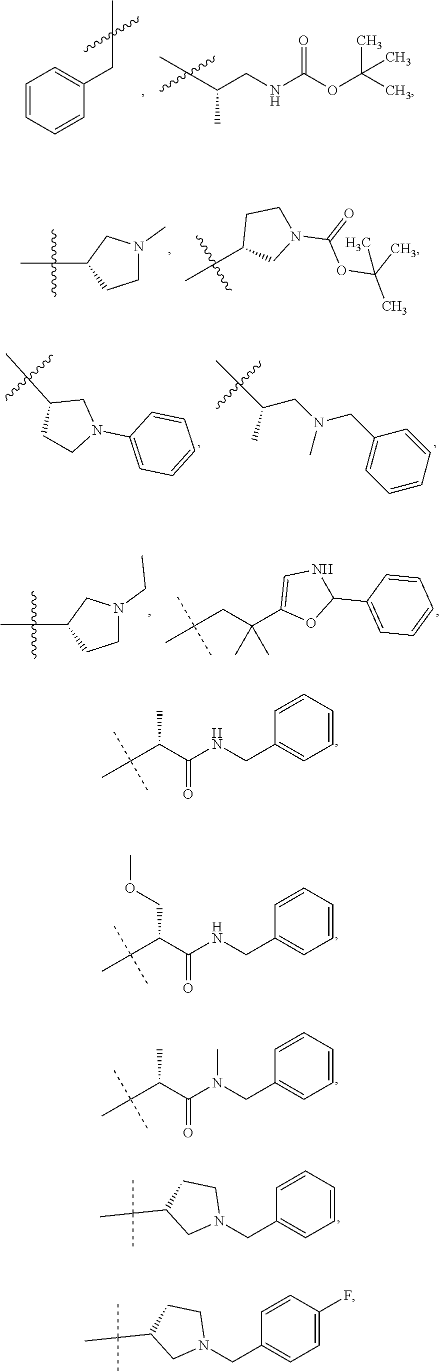

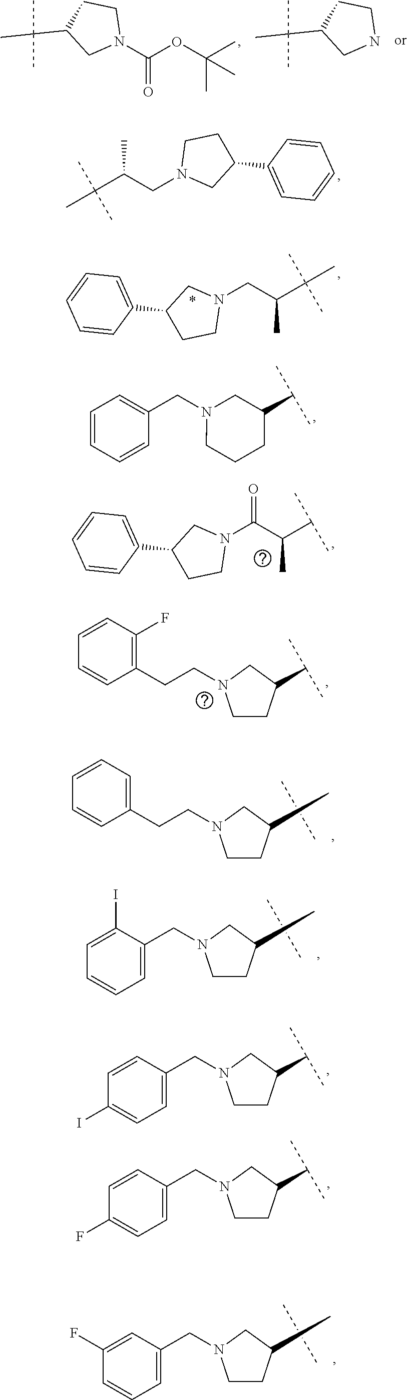

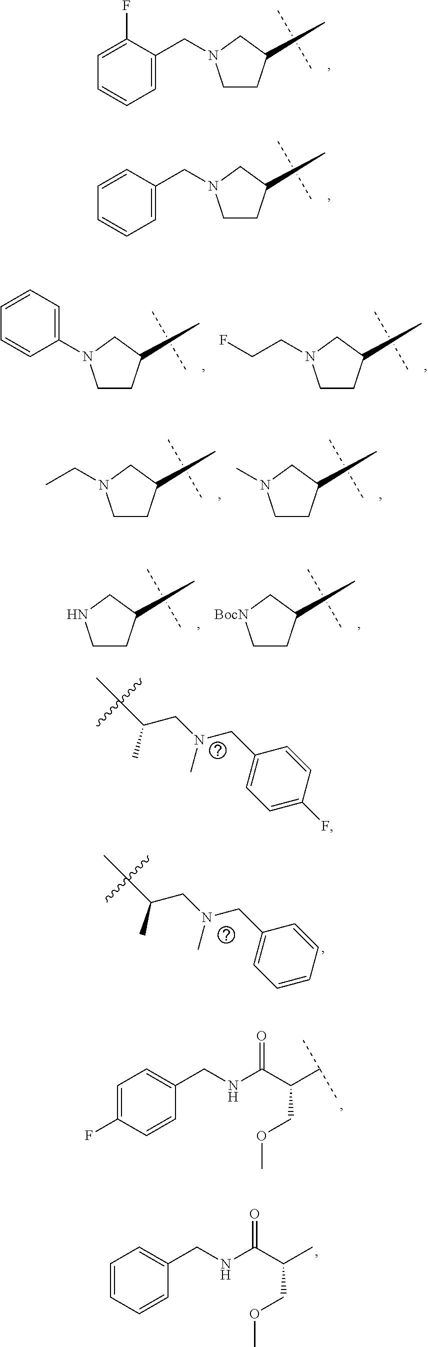

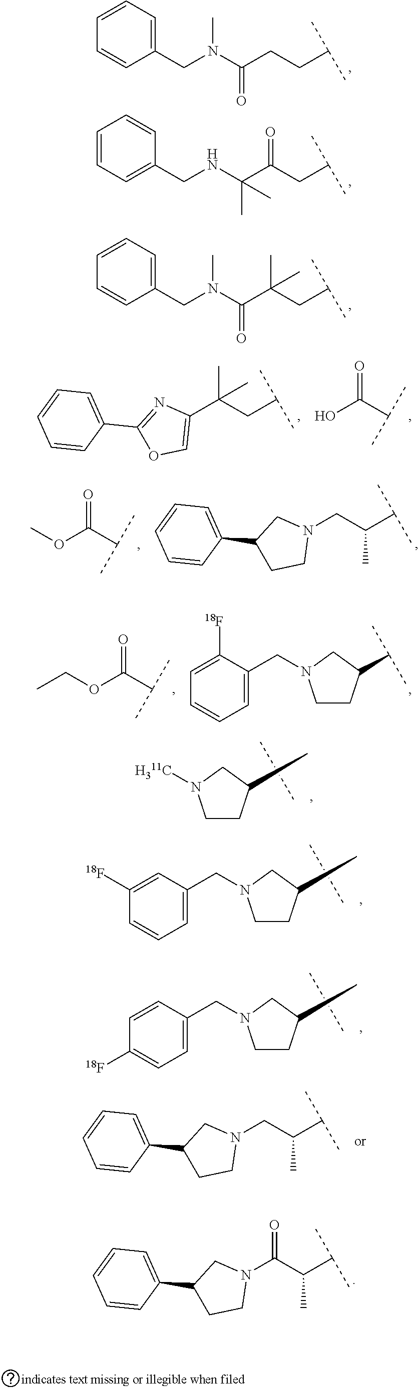

In some embodiments, compound IV has the structure:

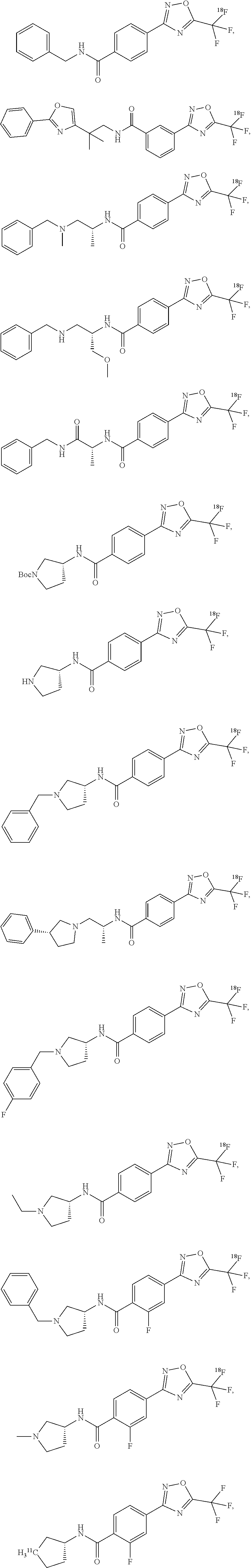

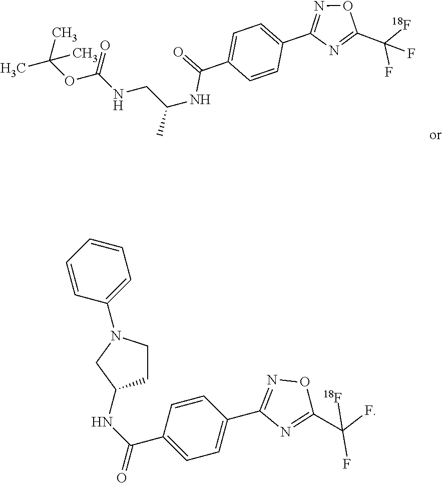

In some embodiments, compound IV is a human histone deacetylases (HDACs) inhibitor.

In some embodiments, HDACs are HDAC-4, HDAC-5, HDAC-7 and HDAC-9.

In some embodiments, the process for preparing compound IV is a late stage radiochemical synthesis,

In some embodiments, compound IIIb is reacted with Cs ¹⁸F and K₂₂₂ in DMSO to obtain compound II having the structure:

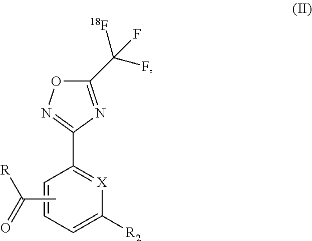

then compound II is further reacted with NaOH to obtain compound V having the structure:

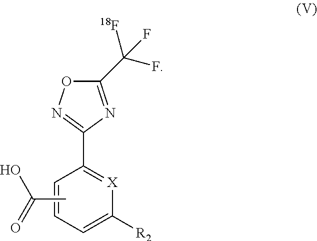

In some embodiments, compound IIIb is reacted with benzyl amine.

In some embodiments, the process for preparing compound V is a late stage radiochemical synthesis, In some embodiments, compound IIIb is

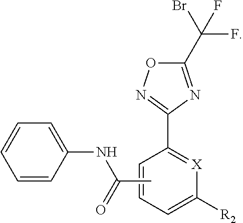

In some embodiments, wherein compound V is

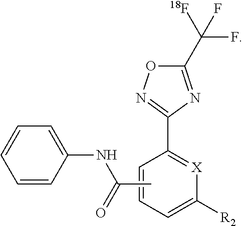

In some embodiments, compound IVb is

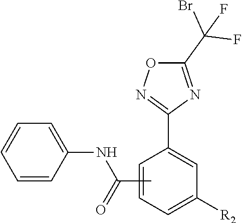

In some embodiments, compound IV is

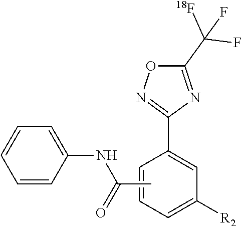

In some embodiments, the DMSO is added at a temperature range from 145° C. to 160° C.

In some embodiments, the DMSO is added at 150° C.

In some embodiments, the current invention provides a method of detecting a disease of the central nervous system (CNS) in a subject, the method comprising:
a) administering a radiolabeled histone deacetylase (HDAC) inhibitor in a mammal having the structure:

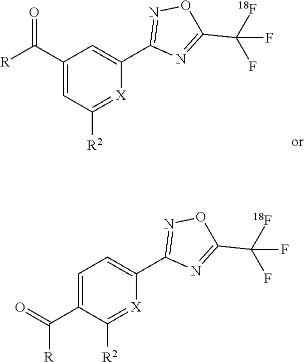

wherein:
X is CH or N;
   R is H, OH, —O—C₁₋₆ alkyl, carbonyl, C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl; and
R² is H or halogen;
b) detecting the presence of the radiolabeled HDAC inhibitor in the mammal using positron emission tomography (PET).

In some embodiments, R is H, OH, —O—C₁₋₆ alkyl, carbonyl, C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, or —C₀₋₆alkylNHC₀₋₆alkyl heterocyloalkyl.

In some embodiments, R is H.

In some embodiments, R is OH.

In some embodiments, R is methyl.

In some embodiments, R is formyl, or carbonyl.

In some embodiments, R is —O—C₁₋₆ alkyl.

In some embodiments, R is —O-methyl, —O-ethyl or —O-tert-butyl.

In some embodiments, R² is F, Cl, Br or I.

In some embodiments, R² is H.

In some embodiments, R² is F.

In some embodiments, X is C.

In some embodiments, X is N.

In some embodiments, ¹⁸F source used in radiofluorination reaction is Cs¹⁸F

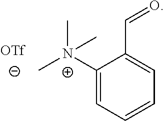

¹⁸F—F₂, or any molecule containing ¹⁸F which is commonly used in PET imaging.

In some embodiments, R¹ is selected form the group consisting of:

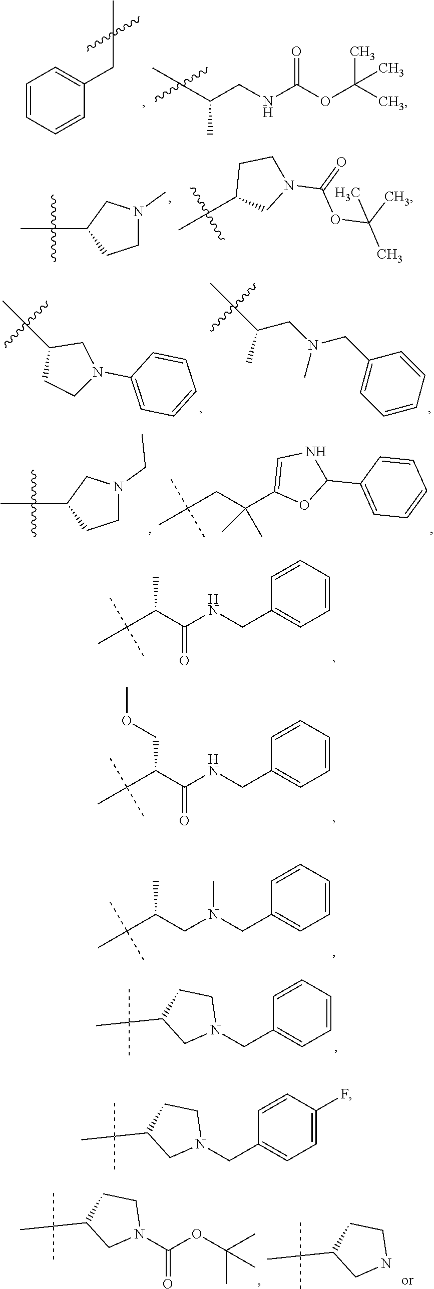

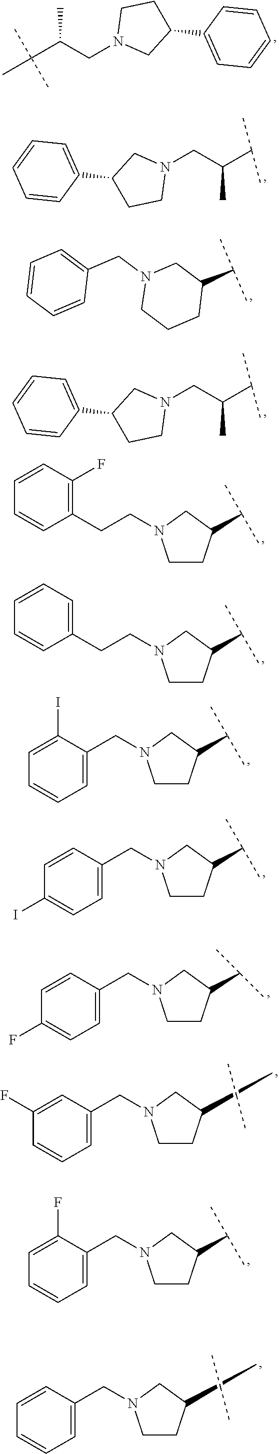

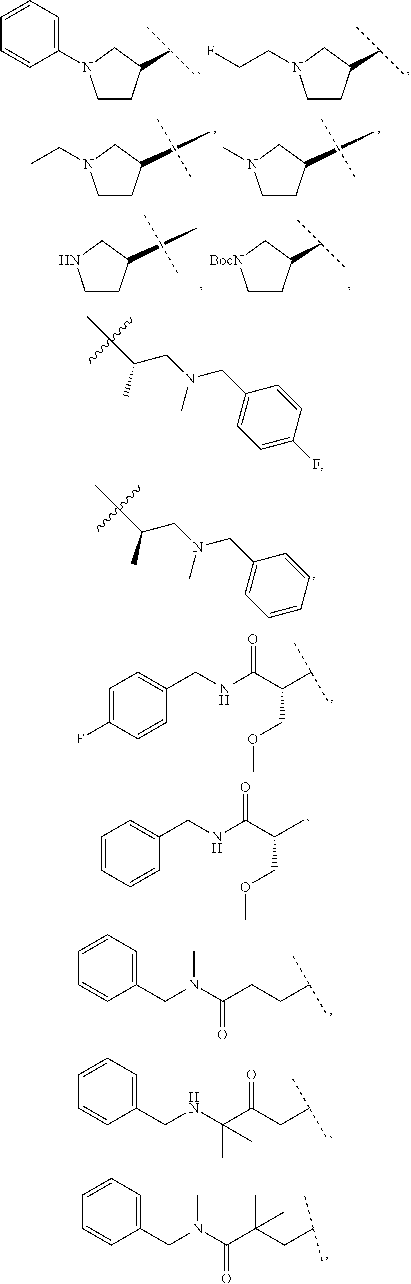

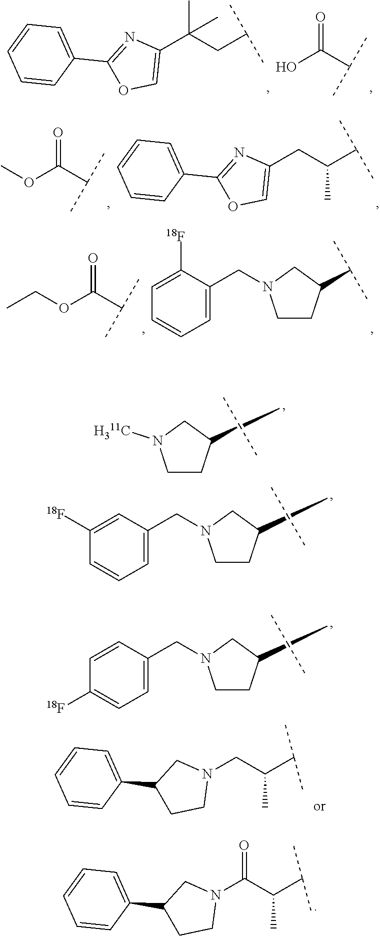

In some embodiments, HDACs inhibitor has the structure:

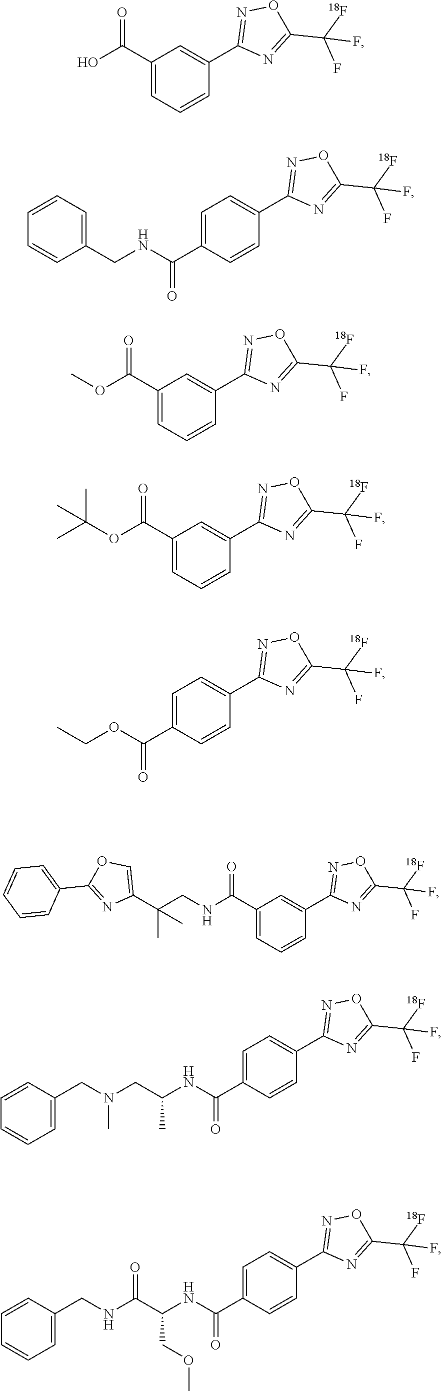

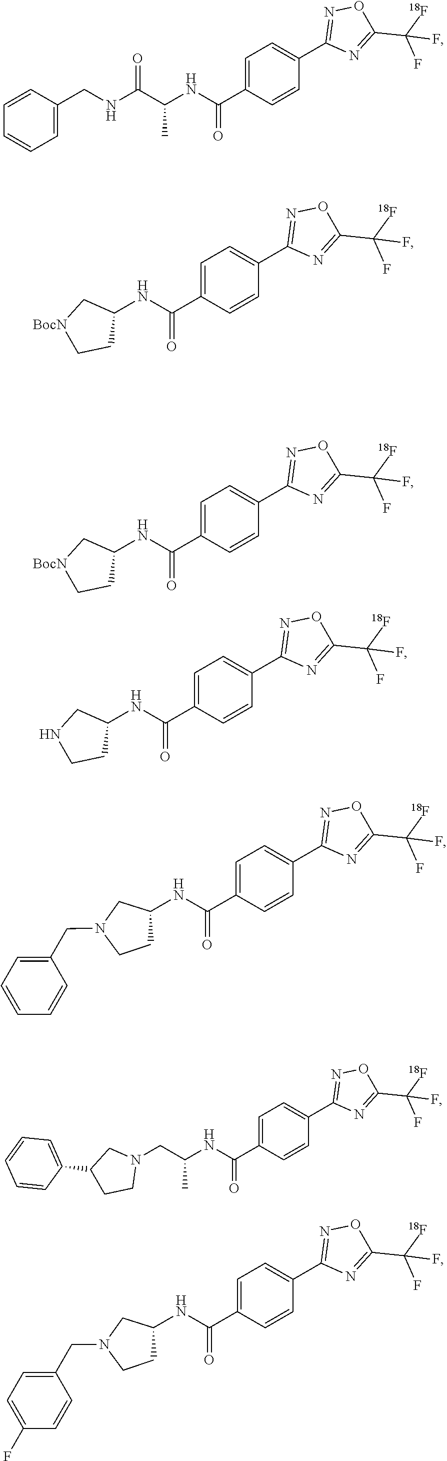

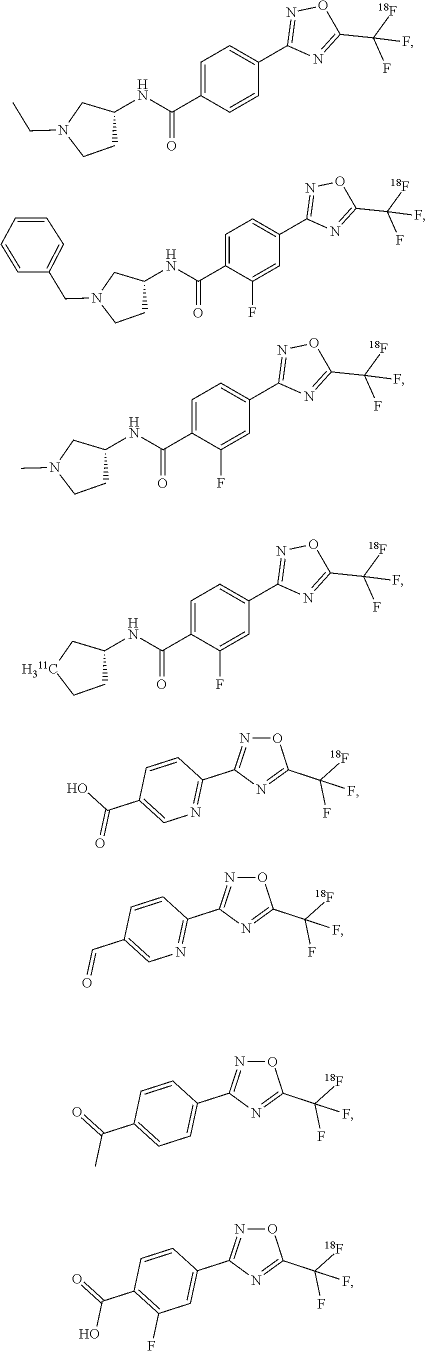

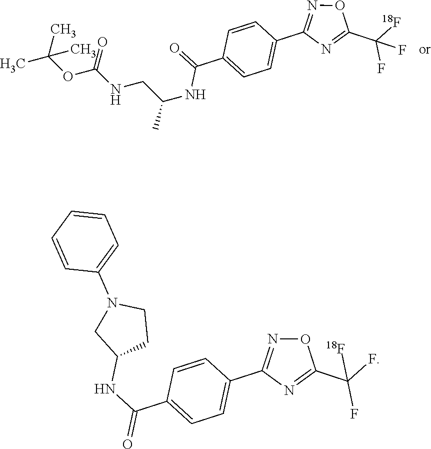 [SMILES: C[C@H](CNC(=O)OC(C)(C)C)NC(=O)c1ccc(-c2noc(C(F)(F)[18F])n2)cc1 ; O=C(N[C@H]1CCN(c2ccccc2)C1)c1ccc(-c2noc(C(F)(F)[18F])n2)cc1]

In some embodiments, the mammal is a rodent.

In some embodiments, the rodent is a rat.

In some embodiments, the disease is cancer.

In some embodiments, the HDAC inhibitors has blood-brain barrier (BBB) permeability.

In some embodiments, the HDAC inhibitors are used for brain imaging.

In some embodiments, the disease is cancer, disorders of the central nervous system, memory and cognitive impairment, dementia, rheumatoid arthritis or behavioral changes.

In some embodiments, the HDAC inhibitors accumulated in the liver of a mammal.

In some embodiments, the HDAC inhibitors accumulated in the brown fat adipose tissues (BATs).

In some embodiments, the present invention provides a method of identifying a suitable HDAC inhibitor which comprises creating the library of HDAC inhibitors containing a ¹⁸F radiolabeled group, using the HDAC inhibitors to PET image a subject and identifying biodistribution of DAC inhibitors in the subject.

As used herein, a “symptom” associated with a disease or disorder includes any clinical or laboratory manifestation associated with the disease or disorder and is not limited to what the subject can feel or observe.

As used herein, ¹¹C-source is carbon dioxide (¹¹CO₂), carbon monoxide (¹¹CO), ¹¹CH₄, ¹¹CH₃ or any molecule containing ¹¹C that is commonly used in the PET imaging process.

As used herein,

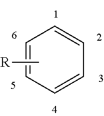

indicates the functional group R can be attached to either of 1, 2, 3, 4, 5 or 6 position.

As used herein,

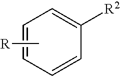

indicates the functional group R and R² can be in ortho, meta, or para position.

As used herein, “precursors” is a chemical compound preceding another in a metabolic pathway. The precursors used in the current invention were radiolabeled and produced compounds in Table 3.

As used herein, “radioactive tracer” is a chemical compound in which one or more atoms have been replaced by a radionuclide so by virtue of its radioactive decay.

As used herein, “treating”, e.g. of an infection, encompasses inducing prevention, inhibition, regression, or stasis of the disease or a symptom or condition associated with the infection.

As used herein, “radiochemical purity” or “RCP” is defined as the ratio of the activity of a radionuclide in a stated chemical species in a material over the total activity of all species containing that radionuclide in this material.

As used herein; “Biodistribution” is defined as a method of tracking where compounds of interest travel in an experimental animal or human subject.

As used herein, “about” mean±5%.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As used herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons and five hydrogens.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

In choosing the compounds of the present invention, one of ordinary skill in the art will recognize that the various substituents, i.e. R¹, R², etc. are to be chosen in conformity with well-known principles of chemical structure connectivity.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure result.

In choosing the compounds used in the method of the present invention, one of ordinary skill in the art will recognize that the various substituents, i.e. R¹, R², etc. are to be chosen in conformity with well-known principles of chemical structure connectivity.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

In choosing the compounds of the present invention, one of ordinary skill in the art will recognize that the various substituents, i.e. R¹, R², etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

While the invention has been shown and described with reference to certain embodiments of the present invention thereof, it will be understood by those skilled in the art that various changes in from and details may be made therein without departing from the spirit and scope of the present invention and equivalents thereof.

### EXPERIMENTAL DETAILS

### Example 1. Synthesis of Bromodifluorooxadiazoles and Intermediate Compounds

### Materials and Methods

Solvents and starting material were obtained from commercial sources and were used as received. High-Performance Liquid Chromatography (HPLC) was performed with a 1260 series pump, (Agilent Technologies, Stuttgart, Germany), with a built-in UV detector operated at 254 nm and a radioactivity detector with a single-channel analyzer (lab logic) using a semipreparative C18 reverse-phase column (10×250 mm, Phenomenex) and an analytical C18 column (4.6×250 mm, ASCENTIS RP-AMIDE, Sigma). An acetonitrile/ammonium acetate buffer (MeCN/NH₄QAc: 20 mM)) or acetonitrile/water (MeCN/water) solvent systems with various composition (solvent systems were developed specific to each compound), was used for quality control analyses at a flow of 1 mL/min. High resolution mass spectroscopy (HRMS) was performed using Agilent 1260HPLC/G6224A-TOF MS. NMR spectroscopy was performed using 400 MHz Bruker instrument.

### Chemical Synthesis

TMP195 was prepared similar to the previously described procedures (Lobera, M., et al., 2013)

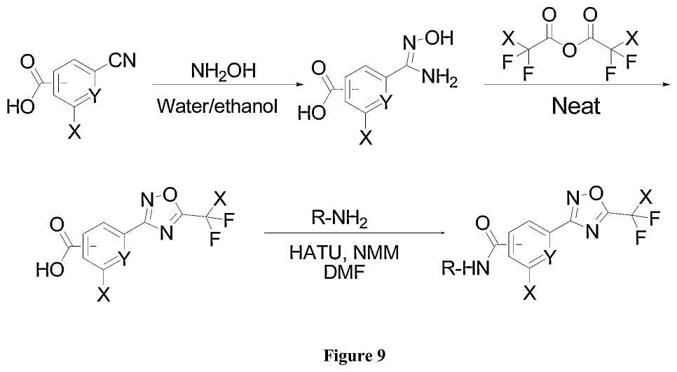

General procedure for amine/carboxylic acid coupling reactions (3-4, 12-13 and 23). The acid (1.0 eq) and HATU (1.2 eq.) in DMF (1.0-3.0 mL) were stirred for 15 min followed by simultaneous addition of the amine (1.2 eq) and NMM (excess: ˜1.0 mL). The reaction mixture was stirred for 3 hours. The DMF was removed under vacuum (˜70° C. water bath) and the residue was purified by column chromatography followed by trituration in cold pentane to afford the final products in 50-70% yield. The general procedure is described in Scheme A (FIG. 9)

General procedure for synthesis of (N′-Hydroxycarbamimidoyl) benzoic acid (7-8 and 16-17). To the nitrile (1.0 g) in 30 ml ethanol was added first hydroxylamine hydrochloric acid (1.0 g) dissolved in water (8.0 ml) followed by sodium carbonate (1.2 g) dissolve in water (12.0 ml). The mixture was heated under reflux for 4 h. Ethanol was removed under reduced pressure and the residue was diluted with water, acidified with 10% HCl to pH ˜3, and filtrated, washed with water and then dried under reduced pressure to afford compound (N′-hydroxycarbamimidoyl) benzoic acid in 50-80% yield.

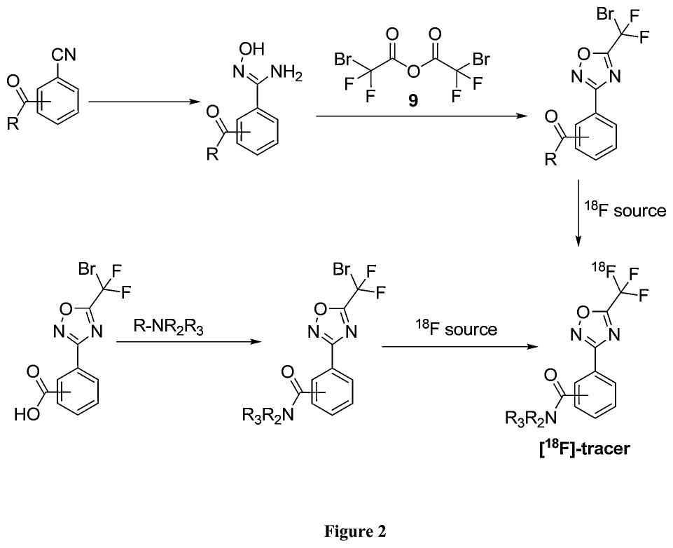

General synthesis of the bromodifluorooxadiazoles (bromo-Precursors: 10-11 and 20-21). Bromodifluoroacetic anhydride (2) ml neat was added to N′-hydroxycarbamimidoyl benzoic acid (1 g). The reaction mixture heated to 50° C. for 3 h. The volatiles were evaporated, and the crude product was purified by column chromatography using ethyl acetate/hexanes or dichloromethane/methanol to afford the bromodifluoro-analogs. The general scheme is shown in FIG. 2.

General synthesis of the trifluorooxadiazoles (19-20). The synthesis of the TFMO moiety was performed similar to the synthesis described above for bromodifluorooxadiazole except that neat trifluoroacetic anhydride was used.

### Characterization Data for the New Compounds

N-benzyl-3-(5-(trifluoromethyl)-1,2,4-oxadiazol-3-yl) benzamide (3): H NMR (400 MHZ, CDCb) o 8.80 (s, 1H), 8.30 (d, J=8.6 Hz, 1H), 8.08 (d, J=8.4 Hz, 1H), 7.64 (t, J=8.6 Hz, 1H), 7.34 (m, 5H), 6.56 (s, 1H), 4.70 (d, J=8.6 Hz, 2H). 13C NMR (126 MHZ): o 170.00 (t, J=23.9), 168.42, 166.10, 137.82, 135.48, 131.16, 130.54, 129.69, 128.89, 128.07, 127.82, 125.78, 125.56, 107.52 (t, J=217.60), 44.36. 19F NMR (CDCb), o −51.98. HRMS: Calculated for C11H13F3N302 19F NMR (CDCb), o −64.73. HRMS: Calculated for [M+H] 348.0954, Found: 348.0950. C11H13F3N302 [M+H] 348.0954, Found: 348.0950.

N-benzyl-4-(5-(trifluoromethyl)-1,2,4-oxadiazol-3-yl) benzamide (4): 1H NMR (CDCl3): o 8.20 (d, 2H), 7.95 (d, 2H), 7.35 (m, 5H), 6.5 (s, 1H), 4.69 (d, 2H). 13C NMR (126 MHZ): o 168.46, 166.25, 166.0 (q, J=31.8 Hz), 137.78, 137.75, 128.90, 128.03, 127.99, 127.84, 127.83, 127.69, 116.5 {q, J=196.2 Hz), 44.35. 19F NMR (CDCb), o −65.30. HRMS: Calculated for C11H13F3N302 [M+H] 348.0954, Found: 348.0952.

3-(5-(bromodifluoromethyl)-1,2,4-oxadiazol-3-yl) benzoic acid (10): 1H NMR (400 MHZ, DMSO-d6): o 8.54 (s, 1H), 8.27 (d, J=6, 1H), 8.19 (d, J=6, 1H), 7.75 (s, 1H). 13C NMR (126 MHZ) δ 170 (t, J=24), 168.28, 166.77, 133.45, 132.43, 131.79, 130.57, 128.40, 125.45, 107.52 (t, J=215.5). 19F NMR (CDCl3), o−54.01.

4-(5-(bromodifluoromethyl)-1,2,4-oxadiazol-3-yl) benzoic acid (11): 1H NMR (400 MHZ, DMSO-d6): o 8.14 (m, SH). 13C NMR (126 MHZ): 0170 (t, J=23.8), 168.28, 166.94, 134.60, 130.76, 128.71, 128.12, 107.52 (t, J=215.4). 19F NMR (CDCb), o−54.01.

N-benzyl-3-{5-(bromodifluoromethyl)-1,2,4-oxadiazol-3-yl)benzamide (12): 1H NMR (400 MHz, CDCb) o 8.49 (s, 1H), 8.28 (d, J=8.6 Hz, 1H), 8.08 (d, J=8.4 Hz, 1H), 7.64 (t, J=8.6 Hz, 1H), 7.34 (m, 5H), 6.56 (s, 1H), 4.70 (d, J=8.6 Hz, 2H). 13C NMR (126 MHZ): o 170.00 (t, J=23.9), 168.42, 166.10, 137.82, 135.48, 131.16, 130.54, 129.69, 128.89, 128.07, 127.82, 125.78, 125.56, 107.52 (t, J=217.60), 44.36. 19F NMR (CDCb), o −51.98. HRMS: Calculated for C11H138rF2N302 [M+H]: 408.0154, Found: 408.0152.

N-benzyl-4-(5-(bromodifluoromethyl)-1,2,4-oxadiazol-3-yl) benzamide (13): H NMR (400 MHZ, CDCb) o 8.20 (d, J=8.6 Hz, 2H}, 7.95 (d, J=8.4 Hz, 2H), 7.35 (m, 5H), 6.5 (s, 1H), 4.69 (d, J=5.6 Hz, 2H). 13C NMR (126 MHZ): o 170.01 (t, J=23.9), 168.33, 166.25, 137.78, 137.65, 128.91, 128.05, 127.99, 127.92, 127.86, 127.77, 107.07 (t, J=217.74), 44.37. 19F NMR (CDCb), −δ −51.97. HRMS: Calculated for C11H138rF2N302[M+H]: 408.0154, Found: 408.0153.

Ethyl 3-(5-(trifluoromethyl)-1,2,4-oxadiazol-3-yl)benzoate (17): 1H NMR (400 MHZ, CDCb) o 8.79 (s, 1H}, 8.31 (d, J=5.9 Hz, 1H), 8.28 (d, J=5.9 Hz, 1H}, 7.64 (t, J=S.9 Hz, 1H), 4.45 (q, J=5.37, J=10.7 Hz, 2H), 1.45 (t, J=5.34, 3,H). 13C NMR (126 MHZ): 5168.60, 166.98 (q, J=31, J=63 Hz}, 165.50, 165.49, 133.18, 131.83, 131.72, 129.33, 128.78, 125.32, 115.51 (q, J=194, J=388 Hz), 61.52, 14.31. 19F NMR (400 MHZ, CDCl3): o 65.34. HRMS: Calculated for C12H9F3N203 [M+H]: 287.0644, Found: 287.0638.

Ethyl 3-(5-(bromodifluoromethyl)-1,2,4-oxadiazol-3-yl)benzoate (18): 1H NMR (400 MHZ, CDCb) o 8.79 (s, 1H), 8.32 (d, J=5.9 Hz, 1H), 8.30 (d, J=5.9 Hz, 1H), 7.63 (t, J=5.9 Hz, 1H), 4.45 (q, J=5.37, J=10.7 Hz, 2H), 1.45 (t, J=5.34, 3H). 13C NMR (126 MHZ): 0169.98 (t, J=56.6 Hz), 168.50, 165.56, 133.11, 131.71, 131.68, 129.31, 128.79, 125.53, 107.11 (t, J=215 Hz), 61.52, 14.35. 19F NMR (400 MHZ, CDCl3): o 51.95. HRMS: Calculated for C12H10BrF2N203 [M+H]: 346.9843, Found: 346.9837.

3-(5-(bromodifluoromethyl)-1,2,4-oxadiazol-3-yl)-N-(2-methyl-2-(2-phenyloxazol-4-yl) propyl)benzamide (23): 1H NMR (400 MHZ, CDCb) o 8.66 (s, 1H) 8.2 (m, 5H}, 7.65 (t, J=6.6 Hz, 1H), 7.52 (s, 1H), 7.45 (m, 3H), 3.66 (d, J=4.0 Hz, 2H), 1.50 (s, 6H). 13C NMR (126 MHz): 0169.92 (t, J=33.4 Hz), 168.62, 166.15, 161.81, 149.14, 136.18, 133.09, 131.16, 131.09, 130.69, 130.45, 129.50, 127 0.09, 126.49, 125.84, 125.57, 107.13 (t, J=300 Hz), 50.62, 34.33, 25.37.19F NMR (400 MHz, CDCl3): o 51.88. HRMS: Calculated for C23H20BrF2N4Q3 [M+H]: 517.0681, Found: 517.0681.

### Example 2. Biochemical Evaluation of Tracer Candidates

Biochemical assay: Tracer candidate were screened against recombinant human HDACs 1-11 using 9 dilutions using the commercially available HDACs enzyme assay kits (BPS Bioscience, Inc.) as described in the manufacturer protocol. The IC₅₀ values are shown in Table. 1. The class lla HDAC fluorogenic assay functions in a two-step process: first, the class lla HDAC removes the trifluoroacetylate (TFA) from TFA-lysine substrate to generate free lysine which is recognized by trypsin and releases a fluorescent fluorophore (intact substrate is not recognized by trypsin) that can be detected using microplate reader (Lobera M 2013). IC₅₀ was determined using nonlinear fit curves (GraphPad Prism) (% HDAC activity inhibition vs. compound concentration).

Cell-based deacetylase assay. The cell-based assay was conducted similar to the previous work (Lobera M 2013; Luckhurst C A 2019) Briefly, compounds were diluted in 5 uL DMSO (11-point concentration curve, 5% final DMSO) and were added to 96 wells with positive (5 μM Trichostatin A (TSA)) and negative control (5 uL DMSO) wells. HT-29 cells (obtained from ATCC) with the addition of either 100 μM Boc-Lys-TFA (class-IIa selective substrate) or 200 μM Boc-Lys-Ac (class-I/IIb selective substrate) were plated into 96 well plates at 200,000 cells/well in 45 uL cellular assay buffer (RPMI without phenol red, 0.1% Fetal Bovine Serum) and were incubated for 3 h at 37° C. The deacetylation achieved by the addition of 50 uL HDAC developer solution (2.5 mg/ml trypsin in DMEM without Fetal Bovine Serum and 10% tween 80) for a further 1 h to sensitize the substrate and to lyse the cells. Fluorescent counts were read with microplate reader at an excitation wavelength of 360 nm and detection of emitted light of 460 nm.

TABLE 1
Class-lla HDAC inhibitors and IC₅₀ values for HDACs inhibition in HT-29 cells using class-
distinguishing fluorogenic substrates whole-cell assay.

| | | | HT-29 (IC50) | |
|---|---|---|---|---|
| | Compound | | Class-lla | Class-I |
| # | ID | Structure | (uM) | (uM) |
| 1 | NT3-118 | 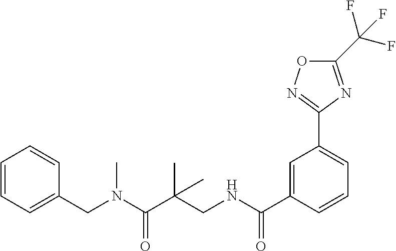 [SMILES: CN(Cc1ccccc1)C(=O)C(C)(C)CNC(=O)c1cccc(-c2noc(C(F)(F)F)n2)c1] | >50 | >50 |
| 2 | NT-175 | 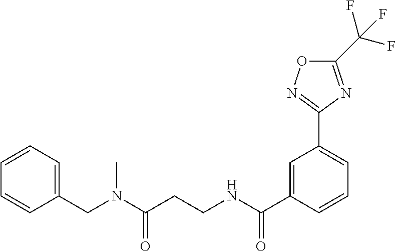 [SMILES: CN(Cc1ccccc1)C(=O)CCNC(=O)c1cccc(-c2noc(C(F)(F)F)n2)c1] | >50 | >50 |
| 3 | NT-157 | 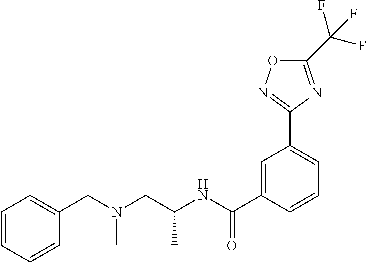 [SMILES: C[C@H](CN(C)Cc1ccccc1)NC(=O)c1cccc(-c2noc(C(F)(F)F)n2)c1] | >50 | >50 |
| 4 | NT-134 | 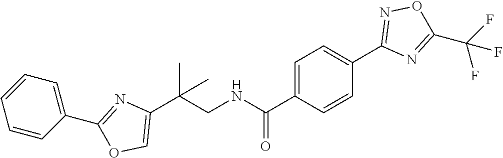 [SMILES: CC(C)(CNC(=O)c1ccc(-c2noc(C(F)(F)F)n2)cc1)c1coc(-c2ccccc2)n1] | >5 | >15 |
| 5 | NT3-119 | 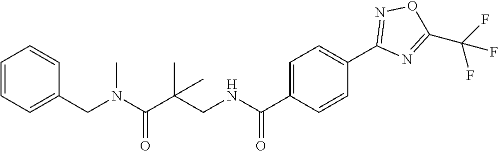 [SMILES: CN(Cc1ccccc1)C(=O)C(C)(C)CNC(=O)c1ccc(-c2noc(C(F)(F)F)n2)cc1] | 0.933 ± 0.26 | >15 |
| 6 | NT2-44 | 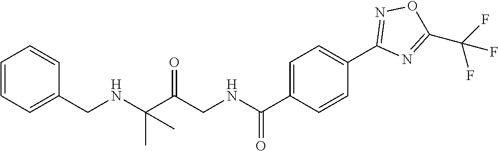 [SMILES: CC(C)(NCc1ccccc1)C(=O)CNC(=O)c1ccc(-c2noc(C(F)(F)F)n2)cc1] | 0.768 ± 0.19 | >15 |
| 7 | NT-174 | 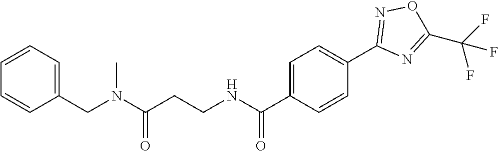 [SMILES: CN(Cc1ccccc1)C(=O)CCNC(=O)c1ccc(-c2noc(C(F)(F)F)n2)cc1] | 0.905 ± 0.22 | >15 |
| 8 | NT2-67 | 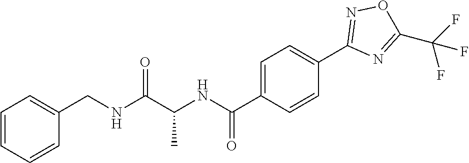 [SMILES: C[C@@H](NC(=O)c1ccc(-c2noc(C(F)(F)F)n2)cc1)C(=O)NCc1ccccc1] | 0.953 | >15 |
| 9 | NT-176 | 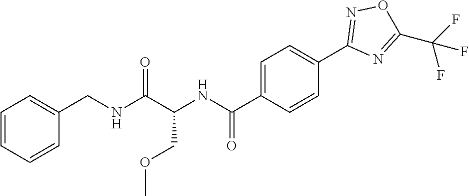 [SMILES: COC[C@@H](NC(=O)c1ccc(-c2noc(C(F)(F)F)n2)cc1)C(=O)NCc1ccccc1] | 0.537 ± 0.15 | >15 |
| 10 | NT-192 | 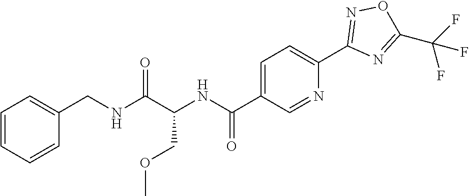 [SMILES: COC[C@@H](NC(=O)c1ccc(-c2noc(C(F)(F)F)n2)nc1)C(=O)NCc1ccccc1] | 1.710 ± 0.33 | >15 |
| 11 | NT2-156 | 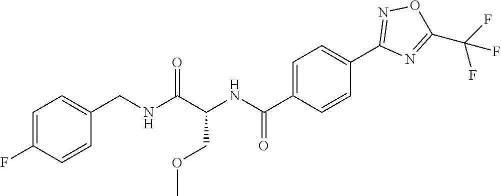 [SMILES: COC[C@@H](NC(=O)c1ccc(-c2noc(C(F)(F)F)n2)cc1)C(=O)NCc1ccc(F)cc1] | 0.233 ± 0.11 | >15 |
| 12 | NT2-191 | 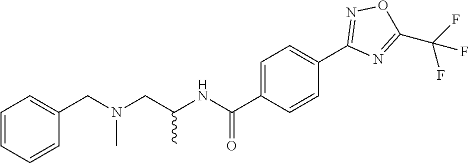 [SMILES: CC(CN(C)Cc1ccccc1)NC(=O)c1ccc(-c2noc(C(F)(F)F)n2)cc1] | 0.395 ± 0.14 | >50 |
| 13 | NT3-11D | 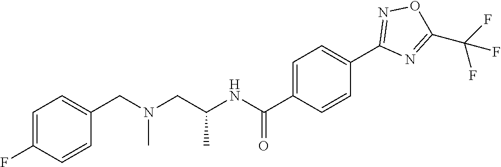 [SMILES: C[C@H](CN(C)Cc1ccc(F)cc1)NC(=O)c1ccc(-c2noc(C(F)(F)F)n2)cc1] | 0.059 ± 0.016 | >15 |
| 14 | NT3-89B | 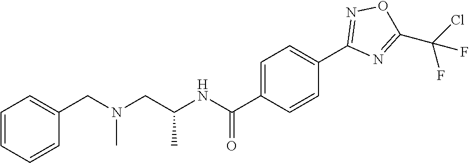 [SMILES: C[C@H](CN(C)Cc1ccccc1)NC(=O)c1ccc(-c2noc(C(F)(F)Cl)n2)cc1] | >2 | >2.0 |
| 15 | NT3-76 | 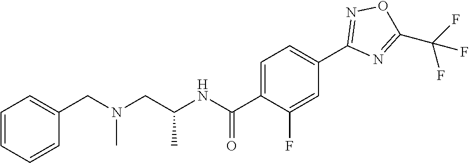 [SMILES: C[C@H](CN(C)Cc1ccccc1)NC(=O)c1ccc(-c2noc(C(F)(F)F)n2)cc1F] | 0.032 | >5.0 |
| 16 | NT3-131 | 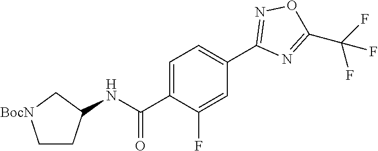 | ND | ND |
| 17 | NT3-131A | 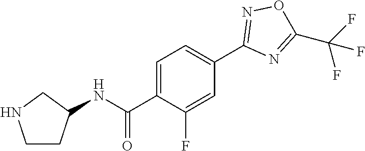 | ND | ND |
| 18 | NT3-75 | 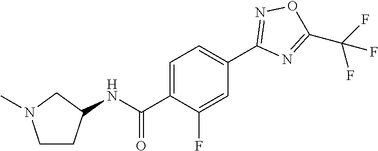 | 0.072 | >3.0 |
| 19 | NT3-83 | 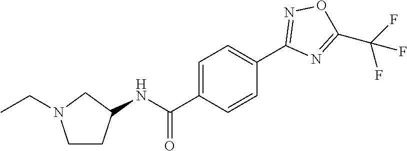 | 0.091 | >9.0 |
| 20 | NT3-81 | 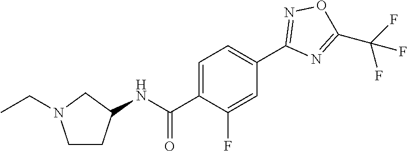 | 0.137 | >5.0 |
| 21 | NT3-123 | 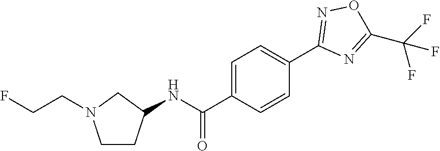 | >10 | >50 |
| 22 | NT3-92 | 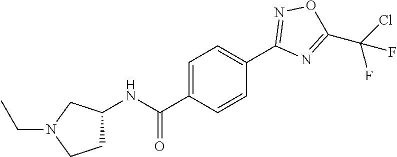 [SMILES: CCN1CC[C@@H](NC(=O)c2ccc(-c3noc(C(F)(F)Cl)n3)cc2)C1] | >20 | >50 |
| 23 | NT2-174 | 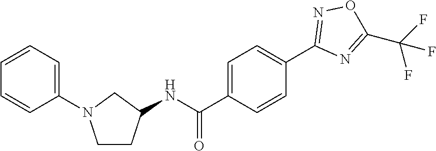 | >20 | >50 |
| 24 | NT2-160 | 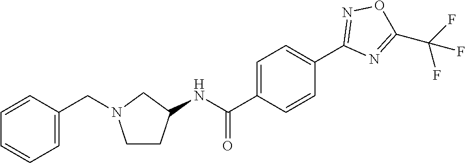 | 0.192 | >20 |
| 25 | NT3-40 | 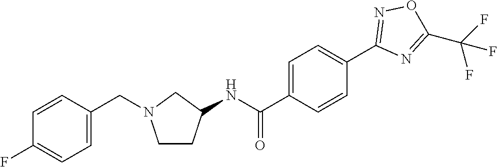 | 0.142 | >20 |
| 26 | NT3-132B | 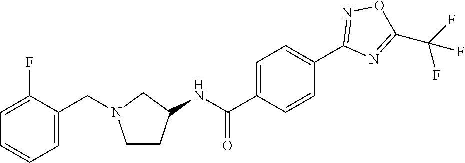 | 0.300 | >50 |
| 27 | NT3-132C | 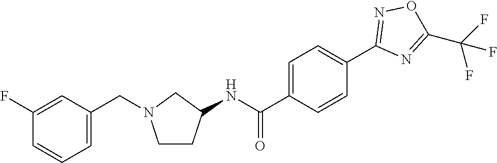 | ND | ND |
| 28 | NT3-77 | 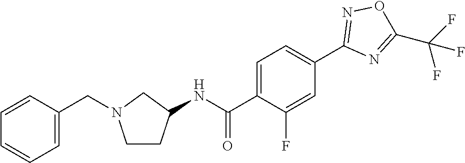 | 0.152 | >10 |
| 29 | NT3-77A | 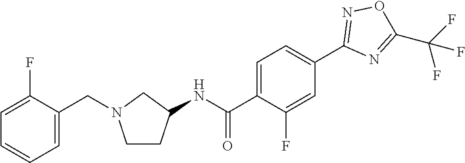 | 0.179 | >10 |
| 30 | NT3-77B | 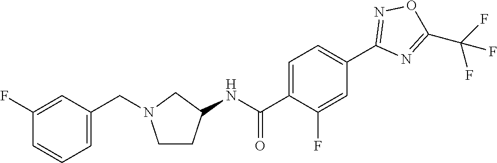 | 0.195 | >10 |
| 31 | NT3-77C | 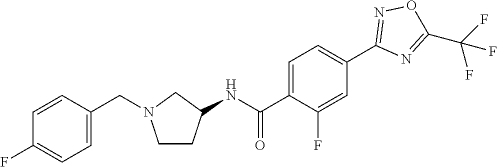 | 0.173 | >10 |
| 32 | NT3-39 | 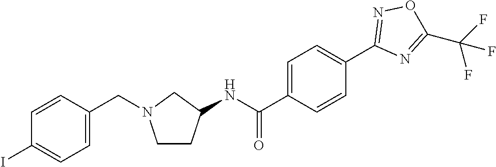 | 0.295 | >50 |
| 33 | NT3-127B | 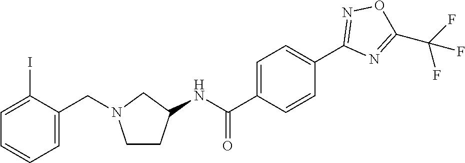 | 1.00 | >50 |
| 34 | NT3-106C | 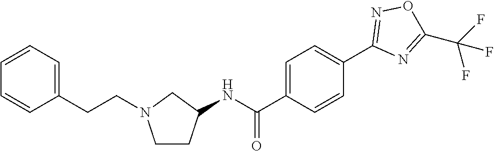 | >10 | >50 |
| 35 | NT3-102C | 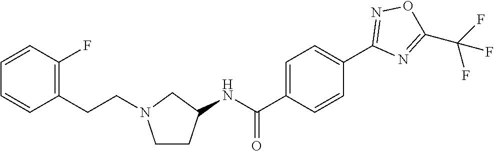 | >10 | >50 |
| 36 | NT3-52B | 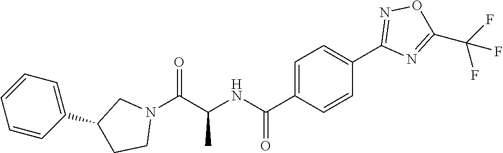 [SMILES: C[C@H](NC(=O)c1ccc(-c2noc(C(F)(F)F)n2)cc1)C(=O)N1CC[C@H](c2ccccc2)C1] | >1.00 | >50 |
| 37 | NT2-190 | 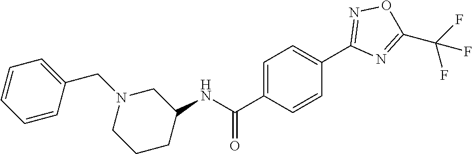 | >10 | >50 |
| 38 | NT3-16D | 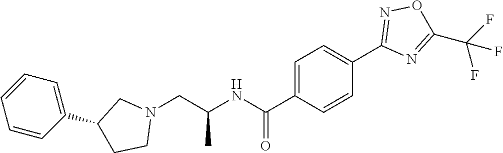 [SMILES: C[C@@H](CN1CC[C@H](c2ccccc2)C1)NC(=O)c1ccc(-c2noc(C(F)(F)F)n2)cc1] | 0.005 | >25 |

*ND: not determined

### Example 3. Radiofluorination of Bromodifluorooxadiazoles

### Radiochemistry

Recipe for the preparation of kryptofix/K₂CO₃ solution: K₂CO₃ (25 mg) and kryptofix (100 mg) were dissolved in acetonitrile (15.0 mL) and water (5.0 mL).

Recipe for the preparation of kryptofix/Cs₂CO₃ solution: Cs₂CO₃ (40 mg) and kryptofix (100 mg) were dissolved in acetonitrile (15.0 mL) and water (5.0 mL).

The solution of [¹⁸F] was purchased from NCM USA (Bronx, NY). The [¹⁸F] is trapped on a QMA cartridge and then eluted with 1.0-1.2 mL of a solution that contains kryptofix/K₂CO₃ or kryptofix/Cs₂CO₃ to a V-vial (Wheaton) with 92-96% recovery. The solvent was removed under a stream of Argon at 110° C. Water residue was removed aziotropically with the addition of acetonitrile (3×1.0 mL) and repeated drying under a stream of Argon at 110° C.

General example: A solution of the bromo-precursor (6-8 mg) in the appropriate solvent (i.e. DMSO) (0.4 mL) was added to the dried K[¹⁸F]/kryptofix or Cs[¹⁸F]/kryptofix and the mixture was heated at 150° C. for 20 minutes. The reaction mixture was cooled and passed through a silica gel cartridge (waters, 900 mg) and eluted with 30% methanol in dichloromethane (2.5 mL). After evaporating of the solvent under a stream of argon at 60-80° C., the residue was re-dissolved in the appropriate HPLC solvent and purified by semipreparative HPLC. The solvent was evaporated under reduced pressure. Each radioactive product was co-injected with an authentic non-radiolabeled compound into an analytical column to confirm its purity and identity. The radiochemical purity was >95% for all tracers.

### General Radiochemical Synthesis of TFMO Containing Molecules

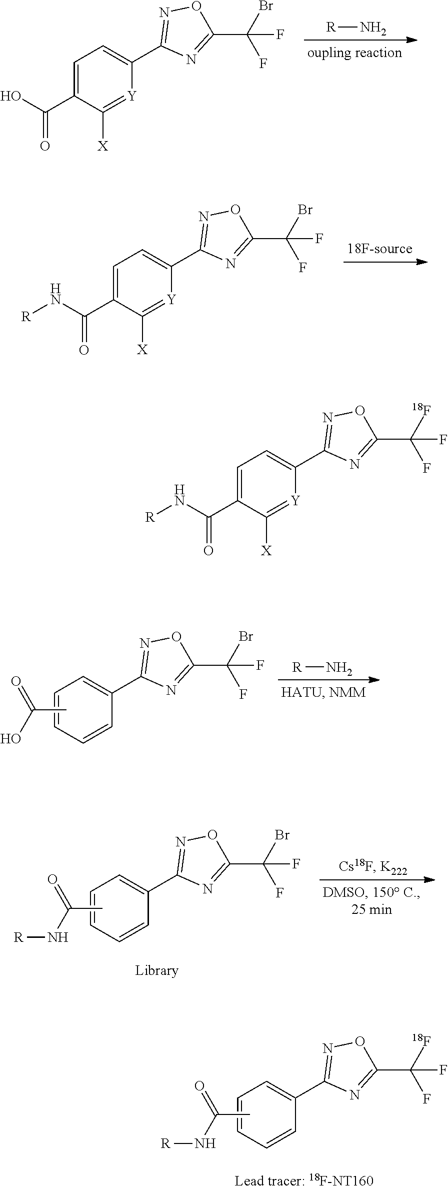

### Example of Radiochemical Synthesis of [¹⁸F] NT2-160

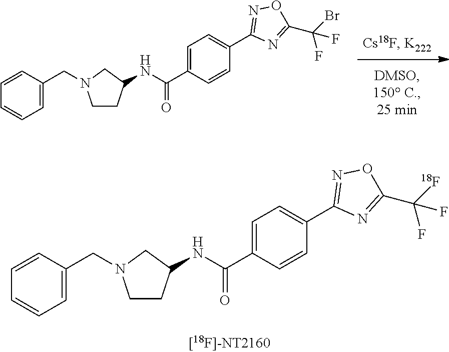

### Example of Radiochemical Synthesis of [¹⁸F] NT3-132B

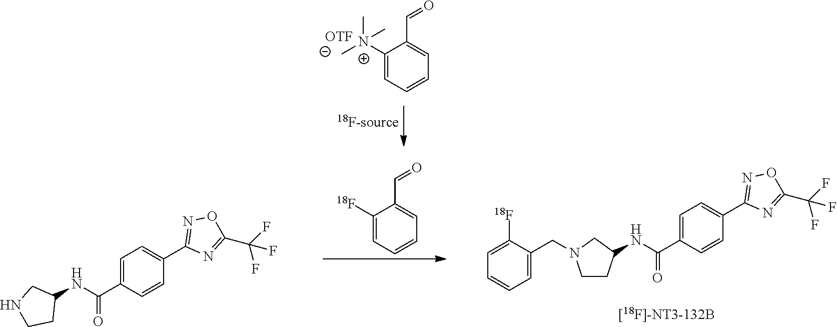

### General Radiochemical Synthesis of [¹¹C]-Containing Molecules

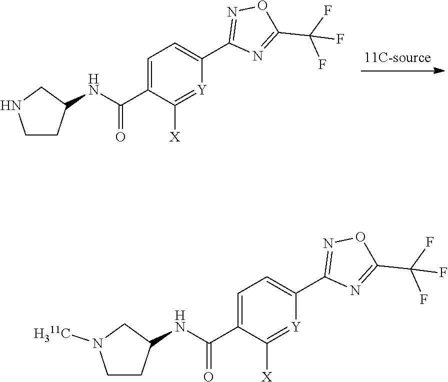

### Example of Radiochemical Synthesis of [¹¹C]-NT3-75

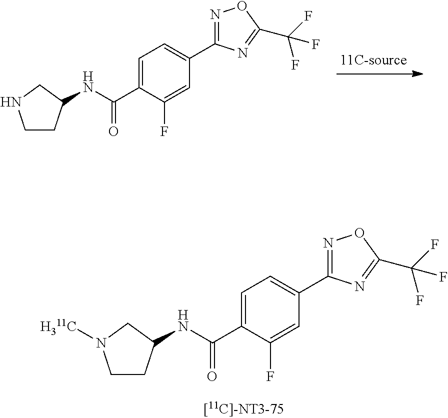

### Example of ¹⁸F-NT57

¹⁸F-NT57 was isolated with 75% acetonitrile/water solution in 17-19 minutes. The solvent was evaporated under reduced pressure and was re-dissolved in 20% ethanol/saline for animal injection. Each radioactive product was co-injected with an authentic non-radiolabeled compound into an analytical column to confirm its purity and identity. The radiochemical purity was >95% for all tracers.

Radiotracer formulation: The tracer was re-dissolved in 0-20% ethanol/saline for animal injection.

### Authentication of the Radioactive Tracers

The radioactive peak was detected with radioactivity detector co-injected with the relevant authentic cold compound which was detected with ultraviolet detector (254 nM) using analytical HPLC. The retention times for the radiotracers are:

¹⁸F-3 was eluted at 5.93 with 60% acetonitrile/water solution.

¹⁸F-4 was eluted at 6.0 with 60% acetonitrile/water solution.

¹⁸F-19 was eluted at 6.8 with 70% acetonitrile/water solution.

¹⁸F-20i was eluted at 6.2 with 70% acetonitrile/water solution.

¹⁸F-1 was eluted at 1.8 with 50% acetonitrile/water solution.

¹⁸F-2 was eluted at 1.8 with 50% acetonitrile/water solution.

### Specific Activity

The specific activity was determined from the area under the curve of the tracer attributed ultraviolet peak in the HPLC chromatogram against a calibration curve pre-prepared with the unlabeled reference standard. Specific activity of tracers ranged from 0.8 to 1.2 Ci/μmol. It is important to note, F-18 was purchased from a commercial source with significant decay prior to the start of radiochemical experiment. significantly higher specific activities for tracers were expected once cyclotron becomes operational. Furthermore, automated synthesis expected to further improve the radiochemical yield and specific activity due to more efficient synthesis and shorter overall production time. Moreover, starting with higher amount of radioactivity may also, further improve the yield and specific activities.

### Partition Coefficient (Log P)

Log P for ¹⁸F-NT57 was determined using the method similar to previous work (Turkman N, S. A., Paolillo V, et al. 2012). The log P was determined by partitioning the tracer between octanol and phosphate buffer at pH 7.4 and measuring the concentration of the tracer in each layer. The radioactivity of each layer is counted using gamma-counter. The partition coefficient (P) is calculated as [radioactivity (cpm/ml) in 1-octanol)]/[(radioactivity (cpm/ml) in phosphate buffer pH 7.4].

Precursors prepared by the current invention are shown in Table 2 below.

TABLE 2
List of precursors used for radiolabeling.

| | |
|---|---|
| NT-6 | 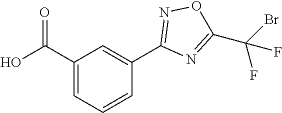 [SMILES: O=C(O)c1cccc(-c2noc(C(F)(F)Br)n2)c1] |
| NT-191 | 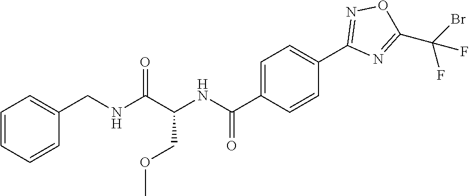 [SMILES: COC[C@@H](NC(=O)c1ccc(-c2noc(C(F)(F)Br)n2)cc1)C(=O)NCc1ccccc1] |
| NT-189 | 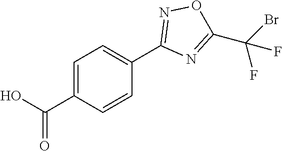 [SMILES: O=C(O)c1ccc(-c2noc(C(F)(F)Br)n2)cc1] |
| NT-192 | 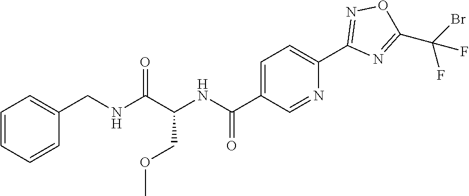 [SMILES: COC[C@@H](NC(=O)c1ccc(-c2noc(C(F)(F)Br)n2)nc1)C(=O)NCc1ccccc1] |
| NT-131 | 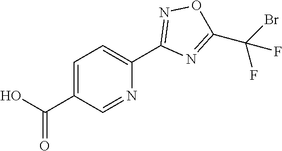 [SMILES: O=C(O)c1ccc(-c2noc(C(F)(F)Br)n2)nc1] |
| NT2- 66 | 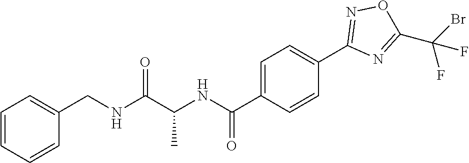 [SMILES: C[C@@H](NC(=O)c1ccc(-c2noc(C(F)(F)Br)n2)cc1)C(=O)NCc1ccccc1] |
| NT- 9A | 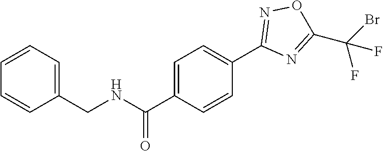 [SMILES: O=C(NCc1ccccc1)c1ccc(-c2noc(C(F)(F)Br)n2)cc1] |
| NT2- 129 | 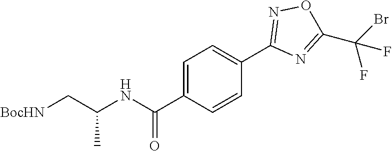 [SMILES: C[C@H](CNC(=O)OC(C)(C)C)NC(=O)c1ccc(-c2noc(C(F)(F)Br)n2)cc1] |
| NT- 9B | 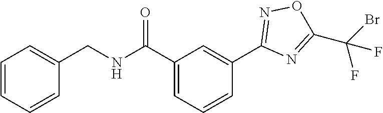 [SMILES: O=C(NCc1ccccc1)c1cccc(-c2noc(C(F)(F)Br)n2)c1] |
| NT2- 148 | 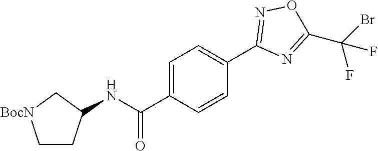 |
| NT- 73 | 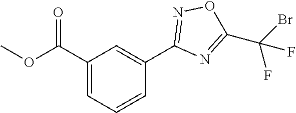 [SMILES: COC(=O)c1cccc(-c2noc(C(F)(F)Br)n2)c1] |
| NT2- 153 | 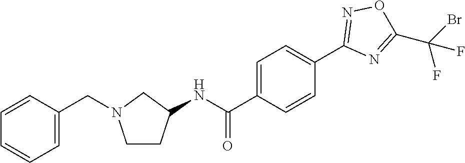 |
| NT- 75 | 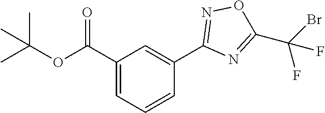 [SMILES: CC(C)(C)OC(=O)c1cccc(-c2noc(C(F)(F)Br)n2)c1] |
| NT2- 174Br | 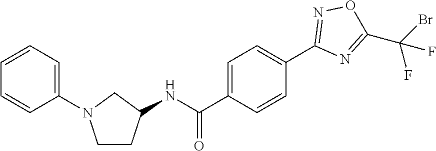 |
| NT3- 65B | 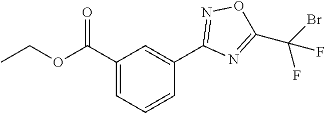 [SMILES: CCOC(=O)c1cccc(-c2noc(C(F)(F)Br)n2)c1] |
| NT2- 189 | 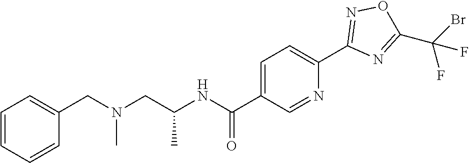 [SMILES: C[C@H](CN(C)Cc1ccccc1)NC(=O)c1ccc(-c2noc(C(F)(F)Br)n2)nc1] |
| NT2- 193B | 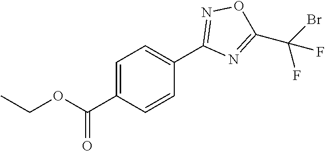 [SMILES: CCOC(=O)c1ccc(-c2noc(C(F)(F)Br)n2)cc1] |
| NT2- 191Br | 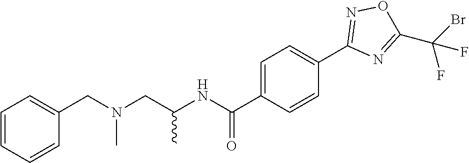 [SMILES: CC(CN(C)Cc1ccccc1)NC(=O)c1ccc(-c2noc(C(F)(F)Br)n2)cc1] |
| NT2- 197 | 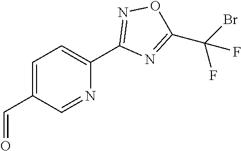 [SMILES: O=Cc1ccc(-c2noc(C(F)(F)Br)n2)nc1] |
| NT3- 16E | 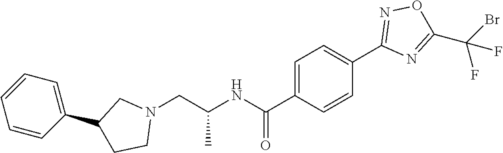 |
| NT3- 59B | 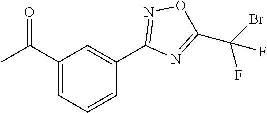 [SMILES: CC(=O)c1cccc(-c2noc(C(F)(F)Br)n2)c1] |
| NT3- 52C | 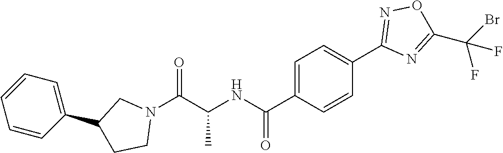 |
| NT3- 73 | 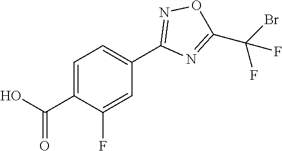 [SMILES: O=C(O)c1ccc(-c2noc(C(F)(F)Br)n2)cc1F] |
| NT3- 40Br | 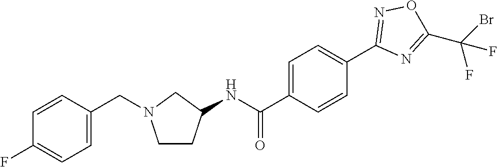 |
| NT- 57 | 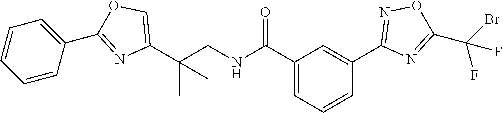 [SMILES: CC(C)(CNC(=O)c1cccc(-c2noc(C(F)(F)Br)n2)c1)c1coc(-c2ccccc2)n1] |
| NT3- 90 | 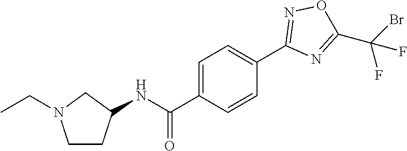 |
| NT- 160Br | 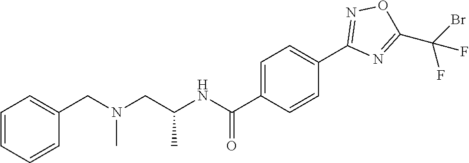 [SMILES: C[C@H](CN(C)Cc1ccccc1)NC(=O)c1ccc(-c2noc(C(F)(F)Br)n2)cc1] |
| NT3- 81 | 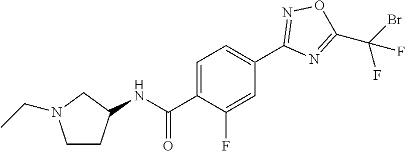 |
| NT3- 131A | 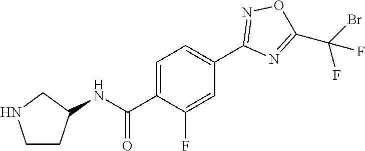 |
| NT3- 77 | 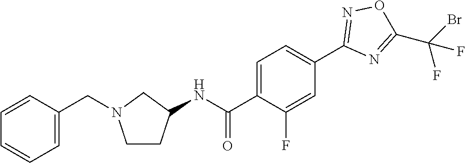 |
| NT3- 75Br | 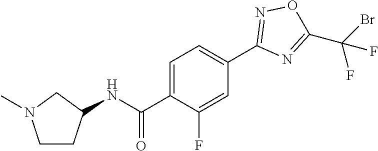 |

The radioactive tracers used for PET imaging are showing in the tale below.

TABLE 3
List of radioactive tracers.

| | Structure | | Structure |
|---|---|---|---|
| ID | (Radioactive) | ID | (Radioactive) |
| [¹⁸F] NT-6 | 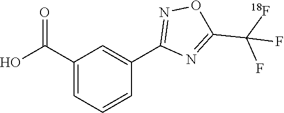 [SMILES: O=C(O)c1cccc(-c2noc(C(F)(F)[18F])n2)c1] | [¹⁸F] NT-176 | 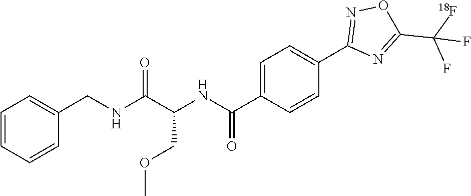 [SMILES: COC[C@@H](NC(=O)c1ccc(-c2noc(C(F)(F)[18F])n2)cc1)C(=O)NCc1ccccc1] |
| [¹⁸F] NT-189 | 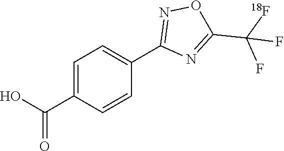 [SMILES: O=C(O)c1ccc(-c2noc(C(F)(F)[18F])n2)cc1] | [¹⁸F] NT-192 | 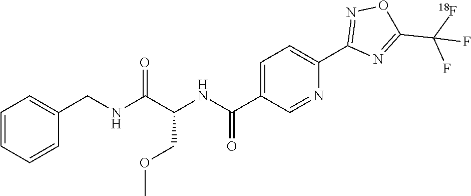 [SMILES: COC[C@@H](NC(=O)c1ccc(-c2noc(C(F)(F)[18F])n2)nc1)C(=O)NCc1ccccc1] |
| [¹⁸F] NT-9A | 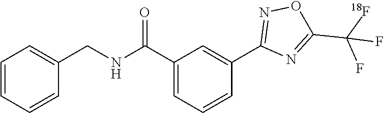 [SMILES: O=C(NCc1ccccc1)c1cccc(-c2noc(C(F)(F)[18F])n2)c1] | [¹⁸F] NT2-67 | 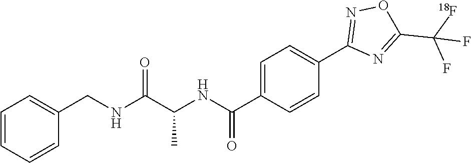 [SMILES: C[C@@H](NC(=O)c1ccc(-c2noc(C(F)(F)[18F])n2)cc1)C(=O)NCc1ccccc1] |
| [¹⁸F] NT-9B | 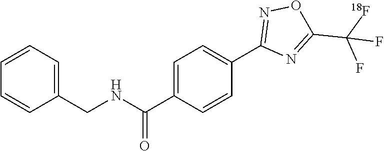 [SMILES: O=C(NCc1ccccc1)c1ccc(-c2noc(C(F)(F)[18F])n2)cc1] | [¹⁸F] NT2-162 | 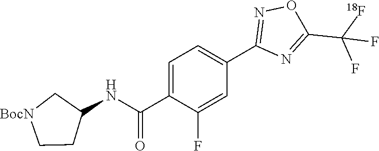 |
| [¹⁸F] NT-193B | 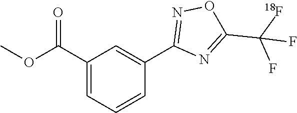 [SMILES: COC(=O)c1cccc(-c2noc(C(F)(F)[18F])n2)c1] | [¹⁸F] NT2- 162A | 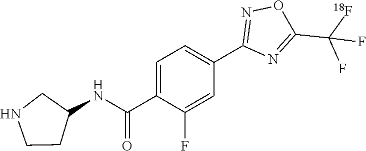 |
| [¹⁸F] NT3-65A | 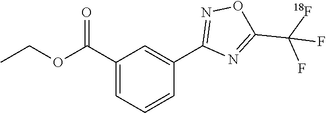 [SMILES: CCOC(=O)c1cccc(-c2noc(C(F)(F)[18F])n2)c1] | [¹⁸F] NT2-160 | 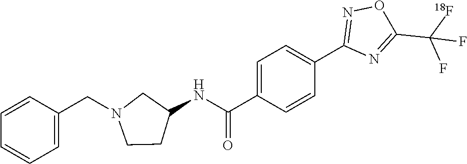 |
| [¹⁸F] NT-195A | 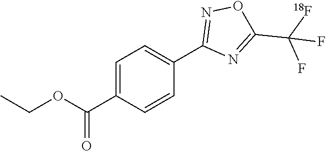 [SMILES: CCOC(=O)c1ccc(-c2noc(C(F)(F)[18F])n2)cc1] | [¹⁸F] NT3-16D | 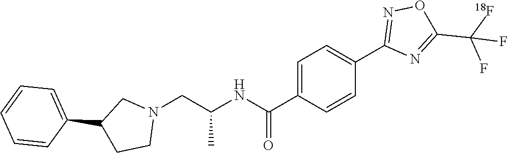 |
| [¹⁸F] NT-57 | 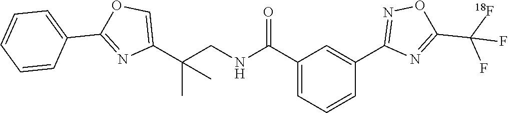 [SMILES: CC(C)(CNC(=O)c1cccc(-c2noc(C(F)(F)[18F])n2)c1)c1coc(-c2ccccc2)n1] | [¹⁸F] NT3-83 | 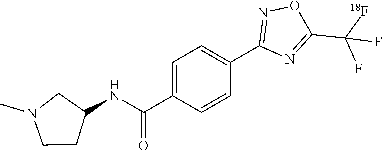 |
| [¹⁸F] NT-160 | 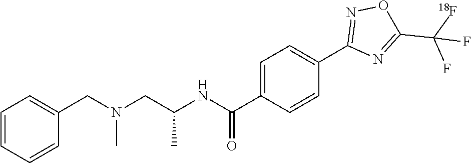 [SMILES: C[C@H](CN(C)Cc1ccccc1)NC(=O)c1ccc(-c2noc(C(F)(F)[18F])n2)cc1] | [¹⁸F] NT3-81 | 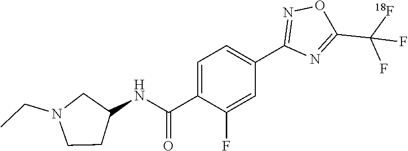 |
| [¹⁸F] NT3-77 | 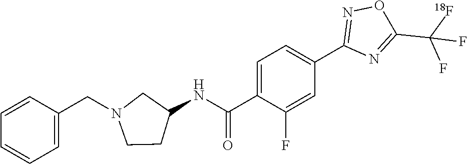 | [¹⁸F] NT3-75 | 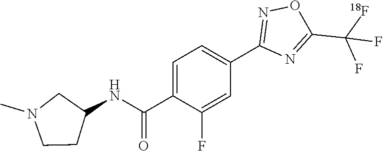 |
| [¹⁸F] NT3-77A | 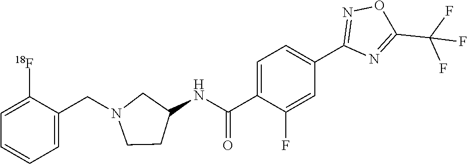 | [¹¹C] NT3-83 | 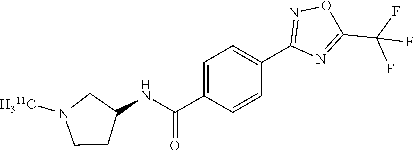 |
| [¹⁸F] NT3-77B | 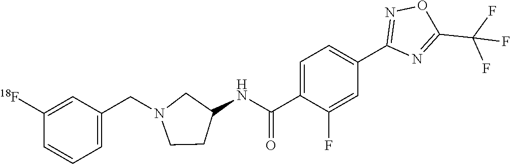 | [¹¹C] NT3-131 | 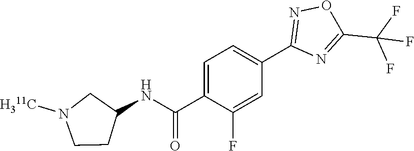 |
| [¹⁸F] NT3-77A | 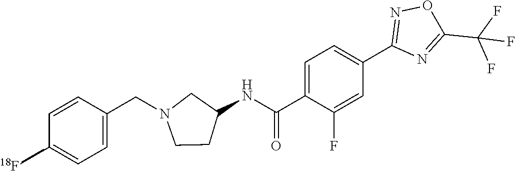 | [¹⁸F] NT3-132b | 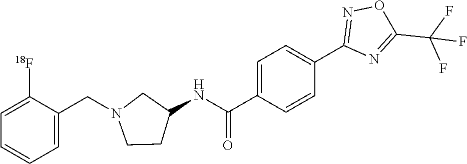 |
| [¹⁸F] NT3-40 | 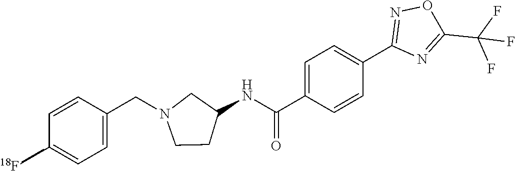 | [¹⁸F] NT3- 132C | 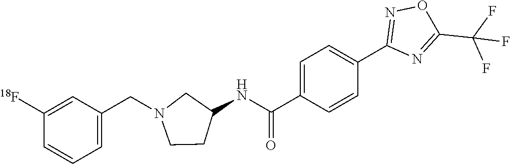 |

### Example 4. PET Imaging Using Radio Labeled Homologs

In Vivo microPET (uPET) Imaging of Healthy Rats

Sprague Dawley rats (200-250 g, N=3,) were anesthetized with 3% isoflurane in oxygen and maintained at 2% isoflurane in oxygen throughout the imaging studies. Anesthetized rat was placed in the uPET (Siemens, Knoxville, TN) with the skull positioned in the center of the field of view. The tracer (500-700 μCi/animal) was administered intravenously into each Rat (n=3 per group) in a total volume 0.5-1.0 ml solution. Dynamic PET images were obtained over 60 minutes. Images were reconstructed with attenuation correction using an ordered subset expectation maximization (OSEM2D) algorithm with 16 subsets and 4 iterations.

### PET Image Data Analysis

Dynamic PET images were analyzed with Inveon Research Workplace 4.0 (Siemens, Knoxville, TN) and AMIDE software using manual segmentation of regions of interest (ROI), were drawn over ROI on the decay-corrected coronal images. The radioactivity accumulation (tracer uptake) within the ROI were obtained as percentage of the injected dose (ID) per gram of tissue (% ID/g). The time-activity curve (TAC) was generated from the region of interest (ROI) by plotting the radioactivity/cc vs time.

Further studies are warranted to determine the extent of the metabolic profile of ¹⁸F-NT57 in the blood and brain tissues. It is important to note that other PET tracer candidates identified from extensive structure activity relationship studies are undergoing preclinical evaluation in the laboratory to optimize the in vivo profile (i.e. increasing BBB permeability and tracer uptake in the brain). The new data will be published in due course.

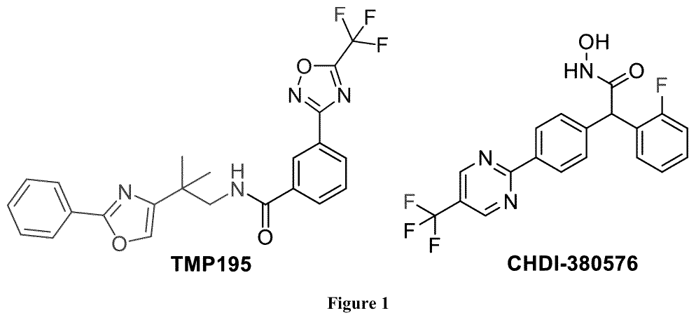

The TFMO bearing molecules are an attractive target for PET tracer development and SAR studies because they contain the three distinctive HDAC pharmacophore motifs (color coded, FIG. 1): cap moiety (green) that interacts with the surface of the HDAC enzyme; linker moiety (black), that occupies a hydrophobic channel and the TFMO moiety (blue) that interacts with the zinc ion at the bottom of the class-lla HDAC catalytic pocket (AI-Sanea, M. M., et al., 2020; Chen, Y., et al., 2019; Raudszus, R., et al., 2019). Given the specific interest in targeting class-lla HDACs for PET tracer development, the TFMO-bearing molecules met the criteria for PET probe development such as high specificity to class-lla HDACs, and accessibility for late stage single-step incorporation of the F-18. Due to the short half-life of the F-18 labeled PET radiotracers, it is highly desired to design a late stage labeling site that allows for radiolabeling molecules in a short time that produce sufficient radiochemical yield with high radiochemical purity and suitable specific activity.

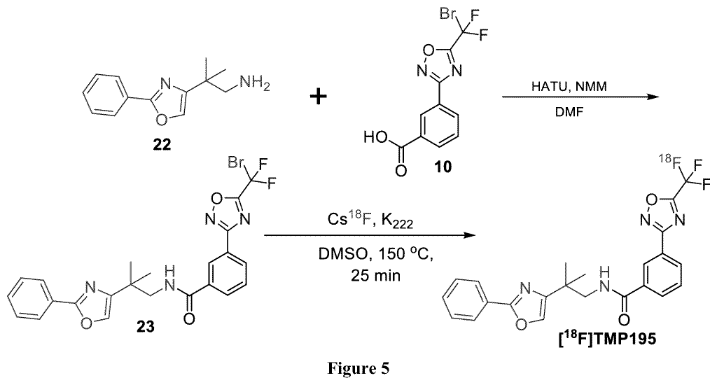

A radiochemical route (Scheme 1) was designed to radiolabel the trifluoromethyl-oxadiazole (TFMO) moiety with F-18 and it was utilized to radiolabel TMP195 with F-18 as shown in Scheme 3 (FIG. 5). The strategy was to incorporate the F-18 in the exact location as it appears on the TFMO moiety of class-lla HDAC inhibitors, thus enabling the radiosynthesis of ¹⁸F-NT57 as an identical radioactive homolog of TMP195 maintaining its exact affinity and specificity to class-lla HDACs. This approach also enabled a highly desirable one-step radiolabeling reaction which can facilitate automated routine production of tracers in hot cells used in clinical setups. This is also by design a universal labeling site that allows for radiolabeling many molecules without changing the radiolabeling method. Moreover, the universal labeling site allows for extensive in vivo SAR studies to delineate and optimize the in vivo characteristics of the lead PET tracer (i.e. metabolic and pharmacokinetic profile) without changing the radiolabeling method or radiolabeling site (Van de Bittner, G. C., E. L. Ricq, and J. M. Hooker, 2014).

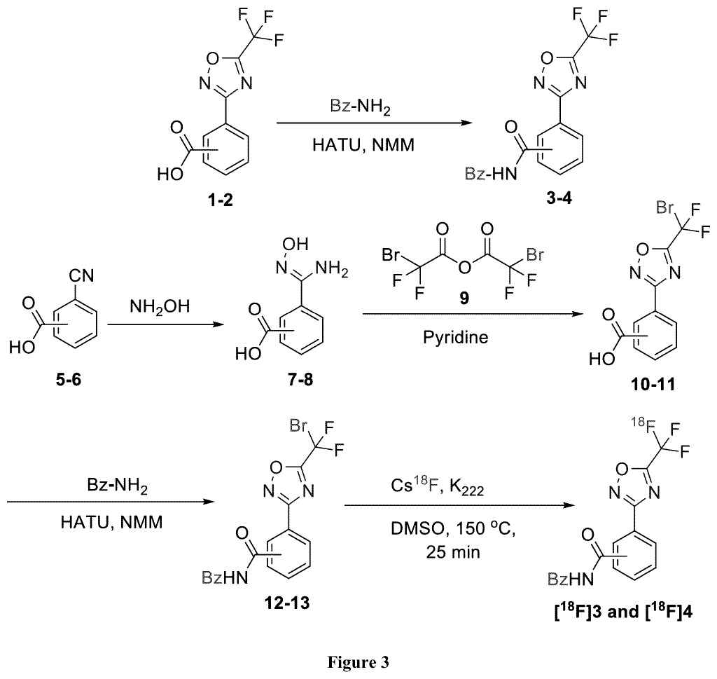

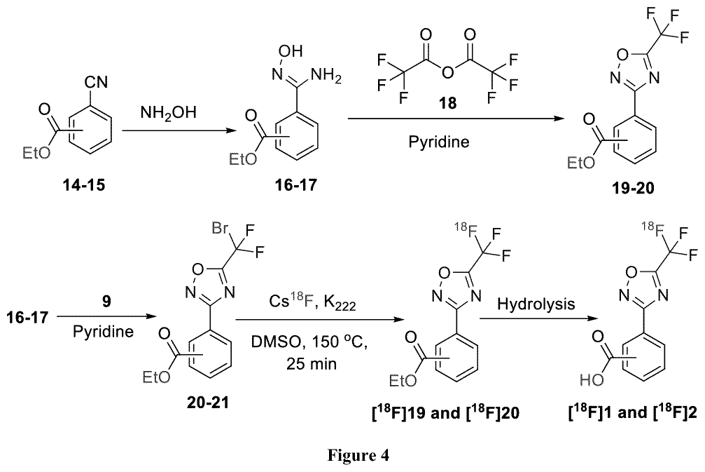

The chemical synthesis and radiolabeling reactions of the TFMO containing molecules are shown in Schemes 1 and 2 (FIGS. 3 and 4). TFMO containing amides were the starting material to mimic the class-lla HDAC inhibitors (Scheme 1). Simple amides were selected for simplicity and ease of synthesis. The chemical syntheses of the TFMO containing amides started by coupling the commercially available 3- or 4-(5-(trifluoromethyl)-1,2,4-oxadiazol-3-yl) benzoic acid (1-2) with benzylamine in the presence of equivalent amounts of 1-[Bis(dimethylamino)methylene]-1H-1,2,3-triazolo[4,5-b]pyridinium 3-oxid hexafluorophosphate (HATU) and excess of 4-methylmorpholine (NMM) to afford the reference standard TFMO amides: N-benzoyl-3 or 4-(5-(trifluoromethyl)-1,2,4-oxadiazol-3-yl)benzamide 3 (meta-isomer) or 4 (para-isomer) respectively. The preparation of the essential precursors (12-13) for radiochemistry started by heating 3- or 4-cyanobenzoate (5 or 6) under reflux with hydroxylamine to afford (Z)-3- or 4-(N′-hydroxycarbamimidoyl)benzoate (7 or 8) in quantitative yields and was then treated with bromodifluoroacetic anhydride (9) in pyridine to afford the 3- or 4-(5-(bromodifluoromethyl)-1,2,4-oxadiazol-3-yl) benzoate (10 or 11) in 70-80% yield. 10 or 11 were coupled with the benzyl amine similar to 1-2 to afford the bromo-precursors: N-benzoyl-3 or 4-(5-(bromodifluoromethyl)-1,2,4-oxadiazol-3-yl) benzamide (12 or 13) in 60-70% yield which sets the stage for subsequent radiolabeling reactions.

The radiochemistry was performed initially by treating the bromodifluoromethyl-precursors 12 or 13 with potassium ¹⁸F-fluoride (K¹⁸F) to produce the ¹⁸F-TFMO containing tracers: ¹⁸F-3 or ¹⁸F-4 with very high radiochemical purity (>98%), albeit the radiochemical yield was very low (<0.5%). Remarkably, using cesium ¹⁸F-fluoride (Cs¹⁸F) led to a significantly improved radiochemical yield of 2-5% (decay corrected).

To extend the utility of the new radiochemical design, TFMO containing esters with F-18 was radiolabeled using the same strategy. Also, the radioactive acids ¹⁸F-1 and ¹⁸F-2 were generated which are expected to be generated in vivo from cleavage of the amide bond of the class-lla HDAC inhibitors (i.e. ¹⁸F-NT57) by the metabolizing enzymes. As such, the acid tracers could be useful to determine the metabolic profile of the new tracers in vivo.

The chemical synthesis was performed similar to Scheme 1 above. The commercially available ethyl 3- or 4-cyanobenzoate 14 or 15 was heated under reflux with hydroxylamine to afford (Z)-ethyl 3 or 4-(N′ hydroxycarbamimidoyl) benzoate (16-17) in quantitative yield and was then treated with trifluoroacetic anhydride (18) to afford the ethyl 3 or 4-(5-(trifluoromethyl)-1,2,4-oxadiazol-3-yl) benzoate (19-20) in 70-80% yield. Similarly, the bromo-precursor analogs ethyl 3- or 4-(5-(bromodifluoromethyl)-1,2,4-oxadiazol-3-yl) benzoate (20-21) was synthesized by reacting the oximes 16-17 with the bromodifluoroacetic anhydride (9) as shown in Scheme 2 (FIG. 4). Then 20 or 21 was reacted with Cs¹⁸F at 150° C. in DMSO to successfully generate the desired PET tracer ¹⁸F-19 or ¹⁸F-20 in 2-5% radiochemical yield (decay corrected). Hydrolysis of ¹⁸F-19 or ¹⁸F-20 using 1.0 N NaOH afforded the acids ¹⁸F-1 or ¹⁸F-2 in quantitative yield.

Despite the attempts to improve the radiochemical yield by varying the solvents and temperature as summarized in Table 1, dimethyl sulfoxide (DMSO) at 150° C. gave the best radiochemical yield. N, N-dimethylacetamide (OMA) gave slightly lower radiochemical yield compared to DMSO, however, the reaction seems to be cleaner (less radioactive and non-radioactive impurities). The use of acetonitrile failed to yield any product. As a result, DMSO was selected as the solvent of choice for subsequent radiolabeling reactions of class-lla HDAC inhibitors. Notably, no reaction occurred below 145° C. and temperatures higher than 160° C. produced lower radiochemical yields. The use of microwave technology is explored to further improve the radiochemical yield.

TABLE 4
Optimization of the conditions for the
¹⁸F-incorporatation into the TFMO moiety.

| Entry | Base | Solvent | Temp. (° C.) | Time | % RCY |
|---|---|---|---|---|---|
| 1 | K₂CO₃ | DMSO | 150 | 25 | <0.5 |
| 2 | Cs₂CO₃ | DMSO | 150 | 25 | 3-5 |
| 3 | Cs₂CO₃ | DMF | 150 | 25 | 0 |
| 4 | Cs₂CO₃ | DMA | 150 | 25 | 3-5 |
| 5 | Cs₂CO₃ | Acetonitrile | 150 | 25 | 0 |

Next, the utility of novel radiochemical design to produce the ¹⁸F-TFMO containing class-lla HDAC inhibitors were examined. The radiosynthesis of ¹⁸F-NT57, a radioactive homolog of TMP195 is outlined in Scheme 3 (FIG. 5). 3-(5-(bromodifluoromethyl)-1,2,4-oxadiazol-3-yl)-N-(2-methyl-2-(2-phenyloxazol-4-yl)propyl) benzamide (23) was synthesized similar to the prior synthesis of 12-13. Compound 10 was coupled with the 2-methyl-2-(2-phenyloxazol-4-yl) propan-1-amine (22) using HATU and NMM to generate the bromo-precursor (23) in 50-60% yield. Then compound 23 was reacted with Cs¹⁸F at 150° C. in OMA or DMSO to successfully generate the desired class-lla HDAC targeting PET tracer ¹⁸F-NT57 in 2-5% radiochemical yield (decay corrected). It is critical to note that despite the relatively low radiochemical yield, sufficient radioactivity was obtained to readily and routinely perform preclinical imaging studies even when starting with as low as 150 mCi.

Owing to the single step radiosynthesis, a radioactive dose sufficient for multiple human studies (typically ˜5.0-10.0 mCi/subject) can be readily produced starting with ˜1.0 Ci of F-18 which can be easily generated in most cyclotron facilities. In fact, due to the shorter time of the radiosynthesis and dose formulation, it is likely that performing the radiosynthesis using a fully automated module will significantly improve the amount of the final dose. Notably, the yield was sufficient for performing preclinical studies and will also be sufficient to produce a clinical dose for human studies, if a TFMO based tracer from the studies becomes available for clinical translation. Also, other sites for radiolabeling are currently being pursued to improve the radiochemical yield while preserving the high affinity to class-lla HDACs. PET imaging of class-lla HDACs, an important target for cancer and brain imaging obtained with the ¹⁸F-TFMO-containing molecules underscores the significance of the radiochemical labeling method reported in this invention. In addition to the radiolabeled class-lla HDAC inhibitors, it is likely that the radiochemical methods reported herein can be extended to other classes of trifluoromethyl containing heterocyclic small molecules that appear in inhibitors of highly pursued imaging targets such as cyclooxygenases (Cox-1 and Cox-2) and estrogen receptors (Kumar, J. S. D., et al., 2018; Long, S., et al. 2009; Zhang, S., et al., 2017).

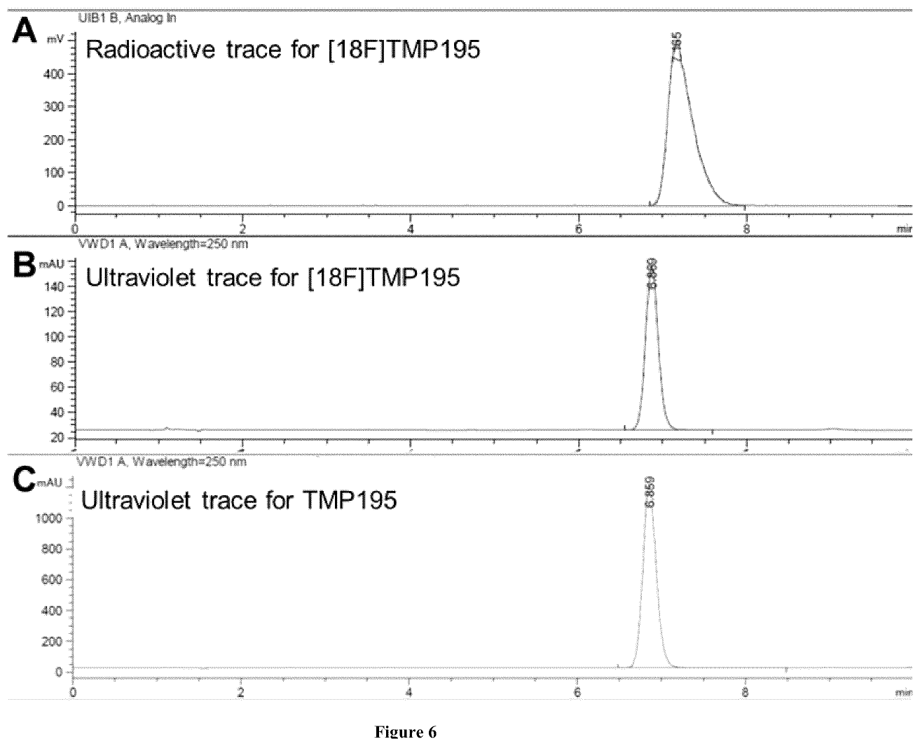

The new tracer was purified using a semi-preparative HPLC system (C18 column with 4.0 ml/min flow rate using 75% acetonitrile/water). The overall radiochemical yield of ¹⁸F-NT57 was within 2-5% (end of radiosynthesis, decay-corrected, n=10) and the radiochemical purity was >98%. The identity of ¹⁸F-NT57 was confirmed by co-injection together with the corresponding cold (unlabeled) molecule using analytical HPLC as shown in FIG. 6 (chromatograms a and c). The specific activity of ¹⁸F-NT57 was determined from the area under the curve of the tracer attributed to ultraviolet peak in the HPLC chromatogram (FIG. 6, chromatograms 8) against a calibration curve pre-prepared with the unlabeled reference standard. The specific activity ranged from 1 to 1.2 mCi/nmol. The specific activity is expected to improve significantly once onsite cyclotron becomes operational.

Currently, the F-18 is purchased from an outside source, as a result it undergoes significant decay (several cycles) before reaching the laboratory which reduces the overall specific activity. The radiosynthesis, HPLC purification and formulation of the final radioactive dose was accomplished manually in less than 100 min. The partition coefficients (Log D) using the octanol/PBS shake flask method was determined (Turkman, N., et al., 2012). The observed Log D value of ¹⁸F-NT57 was >6.0 which is expected for such a highly lipophilic compound.

To further validate the utility of the new radiochemical approach in vivo, PET imaging with ¹⁸F-NT57 in healthy Sprague Dawley rats was performed.

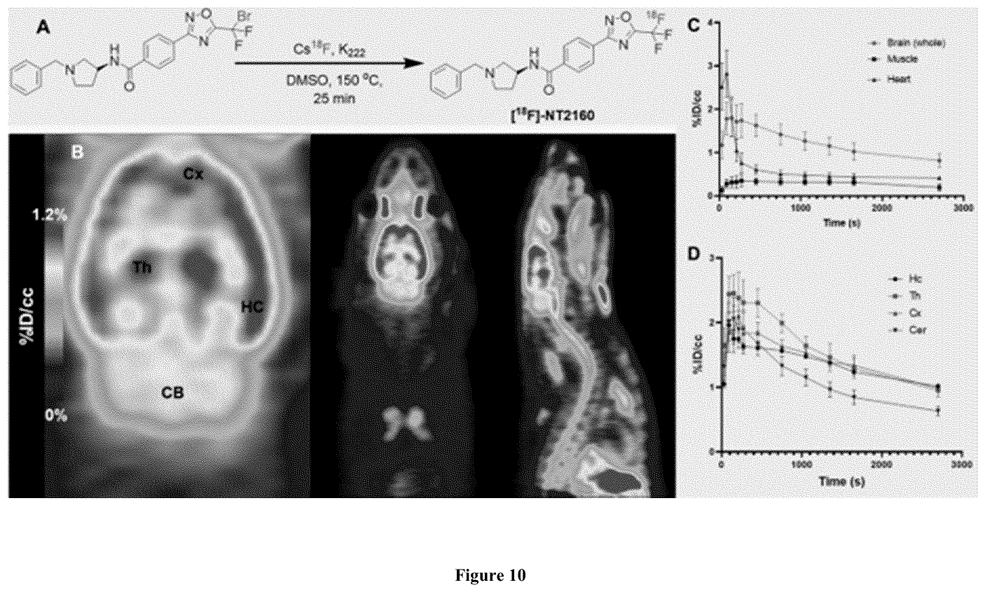

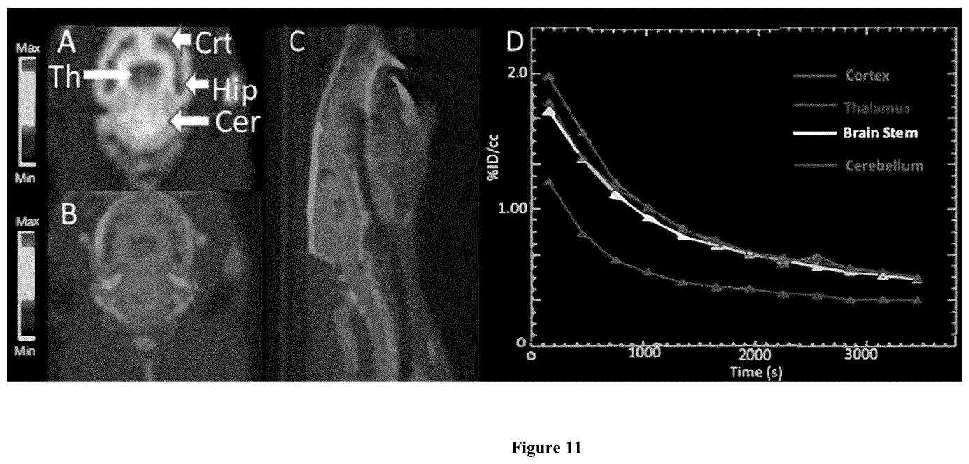

Anesthetized Sprague Dawley rats (200-250 g, N=6) were placed in the Inveon uPET (Siemens, Knoxville, TN) in the supine position with the skull positioned in the center of the field of view. [¹⁸F]-NT160 was formulated in ethanol:polysorbate 80:saline (10:10:80%) and it was stable in this formulation at 37° C. at least for 4 hours. [¹⁸F]-NT160 (18.5-25.9 MBq/animal) was administered via the tail-vein injection in a total volume 0.5-1.0 ml. Dynamic PET images were obtained over 60 minutes followed by CT imaging (15 min scan). Images were reconstructed with attenuation correction using an ordered subset expectation maximization (OSEM2D) algorithm with 16 subsets and 4 iterations. PET image analysis was performed using the AMIDE software by using manual segmentation of regions of interest (ROI), including: whole brain, cortex, hippocampus, thalamus, cerebellum, and brain stem. Rat Brain Atlas was used for alignment and identification of specific anatomical markers of the brain. The time-activity curve (TAC) was generated from the region of interest (ROI) by plotting the radioactivity/cc vs time. Levels of accumulation of the radiotracer in tissues were expressed as standard uptake values (SUV) that were calculated for the regions of interest (ROI) using the AMIDE software. The SUV is defined as the ratio of the tissue radioactivity concentration C (e.g. expressed as Bq/g tissue) at given time point post injection T, and the injected dose (e.g. in Bq, decay-corrected to the same time T), and normalized by the body weight in grams. GraphPad Prism (Graph Pad Software La Jolla, CA) was used for image data analysis. [¹⁸F]-NT2-160 was also prepared by the same or similar procedure. Their PET imaging results were shown in FIGS. 10 and 11.

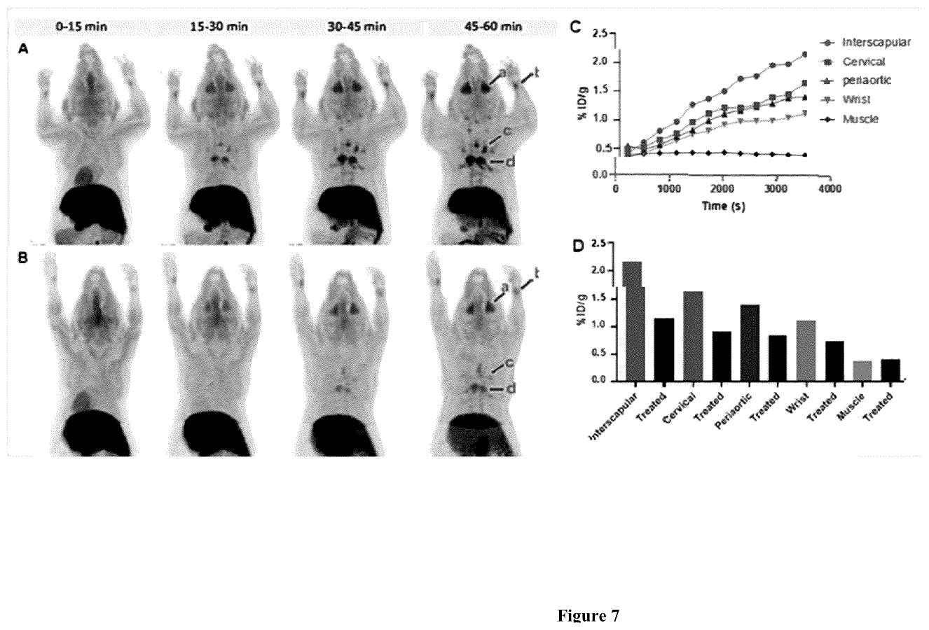

Dynamic PET imaging studies also provided real time information on the biodistribution of ¹⁸F-NT57 as a surrogate to TMP195 in rat tissues over 60 min as summarized in FIG. 7 (A). ¹⁸F-NT57 displayed rapid clearance from most normal tissues (i.e. heart, brain, muscle) but accumulated heavily in the liver as expected for such a highly lipophilic compound (Log D>6.0) and further excreted by hepatobiliary elimination. ¹⁸F-NT57 displayed intense and increased uptake in the brown fat adipose tissues (BATs). The specific uptake in BATs was assigned according to the report by Mukherjee J. et. al. (Schade, K. N., et al., 2015). It was confirmed that the specificity of the tracer uptake in these tissues by co-administering ¹⁸F-NT57 with a cold dose of TMP195 (2 mg/kg). The reduced tracer uptake in BATs is clearly visible as demonstrated by the PET images (FIG. 7, B). Quantitative data obtained from the time activity curve (TAC) over 60 min demonstrated an increase in tracer uptake in both the cervical and interscapular BAT tissues (FIG. 7, C). Interestingly, PET data obtained with this tracer is similar to the PET imaging data obtained with ¹⁸F-FDG in BATs activated with CL316,243, a beta3-adrenergic receptor agonist (Schade, K. N., et al., 2015). This data is in line with literature reports that indicate a potential role for HDACs in adaptive thermogenesis and possible specific involvement of class-lla HDACs in obesity and metabolic abnormalities (Chatterjee, T. K., et al., 2014; Ferrari, A., et al., 2017; Ong, B. X., et al., 2020). However, these exciting findings warrant further studies by activating the BATs with CL316,243 to shed more light into the specific expression of class-lla HDAC isoforms in BATs and whether targeting class-lla HDACs present a therapeutic vulnerability in obesity and metabolic abnormalities. Further analysis of the metabolic profile of the new tracer is warranted to determine whether the uptake in these tissues is due to the specific uptake of the intact parent tracer or its radiometabolites.

There is also a notable intense and blockable uptake in the wrist (FIGS. 7, B and D). This uptake hints at a potential therapeutic value of class-lla HDAC inhibitors (i.e. TMP195) to treat rheumatoid arthritis and an application of the new tracer at imaging of rheumatoid arthritis considering the low background uptake in the surrounding tissues. The tracer uptake in the harderian gland is neither specific nor unique as this kind of uptake is almost universal among PET tracers. Despite many literature reports on the high expression of class-lla HDACs in the muscle, the tracer uptake was low and nonspecific (was not affected by blocking) (Ellwanger, K., et al., 2011; Fogg, P. C., et al., 2014; Hu, S., E. H. Cho, and J. Y. Lee, 2020; Liu, Y. and M. F. Schneider, 2013; Luo, L., et al., 2019; Raichur, S., et al., 2012; Simmons, B. J., et al., 2011; Usui, T., et al., 2014). The low and non-specific class lla HDAC tracer's uptake in the muscle tissues is consistent among other tracers that are currently studying as well as the data obtained with the previously reported substrate-based tracers (Bonomi, R., et al., 2015). PET is quantitative and more accurate at delineating the protein expression (density) in tissues compared to qualitative ex-vivo studies obtained by methods such as immunohistochemistry.

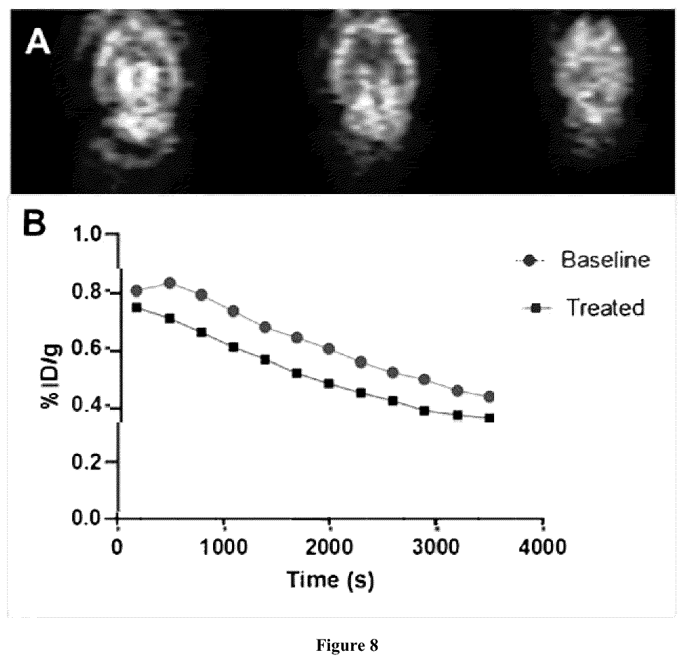

Given the specific interest in molecular imaging of class-lla HDACs in the CNS, ¹⁸F-NT57 exhibited relatively rapid uptake that peaked at ˜1% ID/g in the brain indicating a successful CNS penetration. This finding is highly significant, since it provides a proof of principle that TFMO containing molecules can penetrate the BBB and can map the class-lla HDAC expression and biodistribution in the brain. Class-lla HDACs are known to be highly expressed in various regions of the brain and their overexpression has been indicated in various brain disorders (Kikuchi, S., et al., 2015; Choi, S. Y., et al., 2018). The tracer exhibited heterogeneous biodistribution in brain regions with high uptake in the grey matter. The in vivo findings are consistent with ex-vivo findings of Broide R S, et. al. on the heterogeneous expression of class-lla HDACs in the rat brain (Trazzi, S., et al., 2016). A blocking experiment was performed to further confirm the specificity of ¹⁸F-NT57 to class-lla HDAC in vivo in the brain. The blocking study confirmed the specific binding of the tracer to class-lla HDACs in the brain. The brain uptake was reduced when comparing images obtained with the tracer administered alone (baseline) or with a cold dose of TMP195 (2 mg/kg). A time activity curve obtained over 60 min which further confirms the reduction in brain uptake due to self-blocking with TMP195 (FIG. 8, B).

### CONCLUSION

A late-stage radiosynthesis and a successful in vivo imaging with new class-lla HDAC targeting PET tracer ¹⁸F-NT57 was developed. The late-stage radiolabeling strategy produced an identical radioactive homolog of the cold class-lla HDAC inhibitor TMP195 ensuring maintenance of the identical inhibition affinity. This strategy is successfully applied to produce a new generation of ¹⁸F-TFMO containing class-lla HDAC inhibitor-based PET tracers. The single radiofluorination-step facilitate automated routine production and produces and formulates the tracer in relatively short time. The reported radiochemistry can be extended to other target molecules that contain trifluoromethyl-heterocyclic moiety. In vivo imaging with ¹⁸F-NT57 indicates a successful lead tracer for PET imaging and delineation of class-lla HDAC expression and biodistribution in rat tissues. The findings herein allow for extensive SAR studies to identify clinically translatable TEMO containing class-lla HDAC inhibitor-based PET tracer for PET imaging of cancer and the disorders of the CNS.

### REFERENCE

AI-Sanea, M. M., et al., Design, Synthesis and Biological Evaluation of New HDAC1 and HDAC2 Inhibitors Endowed with Ligustrazine as a Novel Cap Moiety. Drug Des Devel Ther, 2020. 14: p. 497-508.
Anderson K W C J, Wang M, et al. PLOS One. 2015 May 11; 10(5):e0126592.
Aramsangtienchai, P., et al., HDAC8 Catalyzes the Hydrolysis of Long Chain Fatty Acy/Lysine. ACS Chem Biol, 2016. 11(10): p. 2685-2692.
Bonomi, R., et al., Novel Histone Deacetylase Class Ila Selective Substrate Radiotracers for PET Imaging of Epigenetic Regulation in the Brain. PLOS One, 2015. 10(8): p. e0133512.
Bonomi R E L M, Popov V, Kamal S, et al. Mol Imaging Biol. 2018 August; 20(4):594-604.
Bolger T A Y T, J Neurosci. 2005 Oct. 12; 25(41):9544-53.
Burli, R. W. et al., Design, synthesis, and biological evaluation of potent and selective class lla histone deacetylase (HDAC) inhibitors as a potential therapy for Huntington's disease. J Med Chem, 2013. 56(24): p. 9934-54.
Cassetta L P J. Cell Res 2017:1-2.
Chatterjee, T. K., et al., Role of histone deacetylase 9 in regulating adipogenic differentiation and high fat diet-induced metabolic disease. Adipocyte, 2014. 3(4): p. 333-8.
Chen, Y., et al., Discovery of Novel Dual Histone Deacetylase and Mammalian Target of Rapamycin Target Inhibitors as a Promising Strategy for Cancer Therapy. J Med Chem, 2019. 62(3): p. 1577-1592.
Choi, S. Y., et al., InhibitUon of class Ila histone deacetylase activity by gal/ic acid, sulforaphane, TMP269, and panobinostat. Biomed Pharmacother, 2018. 101: p. 145-154.
Collins, L. M., et al., Class-I/a Histone Deacety/ase Inhibition Promotes the Growth of Neural Processes and Protects Them Against Neurotoxic Insult. Mol Neurobiol, 2015. 51(3): p. 1432-42.
Erburu, M., et al., Chronic stress and antidepressant induced changes in Hdac5 and Sirt2 affect synaptic plasticity. Eur Neuropsychopharmacol, 2015. 25(11): p. 2036-48.
Ellwanger, K., et al., Protein kinase D controls voluntary-running-induced skeletal muscle remodelling. Biochem J, 2011. 440(3): p. 327-4.
Fass, D. M., et al., Crebinostat: a novel cognitive enhancer that inhibits histone deacetylase activity and modulates chromatin-mediated neurop/asticity. Neuropharmacology, 2013. 64: p. 81-96.
Ferrari, A., et al., Attenuation of diet-induced obesity and induction of white fat browning with a chemical inhibitor of histone deacetylases. Int J Obes (Lond), 2017. 41 (2): p. 289-298.
Fischle W D F, Hendzel M J, et al., Mol Cell. 2002 January; 9(1):45-57.
Fitzsimons, H. L., et al., The histone deacetylase HDAC4 regulates long-term memory in Drosophila. PLOS One, 2013. 8(12): p. e83903.
Fogg, P. C., et al., Class Ila histone deacetylases are conserved regulators of circadian function. J Biol Chem, 2014. 289(49): p. 34341-8.
Gräff J T L, Nat Rev Neurosci. 2013 February; 14(2):97-111.
Griffin, E. A., Jr., et al., Prior alcohol use enhances vulnerability to compulsive cocaine self administration by promoting degradation of HDAC4 and HDACS. Sci Adv, 2017. 3(11): p. e1701682.
GTEx Portal. (2021). Gtexportal. Retrieved Feb. 14, 2022, from https://www.gtexportal.org/home/index.html
Gu, P., et al., Histone deacetylase 5 (HDAC5) regulates neuropathic pain through SRYrelated HMG-box 10 (SOX10)-dependent mechanism in mice. Pain, 2018. 159(3): p. 526-539.
Guerriero, J. L., et al., Class Ila HDAC inhibition reduces breast tumours and metastases through anti-tumour macrophages. Nature, 2017. 543(7645): p. 428-432.
Hendricks, J_A., et al., In vivo PET imaging of histone deacetylases by 1 BF-suberoylanilide hydroxamic acid (1 BF-SAHA). J Med Chem, 2011. 54(15): p. 5576-82.
Hooker, J. M., et al., Histone deacetylase inhibitor, MS-275, exhibits poor brain penetration: PK studies of [CJMS-275 using Positron Emission Tomography. ACS Chem Neurosci, 2010. 1(1): p. 65-73.
Hu, S., E. H. Cho, and J. Y. Lee, Histone Deacetylase 9: Its Role in the Pathogenesis of Diabetes and Other Chronic Diseases. Diabetes Metab J, 2020. 44(2): p. 234-244.
Kikuchi, S., et al., Class Ila HDAC inhibition enhances ER stress-mediated cell death in multiple myeloma. Leukemia, 2015. 29(9): p. 1918-27.
Kumar, J. S. D., et al., In vivo evaluation of [(11)CJTMI, a COX-2 selective PET tracer, in baboons. Bioorg Med Chem Lett, 2018. 28(23-24): p. 3592-3595.
Laws, M. T., et al., Molecular imaging HDACs class Ila expression-activity and pharmacologic inhibition in intracerebral glioma models in rats using PETICTI(MRI) with [(18)F]TFAHA. Sci Rep, 2019. 9(1): p. 3595. 36. Yeh, H. H., et al., Imaging epigenetic regulation by histone deacetylases in the brain using PET/MRI with ¹⁸F-FAHA. Neuroimage, 2013. 64: p. 630-9.
Li, L. and X. J. Yang, Molecular and Functional Characterization of Histone Deacety/ase 4 (HDAC4). Methods Mol Biol, 2016. 1436: p. 31-45.
Linares, A., et al., Increased expression of the HDAC9 gene is associated with antiestrogen resistance of breast cancers. Mol Oncol, 2019. 13(7): p. 1534-1547.
Liu, J., et al., Role of Phosphorylated HDAC4 in Stroke-Induced Angiogenesis. Biomed Res Int, 2017. 2017: p. 2957538.
Liu, W. C., et al., Environmental Stimulation Counteracts the Suppressive Effects of Maternal High-Fructose Diet on Cell Proliferation and Neuronal Differentiation in the Dentate Gyrus of Adult Female Offspring via Histone Deacetylase 4. Int J Environ Res Public Health, 2020. 17(11).
Liu, Y. and M. F. Schneider, Opposing HDAC4 nuclear fluxes due to phosphorylation by/3-adrenergic activated protein kinase A or by activity or Epac activated CaMK/1 in skeletal muscle fibres. J Physiol, 2013. 591(14): p. 3605-23.
Lobera, M., et al., Selective class Ila histone deacetylase inhibition via a nonchelating zinc-binding group. Nat Chem Biol, 2013. 9(5): p. 319-25.
Long, S., et al., Cyclo-oxygenase-1-selective inhibitor SC-560. Acta Crystallogr Sect E Struct Rep Online, 2009. 65(Pt 2): p. 0360.
Luckhurst, C. A., et al., Development and characterization of a CNS-penetrant benzhydryl hydroxamic acid class Ila histone deacetylase inhibitor. Bioorg Med Chem Lett, 2019. 29(1): p. 83-88.
Luo, L., et al., HDAC4 Controls Muscle Homeostasis through Deacetylation of Myosin Heavy Chain, PGC-1a, and Hsc70. Cell Rep, 2019. 29(3): p. 749-763.e12.
Maddox, S. A., et al., Estrogen-dependent association of HDAC4 with fear in female mice and women with PTSD. Mol Psychiatry, 2018. 23(3): p. 658-665.
Makinistoglu, M. P. and G. Karsenty, The class II histone deacetylase HDAC4 regulates cognitive, metabolic and endocrine functions through its expression in osteoblasts. Mol Metab, 2015. 4(1): p. 64-9.
Ong, B. X., et al., Regulation of Thermogenic Adipocyte Differentiation and Adaptive Thermogenesis Through Histone Acetylation. Front Endocrinol (Lausanne), 2020. 11: p. 95.
Raichur, S., et al., Histone deacetylase 5 regulates glucose uptake and insulin action in muscle cells. J Mol Endocrinol, 2012. 49(3): p. 203-11.
Rastogi, B., et al., Overexpression of HDAC9 promotes oral squamous cell carcinoma growth, regulates cell cycle progression, and inhibits apoptosis. Mol Cell Biochem, 2016. 415(1-2): p. 183-96.
Raudszus, R., et al., Fluorescent analogs of peptoid-based HOAG inhibitors: Synthesis, biological activity and cellular uptake kinetics. Bioorg Med Chem, 2019. 27(19): p. 115039.
Reid, A. E., et al., Evaluation of 6-([(18)F]fluoroacetamido)-1-hexanoicanilide for PET imaging of histone deacetylase in the baboon brain. Nucl Med Biol, 2009. 36(3): p. 247-58.
Saha, P., et al., Histone Deacetylase 4 Downregulation Elicits Post-Traumatic Psychiatric Disorders through Impairment of Neurogenesis. J Neurotrauma, 2019. 36(23): p. 3284-3296.
Sando R G N, Pieraut S, et al. Cell. 2012 Nov. 9; 151(4):821-834.
Schade, K. N., et al., Preliminary evaluation of {33-adrenoceptor agonist-induced 18F-FDG metabolic activity of brown adipose tissue in obese Zucker rat. Nucl Med Biol, 2015. 42(8): p. 691-4.
Schwartz, S., et al., Long-Term Memory in Drosophila Is Influenced by Histone Deacetylase HDAC4 Interacting with SUMO-Conjugating Enzyme Ubc9. Genetics, 2016. 203(3): p. 1249-64.
Seo, Y. J., et al., Radionuclide labeling and evaluation of candidate radioligands for PET imaging of histone deacetylase in the brain. Bioorg Med Chem Lett, 2013. 23(24): p. 6700-5.
Simmons, B. J., et al., HDACs in skeletal muscle remodeling and neuromuscular disease. Handb Exp Pharmacol, 2011. 206: p. 79-101.
The Human Protein Atlas. (2021 November 18). The Human Protein Atlas. Retrieved Feb. 14, 2022, from https://www.proteinatlas.org./
Trazzi, S., et al., HDAC4: a key factor underlying brain developmental alterations in CDKLS disorder. Hum Mol Genet, 2016. 25(18): p. 3887-3907.
Turkman N, S. A., Paolillo V, et al. Nucl Med Biol. 2012 May; 39(4):593-600.
Turkman, N., et al., Synthesis and preliminary evaluation of [1 BFJ-labeled 2-oxoquinoline derivatives for PET imaging of cannabinoid CB2 receptor. Nucl Med Biol, 2012. 39(4): p. 593-600.
Uchida, S. and G. P. Shumyatsky, Synaptically Localized Transcriptional Regulators in Memory Formation. Neuroscience, 2018. 370: p. 4-13.
Usui, T., et al., Histone deacetylase 4 controls neointimal hyperplasia via stimulating proliferation and migration of vascular smooth muscle cells. Hypertension, 2014. 63(2): p. 397-403.
Van de Bittner, G. C., E. L. Ricq, and J. M. Hooker, A philosophy for CNS radiotracer design. Ace Chem Res, 2014. 47(10): p. 3127-34.
Wanek, J., et al., Pharmacological Inhibition of Class/IA HOA Cs by LMK-235 in Pancreatic Neuroendocrine Tumor Cells. Int J Mol Sci, 2018. 19(10).
Wang, C., et al., In vivo imaging of histone deacetylases (HDACs) in the central nervous system and major peripheral organs. J Med Chem, 2014. 57(19): p. 7999-8009.
Wang, Z., et al., A small molecular compound CC1007 induces cross-lineage differentiation by inhibiting HDAC7 expression and HDAC7/MEF2C interaction in BCRABL1(−) pre-B-ALL. Cell Death Dis, 2020. 11(9): p. 738.
Wey H Y, G. T., Zurcher N R, et al. Sci Transl Med. 2016 Aug. 10; 8(351):351ra106.
Wey, H. Y., et al., Kinetic Analysis and Quantification of {11C]Marchtinostat for in Vivo HDAC Imaging of the Brain. ACS Chem Neurosci, 2015. 6(5): p. 708-15.
Yeh H H Y D, Gelovani J G, et al. Brain Res. 2013 Apr. 4; 1504:16-24.
Younes, A., et al., Safety, tolerability, and preliminary activity of CUDC-907, a first-in-class, oral, dual inhibitor of HDAC and P/3K, in patients with relapsed or refractory lymphoma or multiple myeloma: an open-label, dose-escalation, phase 1 trial. Lancet Oncol, 2016. 17(5): p. 622-31.
Yu, D., et. al., VEGF-PKD1-HDAC7 signaling promotes endothelial progenitor cell migration and tube formation. Microvasc Res, 2014. 91: p. 66-72.
Yuan, Y., et al., Screening of novel histone deacetylase 7 inhibitors through molecular docking followed by a combination of molecular dynamics simulations and ligand-based approach. J Biomol Struct Dyn, 2019. 37(15): p. 4092-4103.
Zeglis, B. M., et al., The synthesis and evaluation of N1-(4-(2-[18FJ-fluoroethyl)phenyl)-N8-histone deacetylase expression in cancer. Nucl Med Biol, 2011. 38(5): p. 683-96.
Zhang, S., et al., Selenophenes: Introducing a New Element into the Core of Non-Steroidal Estrogen Receptor Ligands. ChemMedChem, 2017. 12(3): p. 235-249.

1. A compound having the structure: 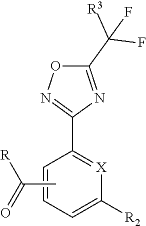
wherein:
X is C or N;
R is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkyIOC₁₋₆alkyl, halogen, or —C(O)OtButyl;
R² is H or halogen; and
R³ is halogen;
wherein when R² is F, R³ is F, X is C, and R and R² are in ortho position, R is other than 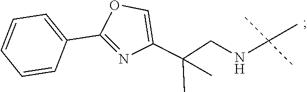
wherein when R² is H, R³ is F, X is N, and R and R² are in ortho position, R is other than 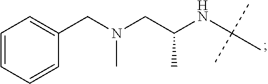
wherein when R² is F, R³ is F, X is C, and R and R² are in ortho position, R is other than 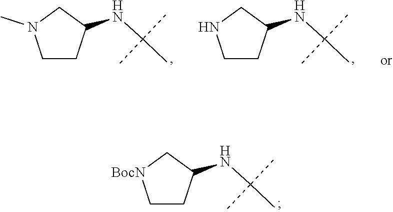
or
wherein when R² is H, R³ is F, X is C, and R and R² are in ortho position, R is other than OH or

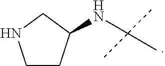

2. A composition comprising a mixture of compound A having the structure: 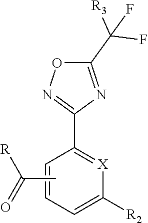
and
compound B having the structure: 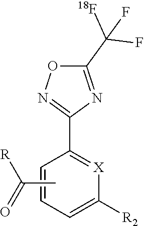
wherein:
X is C or N;
R is H, OH, —O—C₁₋₆ alkyl, formyl, carbonyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen;
wherein when R² is F, R³ is F, X is C, and R and R² are in ortho position, R is other than 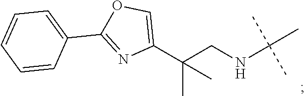
wherein when R² is H, R³ is F, X is N, and R and R² are in ortho position, R is other than 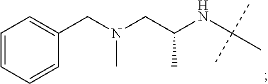
wherein when R² is F, R³ is F, X is C, and R and R² are in ortho position, R is other than 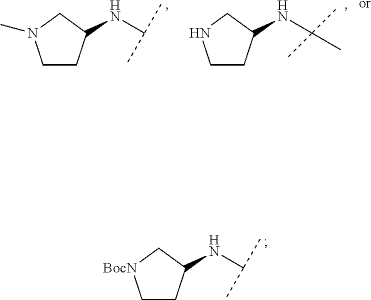
or
wherein when R² is H, R³ is F, X is C, and R and R² are in ortho position, R is other than OH or

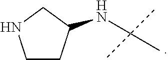

3. A process for preparing a compound III having the structure: 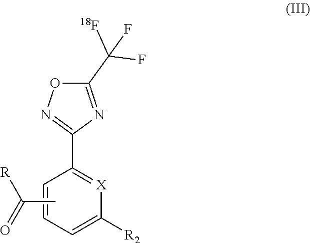
comprising
a) reacting R—H with compound IIIa having the structure: 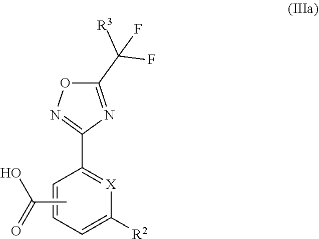
in 1-[Bis(dimethylamino)methylene]-1H-1,2,3-triazolo[4,5-b]pyridinium 3-oxid hexafluorophosphate (HATU), 4-methylmorpholine (NMM) and dimethylformamide (DMF) to obtain compound IIIb having the structure: 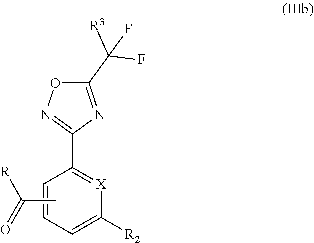
b) reacting compound IIIb with a [¹⁸F] fluorinating agent in a solvent to obtain compound III having structure: 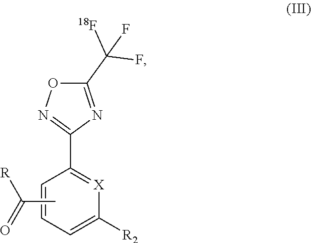
wherein:
X is C or N;
R is H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl;
R² is H or halogen; and
R³ is halogen.

4. A process for preparing compound IV(a) having the structure: 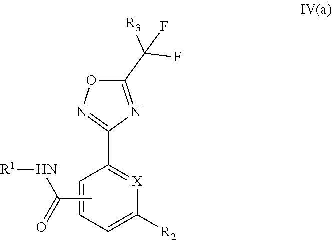
comprising:
a) reacting compound I(a) having the structure: 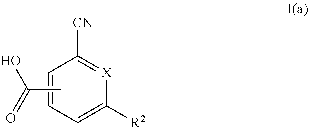
with hydroxylamine with water and ethanol to obtain compound II(a) having the structure: 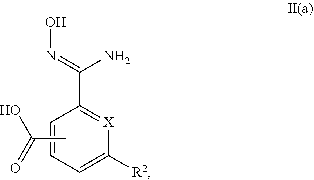
b) reacting compound II(a) with 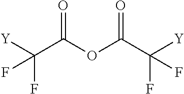
in pyridine to obtain compound IIIc having the structure: 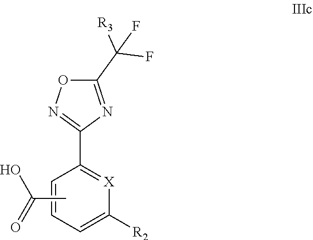
c) reacting compound IIIc with an amine in 1-[Bis(dimethylamino)methylene]-1H-1,2,3-triazolo[4,5-b]pyridinium 3-oxid hexafluorophosphate (HATU) and an excess amount of 4-methylmorpholine (NMM) to produce compound IVa having the structure: 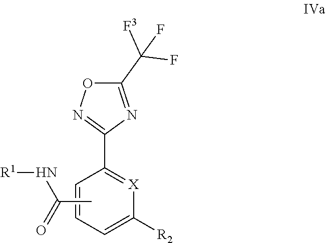
wherein:
X is C or N;
R¹ is selected from the group consisting of H, OH, O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkyIOC₁₋₆alkyl, halogen, or —C(O)OtButyl;
R² is H or halogen;
R² is halogen; and
Y is halogen.

5. A process for preparing compound IV having the structure: 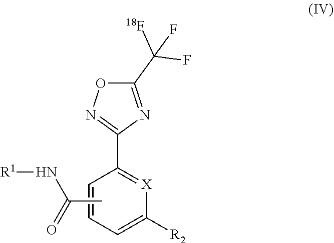
comprising:
a) reacting compound I having the structure: 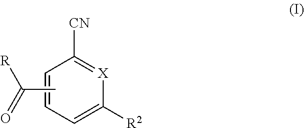
with hydroxylamine to obtain compound II having the structure: 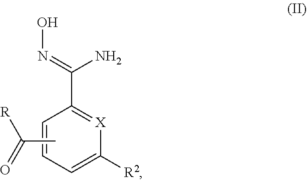
b) reacting compound II with bromodifluoroacetic anhydride in pyridine to obtain compound IIIb having the structure: 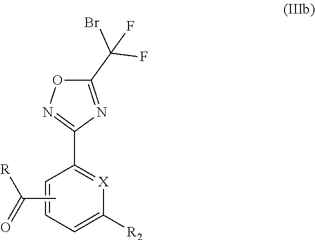
c) reacting compound IIIb with an amine in 1-[Bis(dimethylamino)methylene]-1H-1,2,3-triazolo[4,5-b]pyridinium 3-oxid hexafluorophosphate (HATU) and an excess amount of 4-methylmorpholine (NMM) to produce compound IVb having the structure: 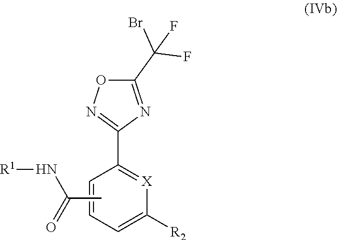
d) further reacting compound IVb with a [¹⁸F] fluorinating agent in a solvent to obtain compound IV having the structure: 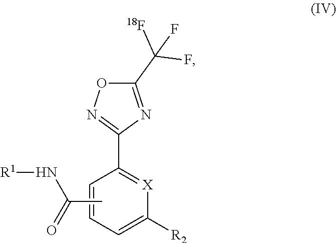
wherein:
X is C or N;
R is H, OH, O—C₁₋₆ alkyl, carbonyl, formyl or R¹;
R¹ is selected from the group consisting of —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl; and
R² is H or halogen.

6. The process of claim 5, wherein compound IV has the structure:

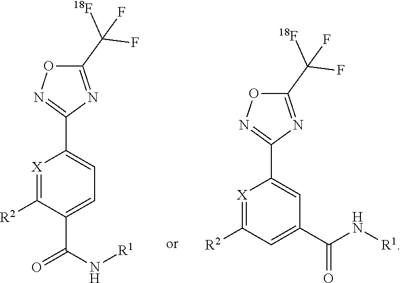

7. The process of claim 5, wherein compound IIIb is reacted with Cs ¹⁸F and K₂₂₂ in DMSO to obtain compound II having the structure: 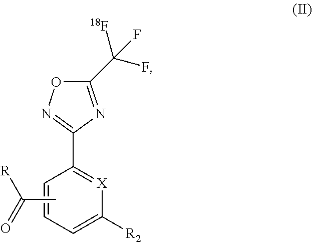
then compound II is further reacted with NaOH to produce compound V having the structure:

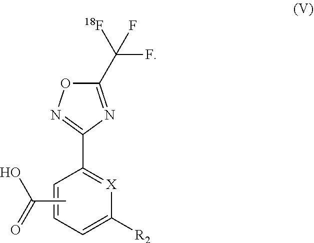

8. The process of claim 5, wherein compound IIIb is 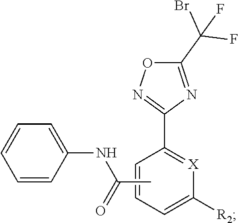
wherein compound IVb is 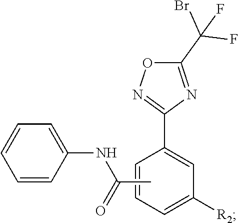
or wherein compound IV is

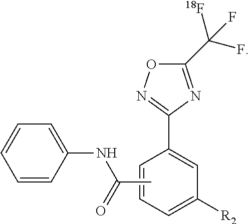

9. The process of claim 7, wherein compound Vis

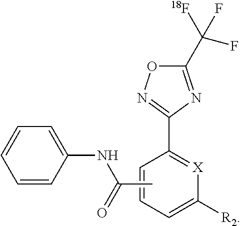

10. The process of claim 5, wherein amine in step (c) is benzyl amine.

11. The process of claim 3 or 5, wherein the [¹⁸F] fluorinating agent in a solvent is Cs ¹⁸F and K₂₂₂ in N, N-dimethylacetamide (DMA) or dimethyl sulfoxide (DMSO) or ¹⁸F-fluoride (K¹⁸F) in dimethyl sulfoxide (DMSO).

12. The process of claim 11, wherein DMSO is added at a temperature range from 145° C. to 160° C.

13. The process of claim 12, wherein DMSO is added at 150° C.

14. The composition of claim 2, wherein the composition contains about 1-10% compound B by weight.

15. The composition of claim 14, wherein the composition contains 2-5% compound B by weight.

16. A method of detecting a disease of the central nervous system (CNS) in a subject, the method comprising:
a) administering compound in claim 1 in a mammal;
b) detecting the presence of compound in the mammal using positron emission tomography (PET).

17. The compound of claim 1, the composition of claim 2, or the process of claim 3 or 5, wherein R and R² or R and R¹ are in ortho position.

18. The compound of claim 1, the composition of claim 2, or the process of claim 3 or 5, wherein R and R² or R and R¹ are in meta position.

19. The compound of claim 1, the composition of claim 2, or the process of claim 3 or 5, wherein R and R² or R and R¹ are in para position.

20. The compound of claim 1, the composition of claim 2, or the process of claim 3 or 5, wherein R³ is F, Cl, Br or I.

21. The compound of claim 1, the composition of claim 2, or the process of claim 3 or 5, wherein R³ is radioactive.

22. The compound of claim 1, the compound B in claim 2, or the compound III in claim 3 having the structure: 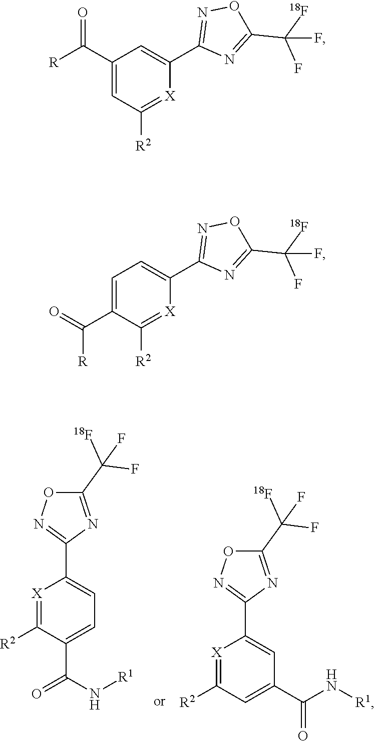
wherein R or R¹ is OH, —O—C₁₋₆ alkyl, carbonyl, formyl, —C₁₋₆alkyl, —C₁₋₆alkenyl, —C₁₋₆alkynyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, —C₀₋₆alkylNHC₀₋₆alkyl heterocyloalkyl, C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-N-heteroaryl-heteroaryl, C₁₋₆alkyl-N-aryl-heteroaryl, C₁₋₆alkyl-N-aryl-aryl, C₁₋₆alkyl-heteroaryl-aryl, C₁₋₆alkyl-heteroaryl-heteroaryl, C₁₋₆alkyl-aryl-aryl, C₁₋₆alkyl-aryl-heteroaryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl;
each of which is optionally substituted by one or more substituents selected from —C₀₋₆alkyl, —C₁₋₆alkylOC₁₋₆alkyl, halogen, or —C(O)OtButyl.

23. The compound of claims 17-22, wherein R or R¹ is independently H, OH, methyl, formyl, carbonyl or —O—C₁₋₆ alkyl.

24. The compound of claims 17-22, wherein R or R¹ is C₁₋₆alkylC(O)NC₁₋₆alkyl aryl, C₁₋₆alkyl-N—C₁₋₆alkyl aryl, C₁₋₆alkyl-N-heteroaryl-aryl, C₁₋₆alkyl-heteroaryl-aryl, C₁₋₆alkylC(O)—C₁₋₆alkyl-NH—C₁₋₆alkyl aryl, C₁₋₆alkylC(O)—NH—C₁₋₆alkyl aryl, cyclic amine, cyclic amine aryl, cyclic amine-C₁₋₆alkyl-aryl, C₁₋₆alkyl-cyclic amine, C₁₋₆alkyl-cyclic amine aryl, C₁₋₆alkylC(O)-cyclic amine or C₁₋₆alkylC(O)-cyclic amine aryl.

25. The compound of claim 24, wherein R or R¹ is C₁₋₆alkylC(O)NC₁₋₆alkyl phenyl, C₁₋₆alkyl-N—C₁₋₆alkyl phenyl, C₁₋₆alkyl-N-oxazole-phenyl, C₁₋₆alkyl-oxazole-phenyl, C₁₋₆alkylC(O)—C₁₋₆alkyl-NH—C₁₋₆alkyl phenyl, pyrrolidine, pyrrolidine phenyl, C₁₋₆alkyl-pyrrolidine, C₁₋₆alkyl-pyrrolidine-phenyl, C₁₋₆alkylC(O)-pyrrolidine, C₁₋₆alkylC(O)-pyrrolidine-phenyl, piperidine, piperidine phenyl, C₁₋₆alkyl-piperidine, piperidine-C₁₋₆alkyl-phenyl, C₁₋₆alkylC(O)-piperidine, C₁₋₆alkylC(O)-piperidine-phenyl.

26. The compound of claim 25, wherein the oxazole, phenyl, pyrrolidine and piperidine is further substituted with halogen, C₁₋₆alkyl, aryl, or C₁₋₆alkyl aryl.

27. The compound of claims 17-22, wherein R or R¹ is —O—C₁₋₆ alkyl.

28. The compound of claim 27, wherein R or R¹ is —O-methyl, —O-ethyl or —O-tert-butyl.

29. The compound of claims 17-28, wherein R² is F, Cl, Br or I.

30. The compound of claims 17-28, wherein R² is H.

31. The compound of claims 17-28, wherein R² is F.

32. The compound of claims 17-31, wherein X is C.

33. The compound of claims 17-31, wherein X is N.

34. The compound of claims 17-22, wherein R or R¹ contains a radioactive label.

35. The compound of claim 34, wherein the radioactive label is a halogen or carbon.

36. The compound of claims 17-22, wherein R or R¹ is selected form the group consisting of:

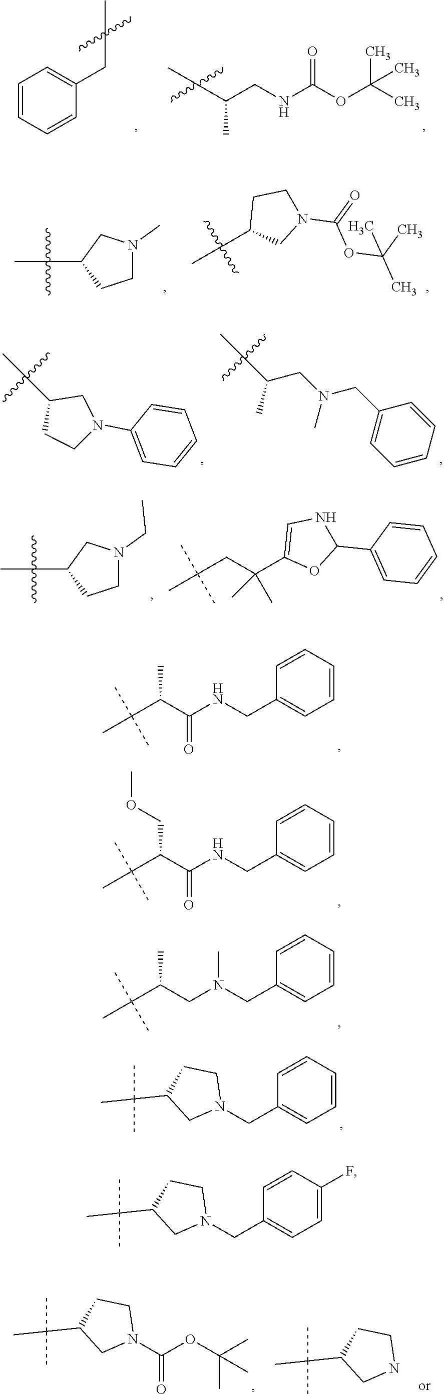

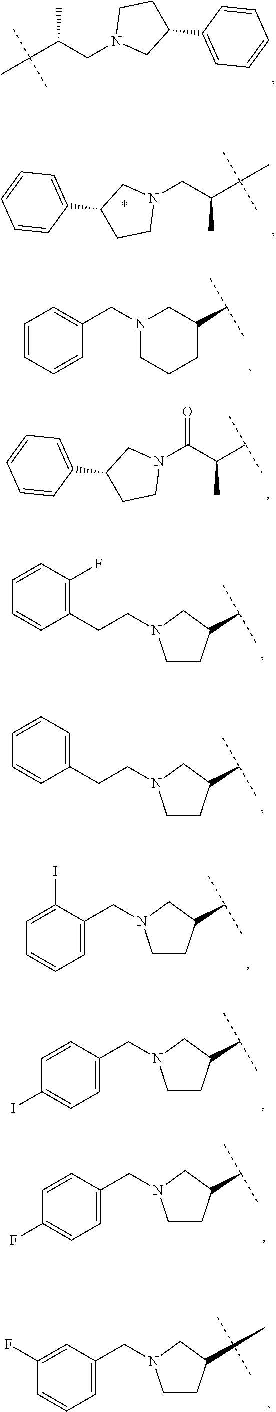

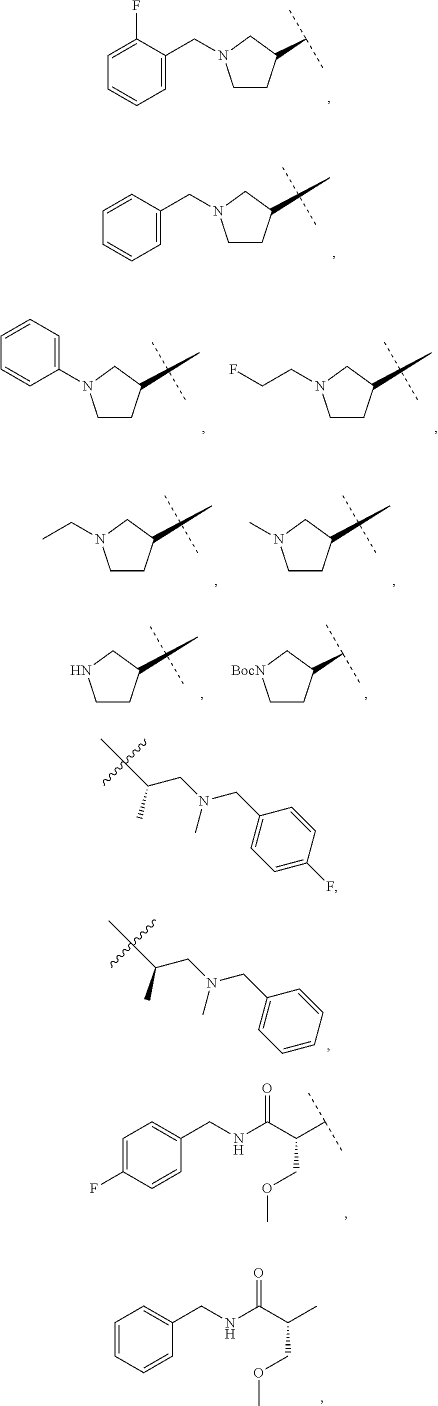

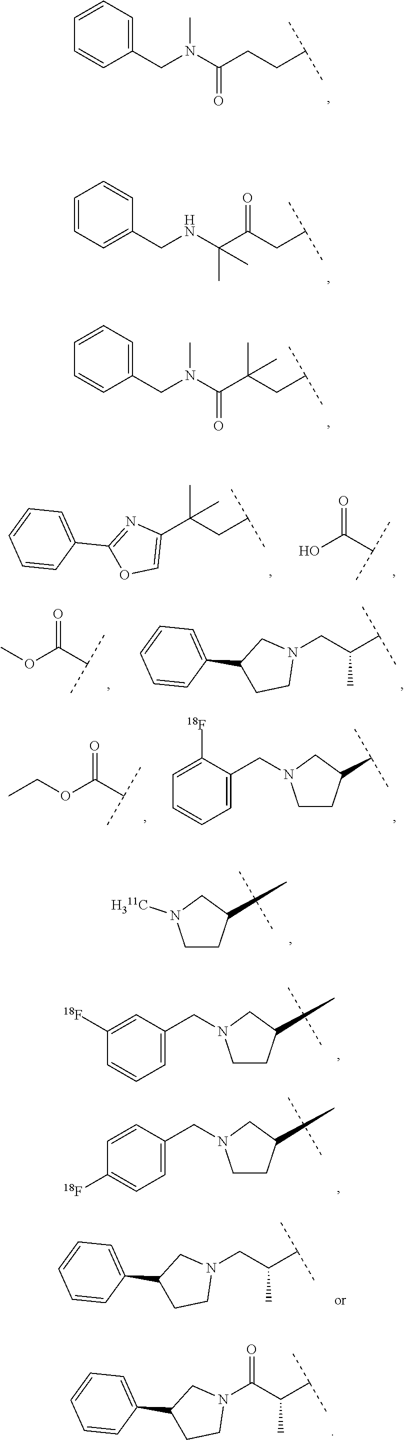

37. The compound of claim 1 having the structure:

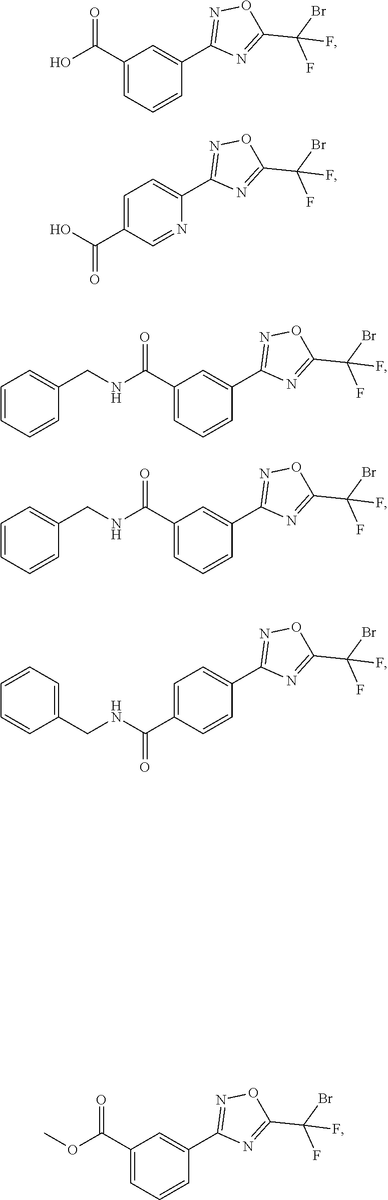 [SMILES: O=C(O)c1cccc(-c2noc(C(F)(F)Br)n2)c1 ; O=C(O)c1ccc(-c2noc(C(F)(F)Br)n2)nc1 ; O=C(NCc1ccccc1)c1cccc(-c2noc(C(F)(F)Br)n2)c1 ; O=C(NCc1ccccc1)c1cccc(-c2noc(C(F)(F)Br)n2)c1 ; O=C(NCc1ccccc1)c1ccc(-c2noc(C(F)(F)Br)n2)cc1 ; COC(=O)c1cccc(-c2noc(C(F)(F)Br)n2)c1]

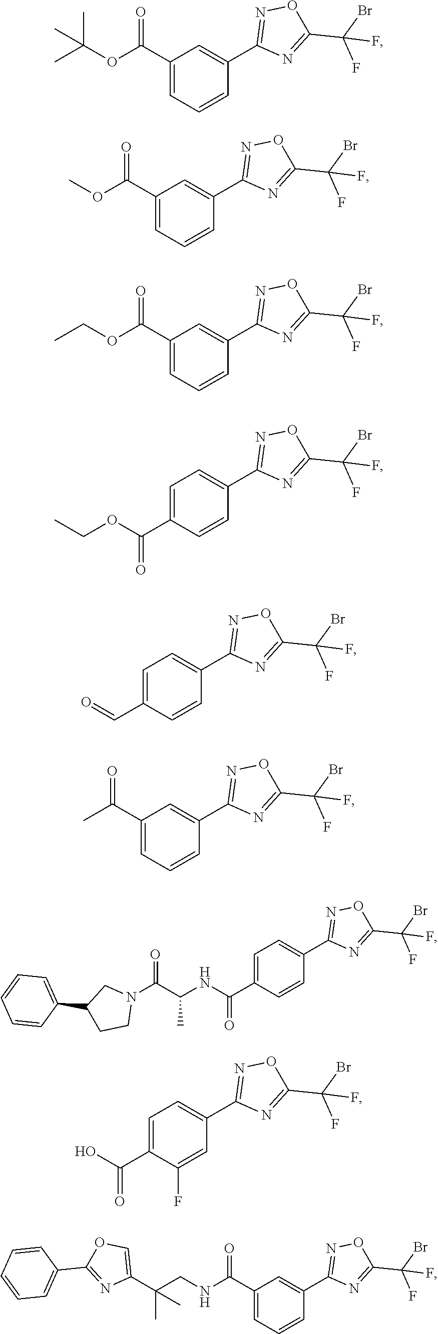

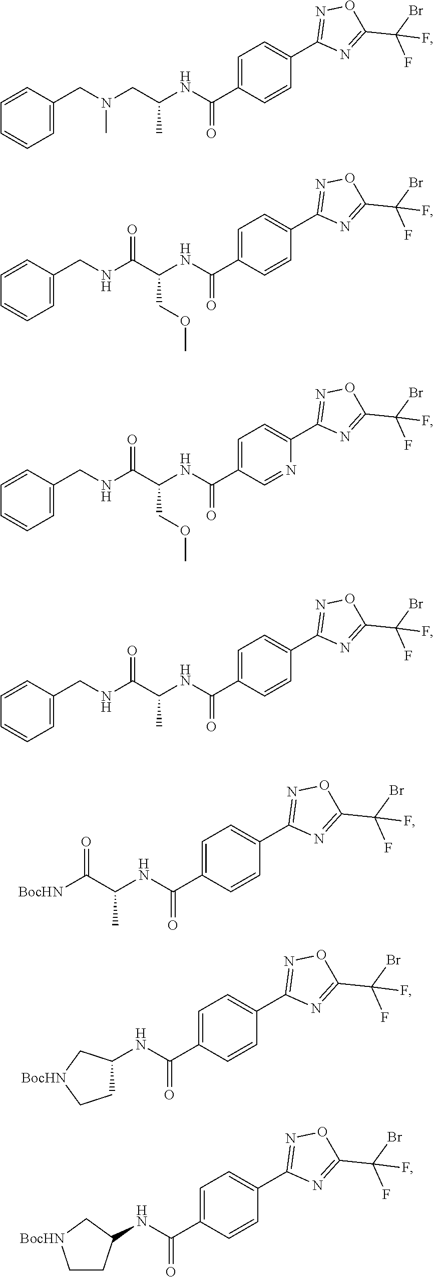

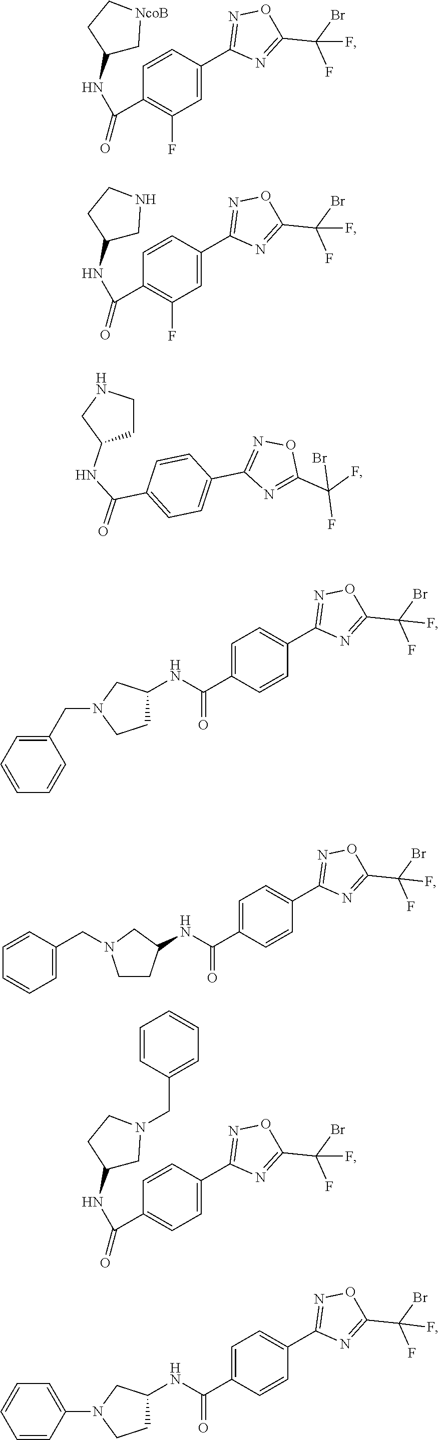

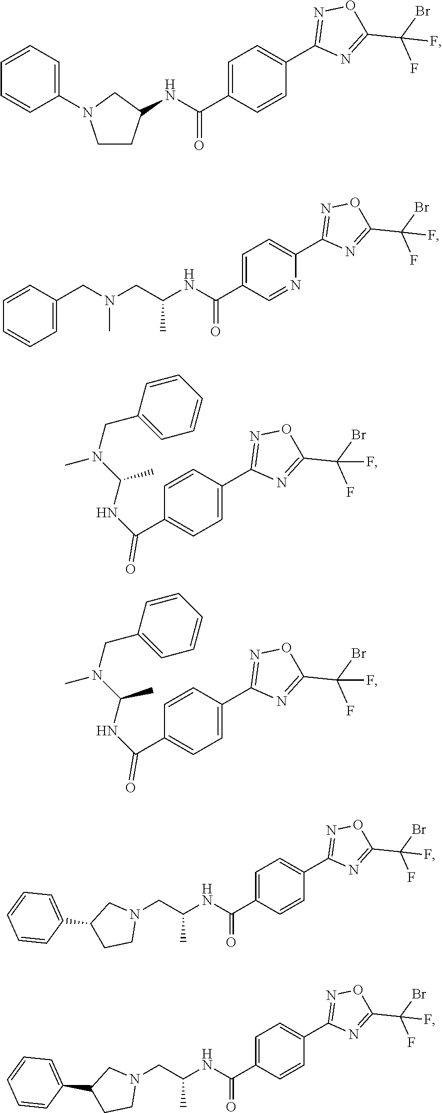

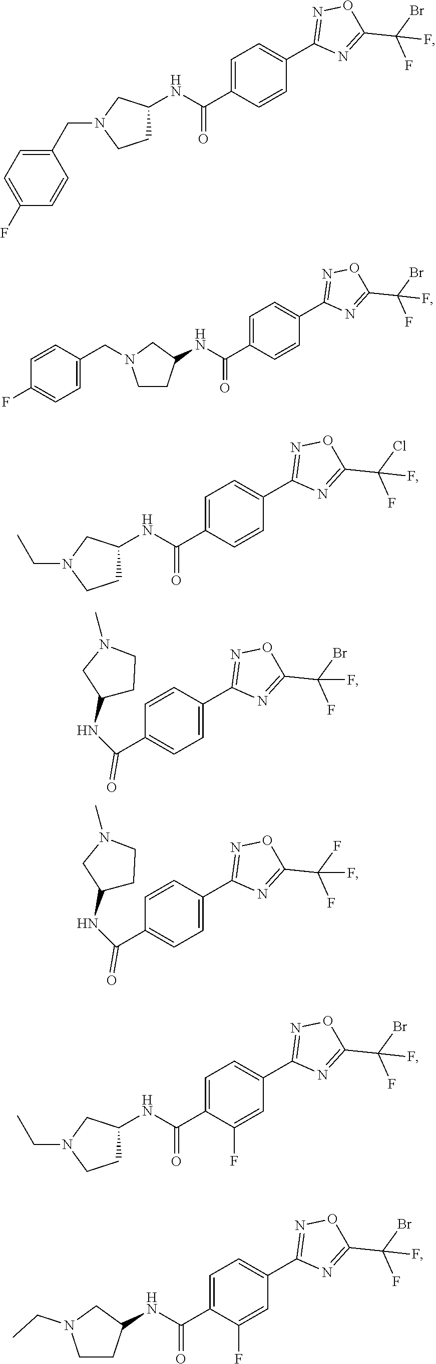

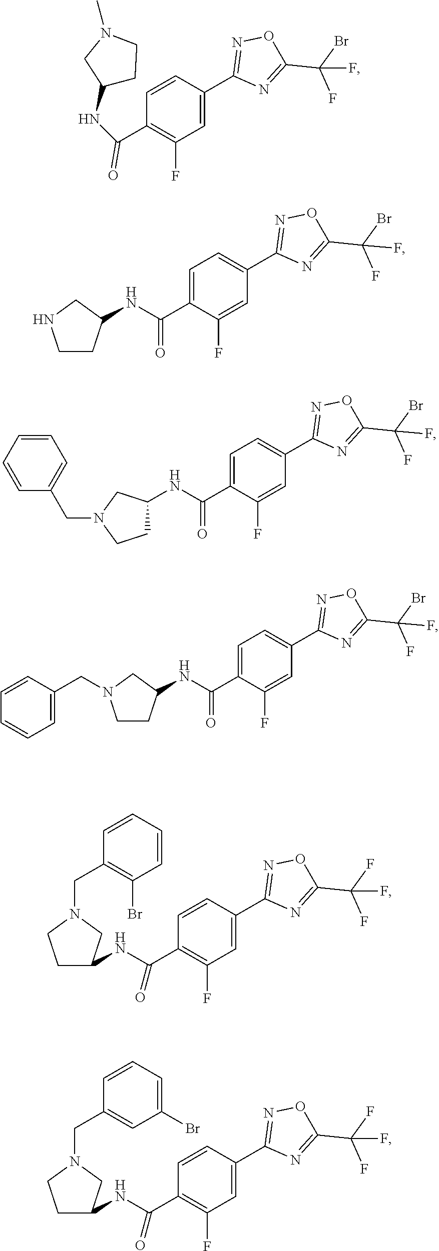

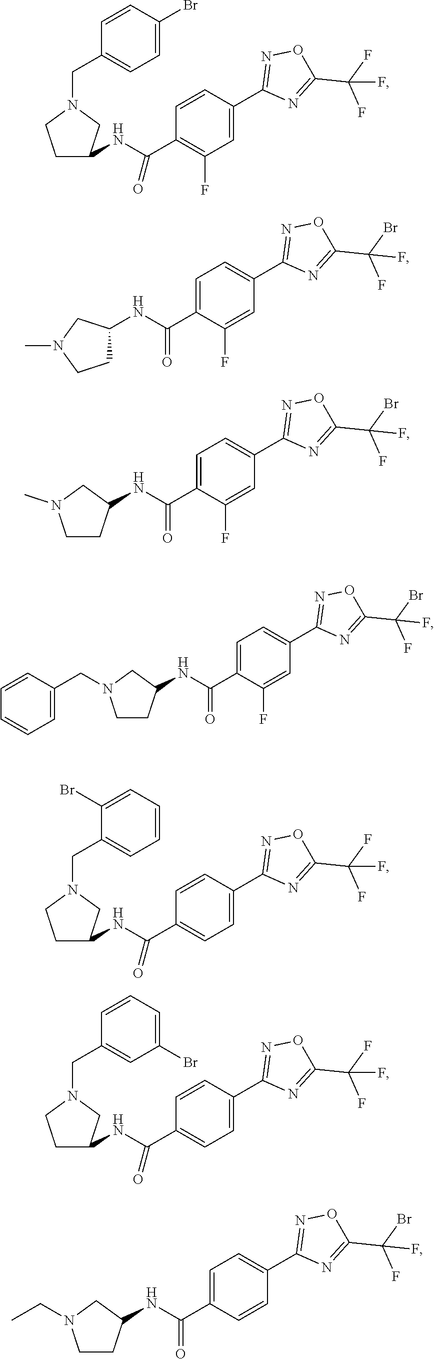

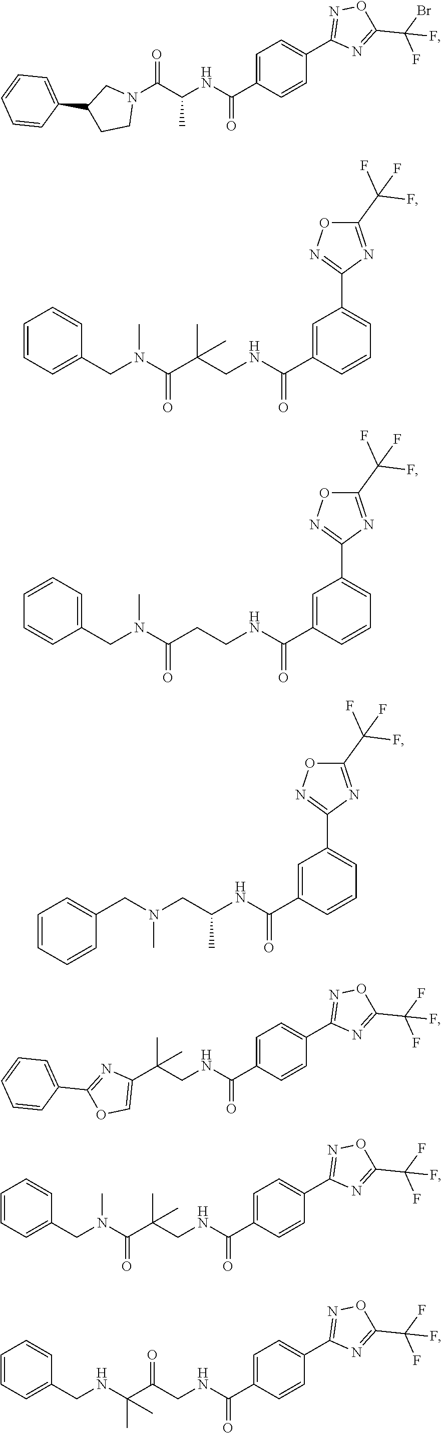

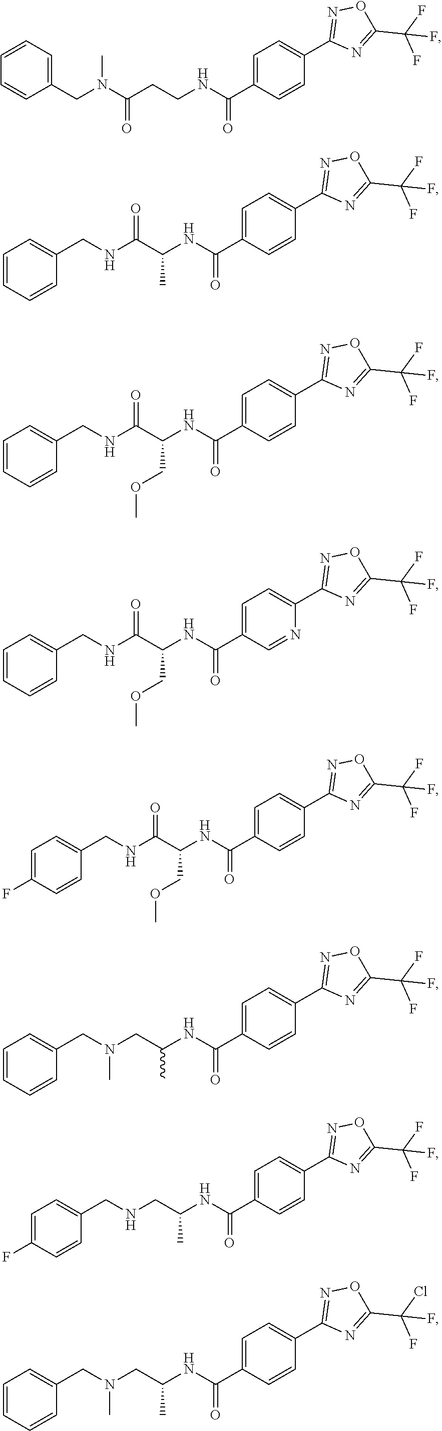

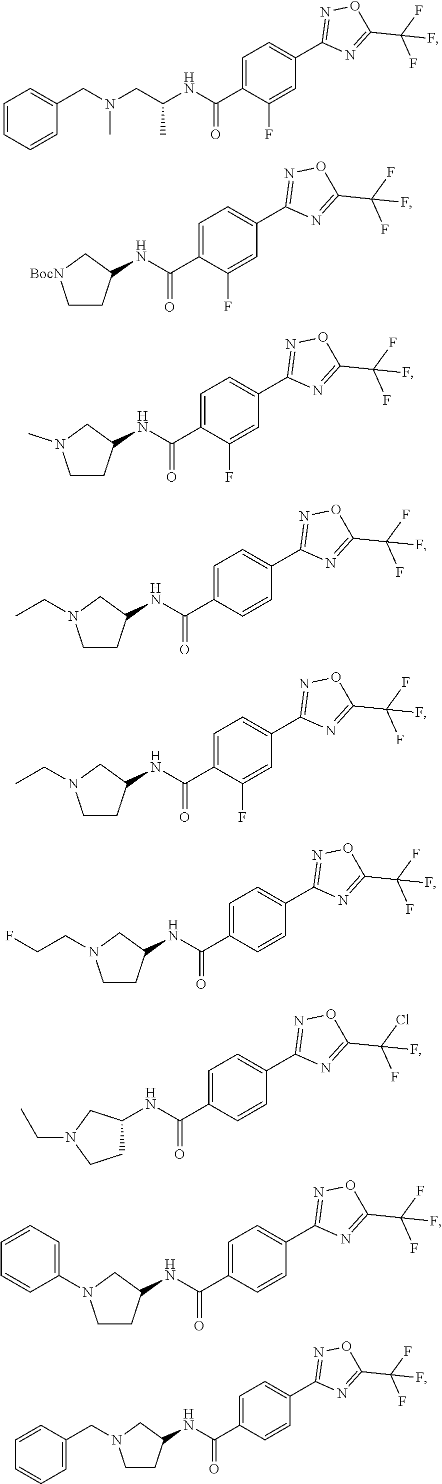

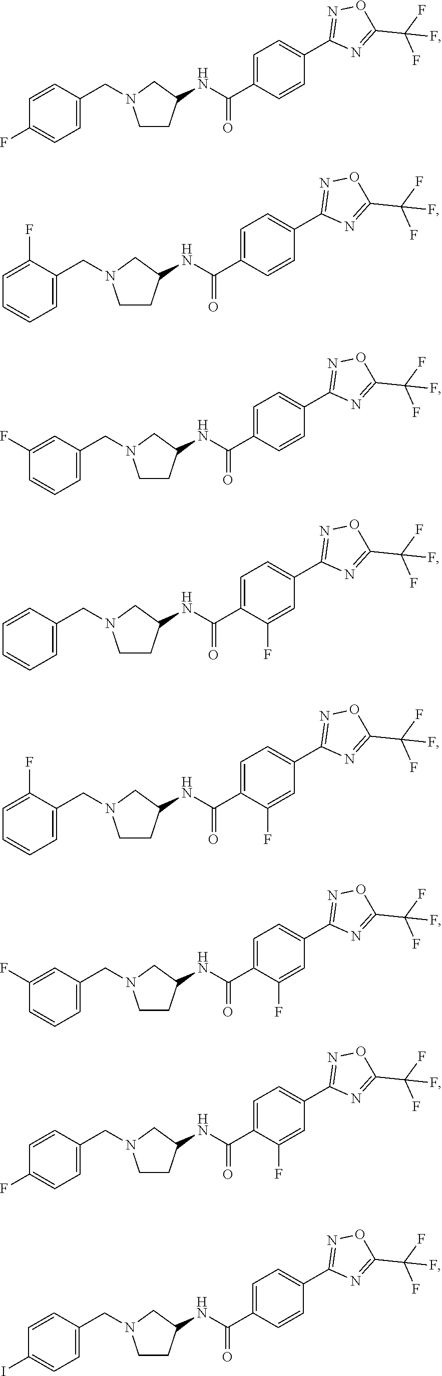

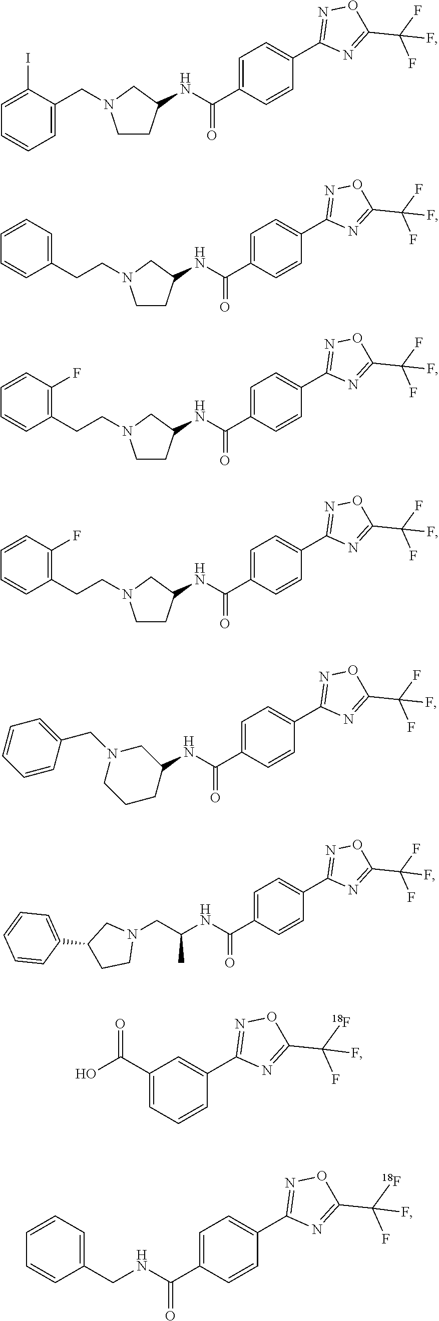

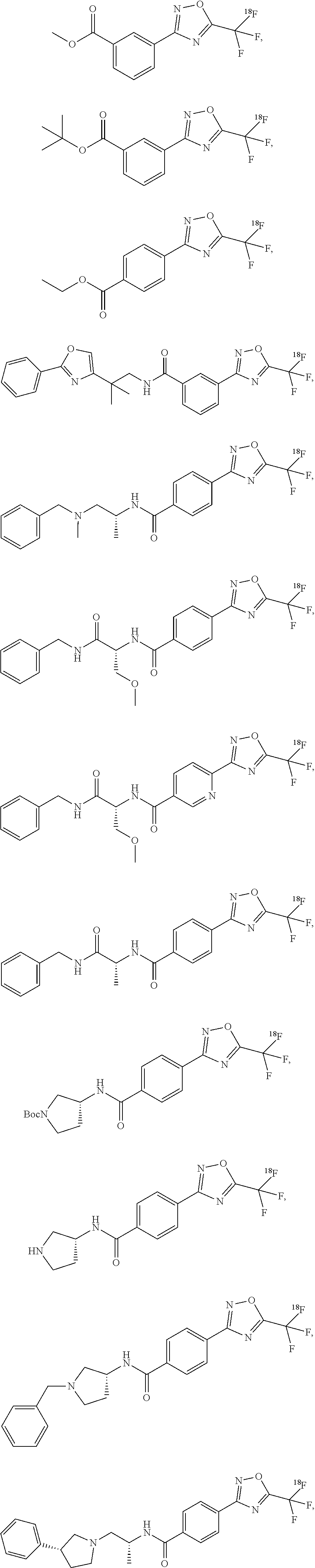

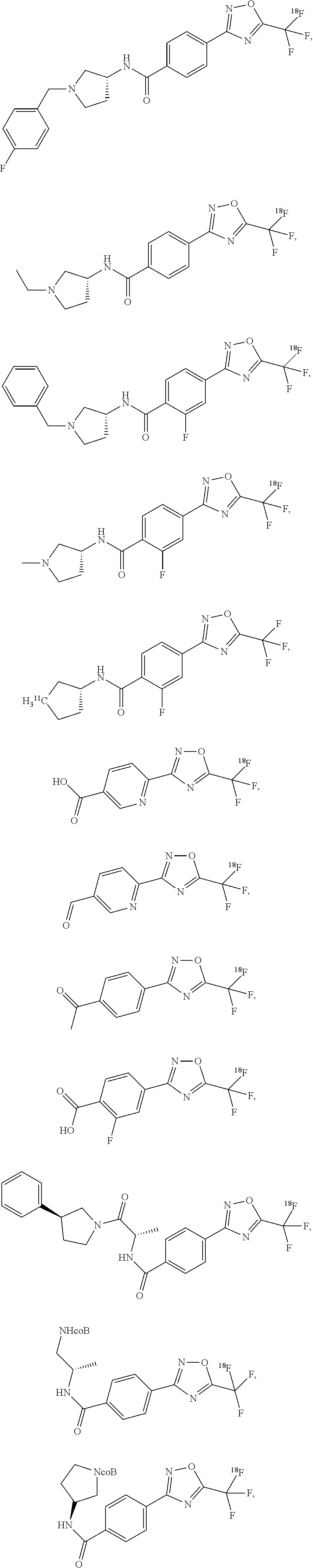

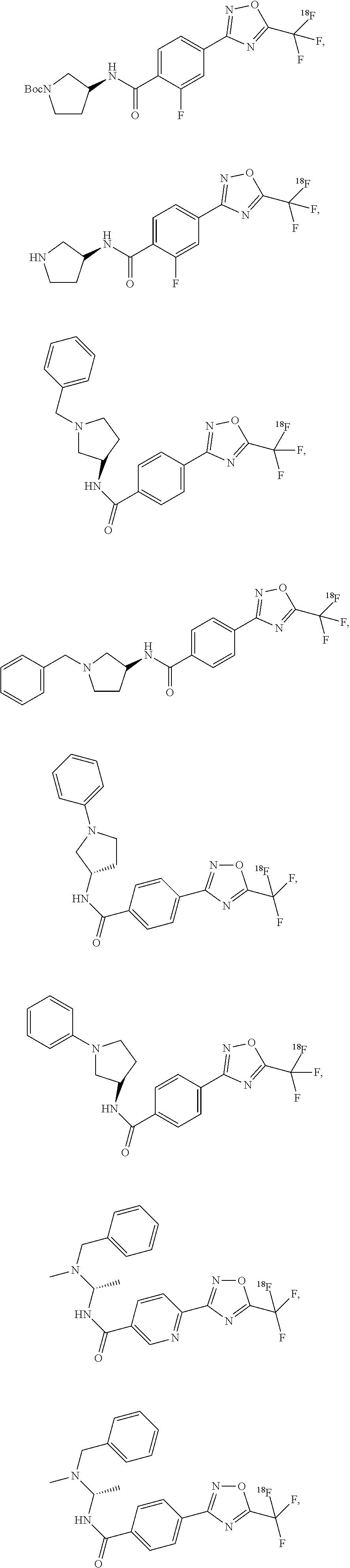

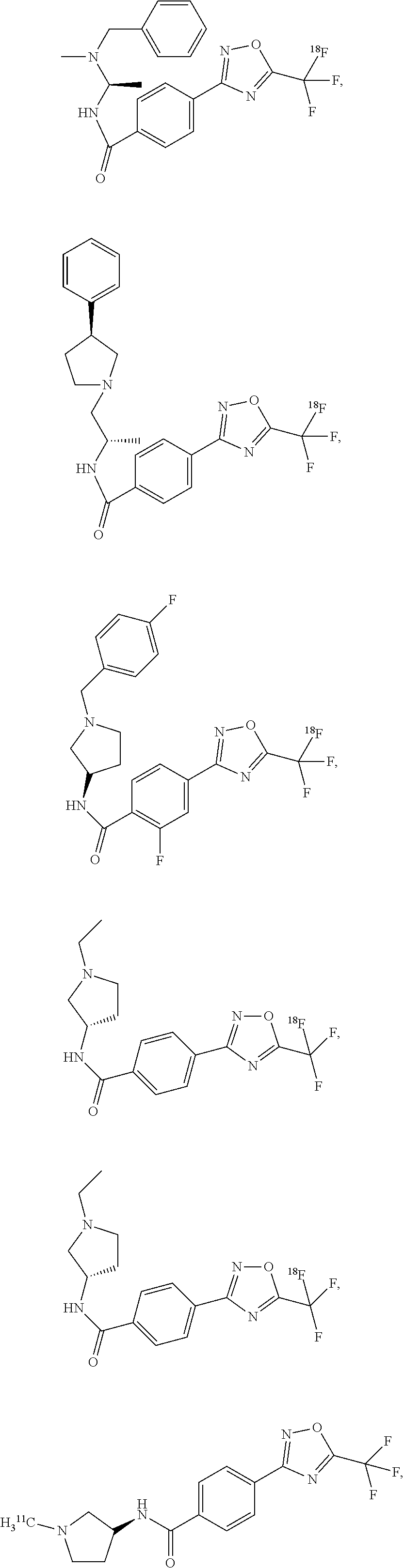

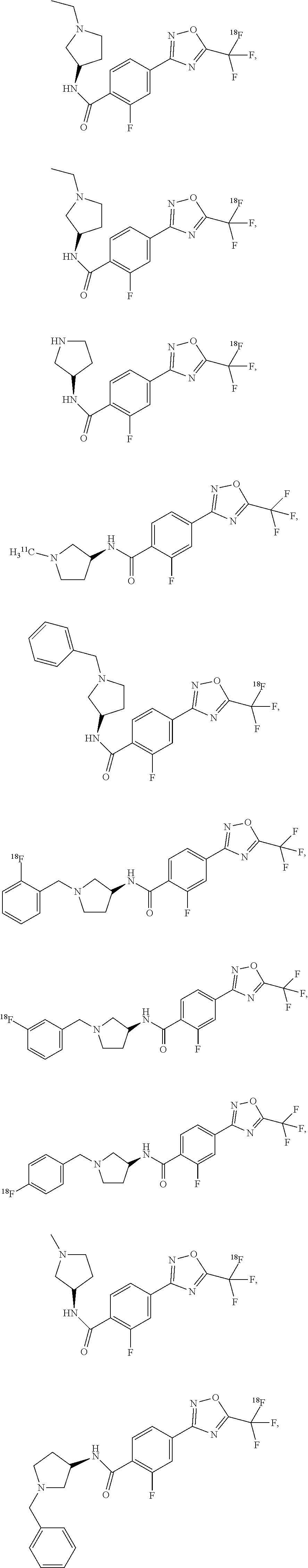

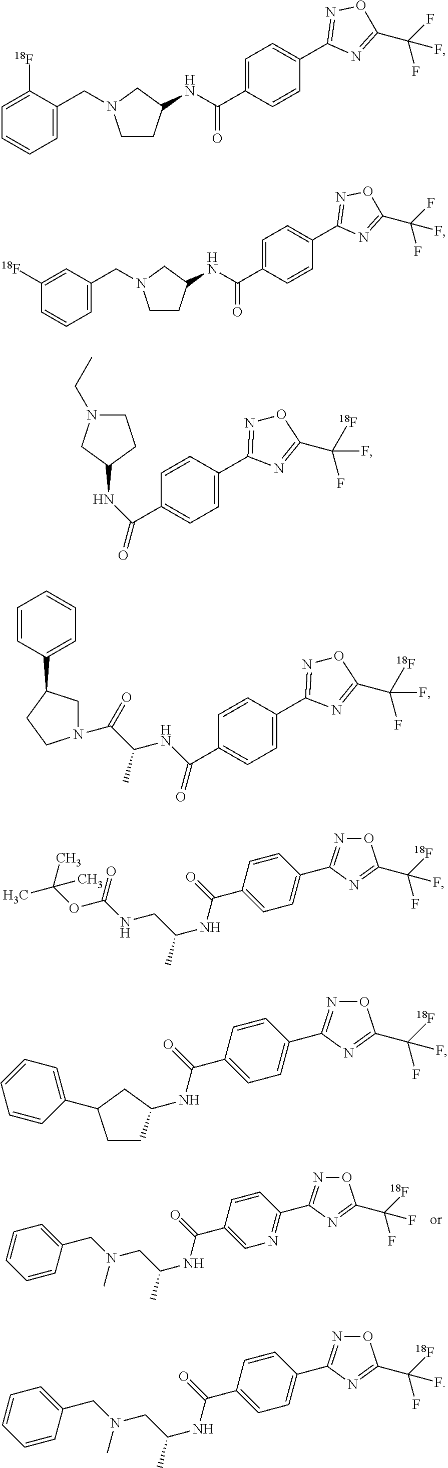

38. The compound of claims 17-37, wherein the compounds are human histone deacetylases (HDACs) inhibitors.

39. The compound of claim 38, wherein HDACs are HDAC-4, HDAC-5, HDAC-7 and HDAC-9.

40. The process of claim 4, wherein R² and Y is independently F, Cl, Br or I.

41. The method of claim 16, wherein the mammal is a rodent.

42. The method of claim 41, wherein the rodent is a rat.

43. The method of claim 16, wherein the compound is a HDAC inhibitor and wherein the HDAC inhibitors has blood-brain barrier (BBB) permeability.

44. The method of claim 43, wherein the HDAC inhibitor is further used for brain imaging.

45. The method of claim 16, wherein the disease is cancer, disorders of the central nervous system, memory and cognitive impairment, dementia, rheumatoid arthritis or behavioral changes.

46. The method of claim 43, wherein the HDAC inhibitor accumulated in the liver of a mammal.

47. The method of claim 43, wherein the HDAC inhibitors accumulated in the brown fat adipose tissues (BATs).

48. A method of identifying a HDAC inhibitor which comprises creating a library of candidate HDAC inhibitors containing a ¹⁸F radiolabeled group, using the candidate HDAC inhibitors to PET image an organ of a subject, obtaining the biodistribution of the HDAC inhibitors in the organ of the subject, and identifying the HDAC inhibitor based on the biodistribution of the organ of the subject.

49. A composition of the formula: 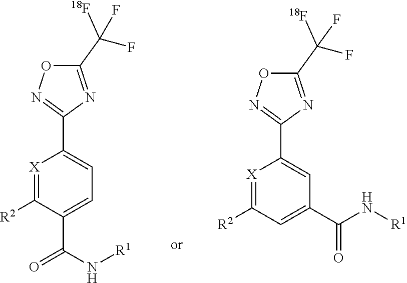
wherein:
X is selected from CH and N;
R¹ is selected from —C₁₋₆alkyl, —C₀₋₆alkyl aryl, —C₀₋₆alkyl heteroaryl, —C₀₋₆alkyl cycloalkyl, —C₀₋₆alkyl heterocyloalkyl, —C₀₋₆alkylC(O)NHC₀₋₆alkyl, —C₀₋₆alkylC(O)NHC₀₋₆alkyl aryl, —C₀₋₆alkylC(O)NHC₀₋₆alkyl heteroaryl, —C₀₋₆alkylC(O)NHC₀₋₆alkyl cycloalkyl, —C₀₋₆alkylC(O)NHC₀₋₆alkyl heterocyloalkyl, —C₀₋₆alkylNHC₀₋₆alkyl, —C₀₋₆alkylNHC₀₋₆alkyl aryl, —C₀₋₆alkylNHC₀₋₆alkyl heteroaryl, —C₀₋₆alkylNHC₀₋₆alkyl cycloalkyl, and —C₀₋₆alkylNHC₀₋₆alkyl heterocyloalkyl, each of which is optionally substituted by one or more substituents selected from —C₀₋₆alkyl, —C₁₋₆alkylOC₁₋₆alkyl, halogen, and —C(O)OtButyl; and
R² is H or halogen.

50. The composition according to claim 1, wherein R is selected from:

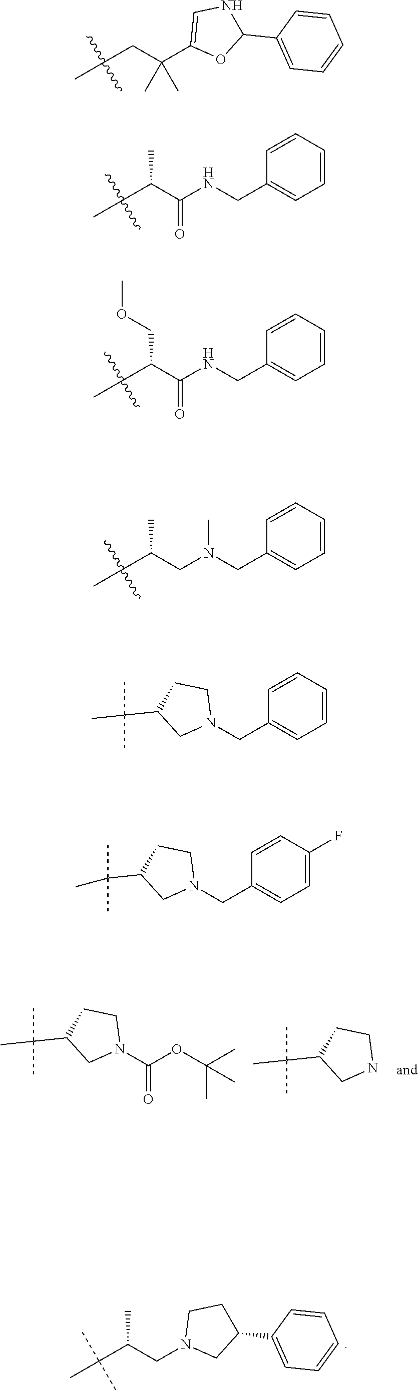

51. A method of detecting a disease of the central nervous system in a subject, the method comprising:
administering a radiolabeled histone deacetylase (HDAC) inhibitor according to claim 49 or 50 to the subject;
detecting the presence of the radiolabeled HDAC inhibitor in the subject using positron emission tomography (PET).

52. A method of synthesizing a composition, the method comprising:
adding Cs¹⁸F and K₂₂₂ to a solution of a composition of the formula: 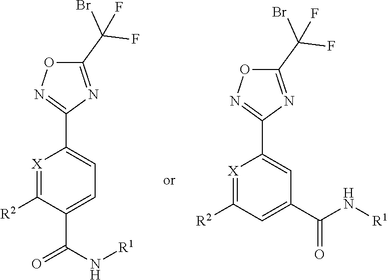
heating the solution to about 100-200° C. to afford the composition according to claim 49 or 50.
